(19) United States
(12) Patent Application Publication
(10) Pub. No.: US 2020/0018763 A1
(43) Pub. Date: Jan. 16, 2020

(54) METHODS TO ASSESS BINDING AGENT SPECIFICITY

(71) Applicants: OSLO UNIVERSITETSSYKEHUS HF, Oslo (NO); UNIVERSITETET I OSLO, Oslo (NO)

(72) Inventors: Fridtjof LUND-JOHANSEN, Eiksmarka (NO); Krzsysztof SIKORSKI, Oslo (NO); Marit INNGJERDINGEN, Oslo (NO); Adi MEHTA, Oslo (NO)

(73) Assignees: Oslo Universitetssykehus HF, Oslo (NO); Universitetet i Oslo, Oslo (NO)

(21) Appl. No.: 16/335,774

(22) PCT Filed: Sep. 26, 2017
(86) PCT No.: PCT/EP2017/074416

§ 371 Date: Mar. 22, 2019

(30) Foreign Application Priority Data
Sep. 26, 2016 (GB) 1616313.1

(51) Int. Cl. G01N 33/58; G01N 33/68; G01N 33/53; C07D 495/04; C07J 19/00

(52) U.S. Cl. CPC G01N 33/58; G01N 33/6848; C07J 19/00; C07D 495/04; G01N 33/53

(57) ABSTRACT

The present invention relates to methods for assessing binding agent specificity, in particular antibody specificity. The present invention thus provides a method of analysing a mixture of polypeptides comprising the steps of: (i) separating the polypeptides in the mixture into a plurality of fractions; (ii) contacting a first aliquot of two or more of the fractions with a plurality of different binding agents attached to one or more solid supports and detecting the binding of the polypeptides to the binding agents in each fraction; (iii) assessing the amino acid composition of the polypeptides in a second aliquot of said fractions by mass spectrometry; and (iv) correlating the binding results detected in step (ii) and the mass spectrometry results from step (iii) to assess the specificity of the binding agents for a polypeptide of interest.

### FIELD OF THE INVENTION

This invention relates to a method of analysing a mixture of polypeptides and for example assessing the specificity or sensitivity of one or more binding agents for a polypeptide of interest within the mixture of polypeptides.

### BACKGROUND

Polypeptide (or protein) binding agents such as antibodies are used in a wide range of applications to detect polypeptides (or proteins) in research and diagnostics. The majority of antibodies used today are made by immunisation of animals with a polypeptide or a polypeptide fragment. Monoclonal antibodies are made by immortalisation of immune cells and are therefore renewable. Polyclonal antibodies are isolated from animal serum. These reagents are non-renewable, and since the outcome of immunisation with a given target is unpredictable, each production lot is in reality a different reagent.

Ideally, a binding agent (or affinity reagent) binds strongly and specifically to the target it was raised against. However, the performance of polypeptide binders is unpredictable (Marx, V, Nat. Methods, 10, 14 (2013)). Off-target binding is common, and researchers often find that antibodies they purchase yield little or no signal. A widely cited study on 5000 commercially available antibodies (Berglund, L. et al., Mol. Cell. Proteomics, 7, 2019-2027 (2008)) showed that less than half were useful in commonly used applications such as western blotting (WB) and immunohistochemistry (IHC).

Researchers who seek an antibody to a given polypeptide can perform searches in web-based portals such as Antibodypedia.org, Biocompare.com and CiteAb.com and retrieve a list of alternative products from a large number of vendors. However, it is difficult to assess the relative performance of the reagents from the information provided in the product specification sheets (Marx, V, supra). The sheets typically contain images of results obtained in applications such as WB, Immunofluorescence microscopy (IF) and IHC. There is no industry standard for testing, and images are poorly suited for comparison of parameters such as signal strength. In many cases, antibodies with different quality may seem to perform similarly.

It is well known that antibody performance varies with applications and samples (Marx, V, supra). Ideally, manufacturers should therefore test their entire product line in a wide range of applications and samples. However, extensive testing is expensive, and since most antibodies generate little revenue, it is not cost-effective to perform rigorous validation. Researchers must therefore often base their choice of product on validation data from an application or sample different from the one they intend to use the product in. A large and widely cited study concluded that it was “impossible” to predict the performance of an antibody in one application from results obtained in another (Marx, V, supra; and Algenas, C. et al., Biotechnol. J., 9, 435-445 (2014)). The implication is that researchers often purchase one reagent after the other until they find one that suits their needs (Bradbury, A & Pluckthun A, Nature, 518, 27-29 (2015); Marx, V, supra; and Baker, M., Nature, 527, 545-551 (2015)).

Since customers cannot predict the performance of antibodies from the information in manufacturers' product specification sheets, they rely on the only objective parameter available, which is the number of times an antibody has been cited in the scientific literature. Citation statistics for more than a million commercially available antibodies are now freely accessible in web-based search engines such as Citeab.com. A Citeab search for antibodies to a popular target such as the epidermal growth factor receptor (EGFR) shows that there are a few antibodies with a very large number of citations and a very large number that have none. The top-cited product (sc03) is a polyclonal antibody that has been on the market for decades. As explained above, polyclonal antibodies are non-renewable and each production lot is in reality a different reagent. In this case, the manufacturer keeps the same catalogue number for a series of production lots that are likely to be very different, and it is highly unlikely that all these lots are consistently superior to all competitor products. Thus, the lack of robust and transparent criteria for antibody performance prevents free competition by providing early-appearing products with an unfair advantage in the market.

The funds wasted on the purchase of poor quality antibodies have been estimated to be $700 M in the United States alone (Bradbury, A & Pluckthun A, supra; Marx, V, supra; and Baker, M., supra). Poorly validated antibodies are also expected to yield a large number of irreproducible results, and the costs of irreproducible laboratory research have been estimated to be $28 Bn. This problem is now receiving considerable attention in media and research organizations. For example, the Human Proteome Organization (HUPO) has appointed a committee of experts to provide guidelines for standardized antibody validation, and their recommendations are expected to be published in 2016. Improvements in and standardisation of antibody validation is important to industry, academia, scientific journals and government agencies including the NIH.

It is generally recommended that antibodies are validated in the application they are to be used in (Bradbury, A & Pluckthun A, supra; Marx, V, supra; and Baker, M., supra). Product specification sheets in the catalogues of leading antibody vendors such as Atlas® antibodies (www.proteinatlas.org), Abcam® (www.abcam.com), Thermo Fisher Scientific® (https://www.thermofisher.com/no/en/home/life-science/antibodies/primary-antibodies.html), Sigma Aldrich® (http://www.sigmaaldrich.com/life-science/cell-biology/antibodies.html) and Cell Signalling Technologies (www.cellsignal.com) therefore typically contain images obtained after use in applications such as WB, IF and IHC.

In an attempt to standardise the evaluation of such images, the web portal Antibodypedia.org has established criteria for assessing results obtained in each application. These criteria are based on those used for the Human Protein Atlas (HPA), which is the largest project world-wide to produce and validate antibodies against human polypeptides (www.proteinatlas.org). The Antibodypedia guidelines were used as basis for guidelines published by an International Working Group for Antibody Validation (IWGAV) in 2016 (Uhlen et al. Nat Methods. 13:823-827 (2016).

Western Blotting (WB):

Antibody manufacturers commonly use WB as a first test of specificity. The procedure is straightforward: sample polypeptides are denatured, separated according to size by gel electrophoresis, transferred to a membrane and labeled with an antibody. Binding of antibodies to sample polypeptide is observed as bands on the membrane, and the position of the band corresponds to the intended antibody target is predictable from its DNA sequence (i.e. predicted mass). Extra bands are often observed, and these may indicate that the antibody cross-reacts with other polypeptides. The Antibodypedia has recommendations for assessment of specificity in WB, but due to inherent limitations of the assay (see the section below relating to shortcomings of current practice), there is a large room for subjective interpretation of the results, and there are no guidelines for assessment of sensitivity.

Immunohistochemistry (IHC) and Immunofluorescence Microscopy (IF):

The assays report on the distribution of the antibody target in tissues, cells and subcellular compartments. Staining patterns in IHC can to a certain extent be predicted from available data on mRNA levels in tissues, while published information about the subcellular distribution can be used to predict staining patterns in IF (Antibodypedia.org). However, while several large studies on the distribution of polypeptides in human organs have been published, there have not been attempts to compare the results to determine if the results are similar (Kim, M. S. et al., Nature, 509, 575-581 (2014); Uhlen, M. et al., Science, 347, 1260419 (2015) and Wilhelm, M. et al., Nature, 509, 582-587 (2014)). Also, there is very little consensus regarding the localisation of polypeptides in subcellular compartments. Staining patterns in IF and IHC are therefore not nearly as predictable as that those in WB. Many antibody manufacturers therefore use WB as a generic specificity test, and antibodies that appear specific in WB are selected for use in IHC and IF.

There are shortcomings in relation to the current practice methods, as described below.

### Western Blotting (WB):

The error margin for mass estimation in WB is in the order of 20% (antibodypedia.org), and a large number of polypeptides have similar mass (www.Uniprot.org). A band at, for example, 40 kDa can therefore represent thousands of different polypeptides. Ideally, the blot used to validate an antibody shows results obtained with comparable samples that are known to contain the intended target or not. However, in most cases it is not feasible to find bona fide positive- and negative controls among commonly studied cell types since only a few polypeptides have well-established cell type-restricted expression.

An international working group for antibody validation (IWGAV) has recommended the use of targeted gene disruption to obtain bona fide negative controls (Uhlen et al 2016, supra). Alternatively, one may prepare a WB using proteins from a series of different cell types and measure differential expression of the antibody target as variation in the intensity of the bands. Proteins from the same cell types may be analysed by mass spectrometry to obtain a reference for differential protein expression. If the antibody recognizes its intended target, one expects to observe a correlation between band intensity and MS data for the intended antibody target. Currently, there is very little data published to demonstrate the utility of this approach. The use of WB as a general method to validate antibodies is also limited by the fact that many reagents useful for IHC and IF bind to conformation-dependent epitopes that are lost during sample processing for WB.

Immunohistochemistry (IHC) and Immunofluorescence Microscopy (IF):

As explained above, there is no definitive and comprehensive source of information about the distribution of polypeptides in subcellular compartments. The largest study on gene transcription in human organs show that only 200 polypeptides are exclusive for one tissue and 95% of these are in the testis. It is therefore difficult to predict staining patterns that correspond to specific binding in IF and IHC.

Certain measures have been taken in order to overcome the shortcomings of the commonly used assays.

Product specification sheets for antibodies typically show images where antibodies have been used one at a time. This type of testing is laborious and expensive. Attempts have therefore been made to enhance throughput through development of multiplexed assays where large numbers of antibodies are used in parallel.

Multiplexed Western Blotting:

In standard WB, antibodies are used one at a time. Jones and co-workers describe a miniaturised multiplexed version where a single large gel is organised into 96 individual microgels, each with six lanes for sample polypeptides (US 20110028339 A1). The approach allows parallel testing of up to 96 antibodies for binding of polypeptides of up to six different cell types. Templin and co-workers describe a high throughput version of WB where the blot is divided physically into small fragments, and the immobilized polypeptides are eluted into liquid fractions (US 20140248715 A1). The polypeptides are next immobilized to latex microspheres with addressable bar codes. A given bar code corresponds to polypeptides with a specified narrow range of physical characteristics such as size. A plurality of differently coded microspheres is contacted with a single soluble antibody specificity. After staining with a fluorescent reporter molecule, the microspheres are analyzed by flow cytometry. Since flow cytometric analysis of fluorescence has a wide dynamic range and the results have a numerical format, the method should provide more precise information about antibody sensitivity than what can be obtained from a WB image.

Multiplexed Immunoprecipitation:

Lund-Johansen and co-workers describe a method for multiplexed immunoprecipitation of biotinylated polypeptides that have been separated according to physical parameters or subcellular location (WO 2009080370). Published applications include a combination of subcellular fractionation and size exclusion chromatography (SEC). This method is often referred to as SEC-MAP (SEC-resolved Microsphere Affinity Proteomics) Holm, A., Wu, W. & Lund-Johansen, F., New biotechnology, 29, 578-585 (2012)). In MAP, antibodies are coupled to polymer microspheres with addressable fluorescent bar codes (WO 2007008084). Biotinylated sample polypeptides that have been captured onto the surface of the microspheres are labeled with fluorescent streptavidin directly on the bead surface, and the microspheres are analysed using a flow cytometer capable of reading the fluorescent bar codes and measuring streptavidin fluorescence from captured polypeptides. The SEC-MAP approach yields size distribution profiles for the targets of thousands of antibodies in parallel. Specific binding is detected as the overlap in the reactivity profiles obtained with two or more different antibodies to the same polypeptide.

Methods have also been developed in order to better determine the specificity of a binding agent.

Targeted Gene Disruption (Knockout, KO, Knockdown KD):

Certain antibody manufacturers including UK-based Abcam have implemented targeted gene disruption in their validation pipeline, and the webportal Antibodypedia has launched an initiative to encourage researchers to do the same. Samples where the target gene has been successfully disrupted represent the current gold standard for negative controls. In principle, such samples can be used in any assay.

Dual Epitope Validation:

Two antibodies that bind to different parts (epitopes) of the same polypeptide rarely cross-react with the same polypeptides. Assays where a signal is obtained only when both antibodies bind simultaneously to the same polypeptides are therefore highly specific. Variations of this validation are listed below:
a) ELISA: A well-known example is Enzyme Linked Immunosorbent Assays (ELISA) where an antibody bound to a solid phase (capture antibody) is used to capture its target from solution. A second antibody binding to a different epitope is used for detection.
b) Proximity Ligation Assays (PLA): In PLA, antibodies are coupled to nucleotides. If the two bind in proximity of each other, the nucleotides can be ligated using an enzyme to generate a continuous strand. Enzymatic approaches for DNA amplification are next used to selectively amplify the continuous strand.
c) Immunoprecipitation and Western Blotting (IP-WB): An antibody coupled to a bead support (e.g. agarose, or polymer beads) is used to capture its target from solution. The captured polypeptide is released and detected by WB using an antibody that binds to a different epitope of the same polypeptide. In this assay, two different antibodies to the same polypeptide are used in serial rather than simultaneous binding.

Immunoprecipitation and Mass Spectrometry (IP-MS):

An antibody coupled to a bead support (such as agarose, or polymer beads) is used to capture its target from solution. The captured polypeptide(s) are released and detected by Liquid-chromatography Mass Spectrometry (LC-MS/MS). LC-MS/MS yields sequence-based identification of captured polypeptides. A recent and thorough study published in the prestigious journal Nature Methods showed that IP-MS is useful to provide definitive evidence that antibodies bind to their intended targets (Marcon, E. et al., Nat Methods, 12, 725-731 (2015)).

There are shortcomings in relation to this technology, as described below.

Multiplexed Western Blotting:

While multiplexed versions of the WB enhance the throughput, the limitations with regard to assessment of specificity are the same as in standard WB. The assays resolve antibody binding against polypeptide size, but since many polypeptides have similar size, this does not constitute definitive validation.

Multiplexed Immunoprecipitation:

The SEC-MAP method allows parallel use of large numbers of antibodies, and there is evidence that reactivity profiles of different antibodies to the same polypeptide overlap to the extent that they cluster as nearest neighbors in hierarchical cluster analysis. However, this reference is only valid if the antibodies detect different epitopes, and in most cases, antibody epitopes are uncharacterized. Definitive validation by SEC-MAP therefore requires access to samples that can be used as positive and negative controls.

Targeted Gene Disruption:

Targeted gene disruption cannot be applied on primary human cells and tissue samples. Techniques for targeted gene disruption such as RNA interference and CRISPR are also very expensive and laborious. This is likely to be a reason why the number of reagents that have been tested on cells or tissues with targeted gene disruption is very small. It also seems unlikely that knockdown techniques will be part of standard validation in the foreseeable future. Finally, targeted gene disruption is not an assay, but a method used to obtain negative control samples. Results obtained in any assay are simpler to interpret, but the challenges associated with assessment of sensitivity are not affected by knock-down approaches.

Dual Epitope Validation:

It is often difficult to find two antibodies capable of binding simultaneously to different parts of the same polypeptide (matched antibody pairs). Most likely, this is the reason why dual epitope validation is rarely performed in the industry.

Immunoprecipitation and Mass Spectrometry (IP-MS):

This technique was recently promoted as the new “gold standard” for antibody validation. However, IP-MS has very low throughput. Typically, a single LC-MS/MS run occupies a highly expensive instrument for three to four hours. Interpretation of IP-MS data is also very complex. The end result is typically a list of 200 or more polypeptides, and only a small fraction of these correspond to antibody targets. The reason is that large number of sample polypeptides bind non-specifically to antibody solid supports such as agarose or polymer beads. Attempts have been made to develop algorithms to help discriminate antibody-bound polypeptides from non-specific background binding, however, this remains challenging. The most thorough study on IP-MS published to date, reported successful identification of intended antibody targets (Marcon, E. et al., Nat Methods, 12, 725-731 (2015)). However, the method is not suitable to assess antibody specificity. Moreover, the authors did not provide evidence that IP-MS was useful to assess antibody cross-reactivity.

In summary, despite numerous attempts and large investments, academia and industry have failed to develop a widely applicable and cost-effective method for assessment of antibody specificity and sensitivity. Methods for antibody validation rely on subjective interpretation of data. It is therefore not feasible to establish robust and solid criteria for sensitivity and specificity. As a consequence, hundreds of millions, or even billions, of research grant funds are wasted yearly on experiments and research that yield poor and irreproducible results.

### SUMMARY OF THE INVENTION

The present invention addresses the shortcomings of current technology by implementing an innovative combination of sample polypeptide labeling, sample polypeptide separation, antibody array analysis and mass spectrometry (MS). Antibody array analysis of labeled and fractionated sample polypeptides allows independent detection of different targets bound by each of thousands of immobilised antibodies (Lund-Johansen, WO2009080370 A1). Parallel analysis by MS is facilitated by the use of an innovative approach for processing of labeled and fractionated polypeptides for MS analysis. Unexpectedly, the results obtained using the two methods are comparable to the extent that antibodies can be validated straightforwardly (and preferably automatically, e.g. using a computer algorithm) through correlating the antibody array data (or other binding agent array data) with the MS data in order to measure the similarity in the results. Importantly, the approach can yield results in a numerical format, allowing antibody sensitivity and specificity to be assessed objectively based on a numerical value. By allowing parallel and precise assessment of the specificity and sensitivity of thousands of antibodies in a single experiment, the instant invention represents a highly significant innovation that meets an urgent need for a more standardised and cost-effective approach to antibody validation.

Thus, in a first aspect, the present invention provides a method of analysing a mixture of polypeptides comprising the steps of:
(i) separating the polypeptides in the mixture into a plurality of fractions;
(ii) contacting a first aliquot of two or more of the fractions with a plurality of different binding agents attached to one or more solid supports and detecting the binding of the polypeptides to the binding agents in each fraction;
(iii) assessing the amino acid composition of the polypeptides in a second aliquot of said fractions by mass spectrometry; and
(iv) correlating the binding results detected in step (ii) and the mass spectrometry results from step (iii) to assess the specificity of the binding agents for a polypeptide of interest.

In a preferred embodiment, the method further comprises the steps of:
(v) determining one or more fractions which are enriched for a particular polypeptide of interest;
(vi) contacting the one or more fractions with a binding agent to said polypeptide of interest attached to one or more solid supports;
(vii) disrupting the binding agents of step (vi) from the associated polypeptides; and
(viii) contacting the released polypeptides with a plurality of binding agents attached to one or more solid supports and detecting the binding of the polypeptides to the binding agents.

Thus, the methods of the invention can be used for binding agent, e.g. antibody validation, for example to determine whether or not a particular binding agent (or a plurality or panel of different binding agents), can interact with a particular target protein (polypeptide), and, if they do bind, how specific or sensitive this binding interaction is.

Thus, alternatively viewed, the present invention provides methods of binding agent validation, or methods of determining or assessing the specificity and/or sensitivity of binding agents for a particular polypeptide of interest (target polypeptide).

Thus, the present invention provides a method involving the analysis of a mixture of polypeptides comprising the steps of:
(i) separating the polypeptides in the mixture into a plurality of fractions;
(ii) contacting a first aliquot of two or more of the fractions with a plurality of different binding agents attached to one or more solid supports and detecting the binding of the polypeptides to the binding agents in each fraction;
(iii) assessing the amino acid composition of the polypeptides in a second aliquot of said fractions by mass spectrometry;
(iv) correlating the binding results detected in step (ii) and the mass spectrometry results from step (iii) to assess properties of the binding agents or the polypeptides, for example for binding agent validation, or to assess specificity and/or sensitivity of the binding agent for a polypeptide.

Products for use in the methods of the invention are also provided.

As indicated above, the methods of the invention can be used to analyse several binding agents at the same time, i.e. the methods provide a multiplex assay, and is high throughput, quick and reliable. Such methods of the invention thus provide advantages over prior art methods. The methods of the invention can thus be used to assess the interaction of panels of binding agents (for example commercial binding agents such as antibodies), to a particular polypeptide of interest in order to validate the antibody, for example to determine specificity and/or sensitivity.

### DETAILED DESCRIPTION OF THE INVENTION

### Polypeptide Mixture

The method of the present invention may be carried out on any appropriate mixture of polypeptides. For example, the method may be carried out on one mixture (or one sample) of polypeptides, or alternatively carried out on more than one, or multiple, different mixtures or samples of polypeptides. The term “polypeptide” is used to cover any molecule comprising amino acid residues and includes proteins, peptides and oligopeptides.

The “polypeptide of interest” as referred to herein can be any appropriate polypeptide which can bind to a binding agent. Said polypeptide of interest thus includes, in a preferred embodiment, the polypeptide that a person carrying out the method of the present invention wishes to find a specific binding agent for, for example the polypeptide which is supposedly recognised by certain binding agents (such as antibodies). In this circumstance, information regarding the polypeptide is generally known beforehand, although the methods are not necessarily limited to embodiments where information regarding the polypeptide is known.

The mixtures are typically obtained from biological samples. Any appropriate biological sample can be used, examples of which would be readily determined by a person skilled in the art. In a preferred embodiment, the biological samples are selected from the list consisting of cell lysates or other cell samples, tissue extracts, tissue culture supernatants and a mixture thereof. In a preferred embodiment, the biological sample (or cell/tissue type) is selected from blood and blood products including plasma, serum and blood cells, bone marrow, mucus, lymph, ascites fluid, spinal fluid, biliary fluid, saliva, urine, extracts from brain, nerves and neural tracts, muscle, heart, liver, kidney, bladder and urinary tracts, spleen, pancreas, gastric tissue, bowel, biliary tissue, skin, thyroid gland, parathyroid gland, salivary glands, adrenal glands, mammary glands, gastric and intestinal mucosa, lymphatic tissue, mammary glands, adipose tissue, adrenal tissue, ovaries, uterus, blood and lymphatic vessels, endothelium, lung and respiratory tracts, prostate, testes, bone, lysates from cells originating from said organs, and lysates from bacteria, and yeast. The biological samples may be obtained from healthy subjects, diseased subjects or both (for example, where more than one mixture of polypeptides is being analysed). Where more than one mixture or multiple mixtures or two or more mixtures of polypeptides (samples) is being analysed, different samples, for example different sources of sample will generally be used. Preferred samples for such embodiments will be samples of different cell or tissue types. In other words, polypeptides from multiple different biological samples, for example multiple cell or tissue types, can be analysed.

The biological samples may comprise polypeptides in their native form or in their denatured form. The polypeptides will conveniently be present in solution before being subject to the separation step. As discussed below, through using the separation technique size exclusion chromatography, size fractionation may take place whilst retaining the polypeptides in their native form and such separation methods are preferred when native proteins are to be analysed. By contrast, gel electrophoresis generally requires that the polypeptides are denatured before and during the fractionation process.

### Labeling of the Polypeptides

In a preferred embodiment the methods of the present invention further comprise attaching at least one label to the polypeptides present in the mixture of polypeptides or the one or more further mixtures of polypeptides. Appropriate labels which allow detection would be well known to a person skilled in the art. For example, the label may be directly detectable or may be indirectly detectable (for example requiring an interaction with a second or another directly detectable moiety, for example a fluorescent moiety/dye, or an isotope before detection can take place). The label may be a reporter molecule.

Labelling of the polypeptides present in the mixture of polypeptides typically takes place before the detection of binding occurs in step (ii) as such labelling can be used in the detection step. Preferably, the step of attaching the label or labels to the polypeptides present in the mixture of polypeptides or the one or more further mixtures of polypeptides is carried out prior to step (i) or after step (i), most preferably prior to step (i). Alternatively, the labelling can be carried out during step (ii) but prior to the detection step.

When more than one label is used, it is preferable that a different label is attached to the mixture of polypeptides in each fraction, or each fraction of the one or more further mixtures of polypeptides. More preferably, a different label is attached to each mixture of polypeptides (for example to the polypeptides of each different cell type), where more than one mixture of polypeptides is analysed.

However, it is also possible that more than one label is attached to the polypeptides present in the same fraction. This may for example be carried out by having a different label attached to each mixture of polypeptides (e.g. a different label for each different cell type) and then combining polypeptides from two or more of these mixtures in the same fraction e.g. after the separation step. Alternatively more than one label can be attached indiscriminately to all fractions analysed. Such multiple labels could label different parts of a polypeptide which may then add complexity to the signature for a particular polypeptide and could be useful for determining whether or not a binding agent has bound to the polypeptide of interest. By way of example, cysteines in a polypeptide may be labelled with biotin-maleimide and amines labelled with N-hydroxysuccinimido (NHS) digoxigenin, or conversely cysteines in a polypeptide may be labelled with digoxigenin-maleimide and amines labelled with NHS biotin.

In a preferred embodiment, the or each label comprises a hapten (such as biotin or digoxigenin, preferably biotin), a fluorescent dye, a luminescent dye, a radioactive isotope, a non-radioactive (stable) isotope, or a mixture thereof. In the most preferred embodiment, the label is biotin, which can be detected upon binding to an appropriately labeled streptavidin containing molecule, for example a fluorescent streptavidin molecule such as a streptavidin-phycoerythrin conjugate. Where more than one label is used, it is preferable to use more than one hapten (such as the combination of biotin and digoxigenin). In an alternative embodiment, the multiple labeling may be in the form of more than one non-radioactive (stable) isotope.

There are many commonly known methods of attaching a label to polypeptide and any of these may be used to prepare the labelled polypeptides for use in the present invention. In a preferred embodiment, the label is attached to the polypeptides present in the mixture of polypeptides via a chemically reactive group. In a preferred embodiment, the label is attached to the mixture of polypeptides via a peptide, a polypeptide, an oligonucleotide, or an enzyme substrate. When the label is biotin, biotinylation methods are well known in the art, such as, for example, primary amine or sulfhydryl biotinylation using for example an amine- or a thiol-reactive derivative of biotin.

Such labels can conveniently be used in step (ii) of the method in order to detect the binding of the polypeptides to the binding agents.

Alternatively, the binding between a binding agent and a polypeptide is detected by a label free system, preferably, surface plasmon resonance or magnetic resonance.

Separation of a Polypeptide Mixture into a Plurality of Fractions

The separation step (i) wherein a mixture of polypeptides is separated into a plurality of fractions provides a way of reducing the number of different polypeptides present within each fraction so that, if binding of a polypeptide to a binding agent is detected in step (ii), there is an increased likelihood that this binding agent is specific for the polypeptide of interest. For this reason, a high number of fractions is preferable. In a preferred embodiment step (i) comprises separating the polypeptides in the mixture into at least 3, 4, 5, 6, 7, 8, 9, 10, 11, 12, 14, 16, 18, 20, 22, 24, 48, 96 or 200 fractions, preferably at least 5, 12, 24, 48 or 96 fractions (i.e. 5 or more, 12 or more, 24 or more, 48 or more, or 96 or more fractions). However, multiple 96-well plates can also be used in the methods (e.g. forming up to or at least 192, 288, 384, 480, 576, 672, 768, 864 or 960 fractions). The number of fractions obtained in the separation step may thus be between 3 and 2000, preferably between 3 and 1000, more preferably between 4 or 5 and 500, more preferably between 10 and 200 or 300 fractions. As the methods of the invention can conveniently be carried out in 96 well plates, preferred numbers of fractions are multiples of 12 (for example 12, 24, 36, 48, 60, 72, 84 or 96, etc.) so that the plurality of fractions occupies one or more complete rows of the plate. Alternatively, preferred numbers of fractions are multiples of 8 (for example 8, 16, 24, 32, 40, 48, 56, 64, 72, 80, 88 or 96, etc.) so that the plurality of fractions occupies one or more complete columns of the plate.

The present invention may utilise a wide range of types of fractionation, providing that the fractionation results in a reduced number of different polypeptides present within each fraction compared to the starting mixture. Conveniently, in step (i) of the method of the invention, polypeptides can be separated into a plurality of fractions on the basis of one or more physical parameters of the polypeptides. Fractionation on the basis of one or more of the following physical parameters may, for example, be used: differential mass, acidity, basicity, charge, hydrophobicity and binding to different affinity ligands. In order to fractionate on the basis of such parameters any appropriate technique may be used. For example, the following techniques may be used: gel electrophoresis (SDS PAGE), size exclusion chromatography, liquid chromatography, dialysis, filtration, ion exchange separation (ion exchange chromatography) and iso-electric focusing. Size exclusion chromatography (SEC), ion exchange chromatography, affinity chromatography or gel electrophoresis are preferred techniques.

Methods of affinity chromatography would be well known to a person skilled in the art. Examples of protein-binding reagents that are commonly used in this technique are heparin, metal ions, glutathione, lectins, recombinant proteins and antibodies.

Size exclusion chromatography can be used to separate native polypeptides and is widely used as a first dimension in identification of multi-molecular complexes. Due to the low resolution of size exclusion chromatography, the method can be usefully combined with a second separation method. An appropriate second method is SDS-PAGE (gel electrophoresis), which separates denatured polypeptides by their size.

In some alternative embodiments, sub-cellular location can be used as the basis for separating the mixture of polypeptides, for example fractionation of a cell lysate/homogenate can be used to separate a sample into different sub-cellular fractions (sub-cellular fractionation). Sub-cellular fractionation can be used to obtain information about the distribution of molecules in different cellular compartments. For example, membrane polypeptides can be isolated from other cellular components. Such polypeptides generally have hydrophobic domains and remain associated with lipids when a cell is disrupted in the absence of detergents or in the presence of low levels of detergents. Other cell compartments that can be isolated as separate components include the nucleus, organelles and the cytoplasm. Thus, a cell sample or extract with a complex mixture of polypeptides can be separated into a plurality of fractions with a reduced number of different polypeptides in each fraction by a relatively simple fractionation into a limited number of sub-cellular fractions. The data disclosed herein show that sub-cellular fractionation is a highly useful technique for use in the present invention. Sub-cellular fractionation may preferably be combined with separation on the basis of a different parameter, for example on the basis of size.

Indeed, in some embodiments it is preferable that fractionation takes place on the basis of more than one parameter, such as the combination of size and subcellular location or combinations of other parameters as discussed above or elsewhere herein. Fractionation on the basis of more than one parameter (for example, at least 2, 3, 4, 5, 6, 7, 8, 9 or 10 parameters) can provide further dimensions of analysis with respect to correlation step (iv), as discussed in further detail in the comparative analysis section below. The use of more than one parameter can add complexity to the total data obtained (to the signature or data signature) for a particular polypeptide and this additional complexity can sometimes be advantageous in identifying whether a binding agent binds specifically to a polypeptide or the sensitivity of binding. In general, the more fractions that are analysed, the more unique is the signature.

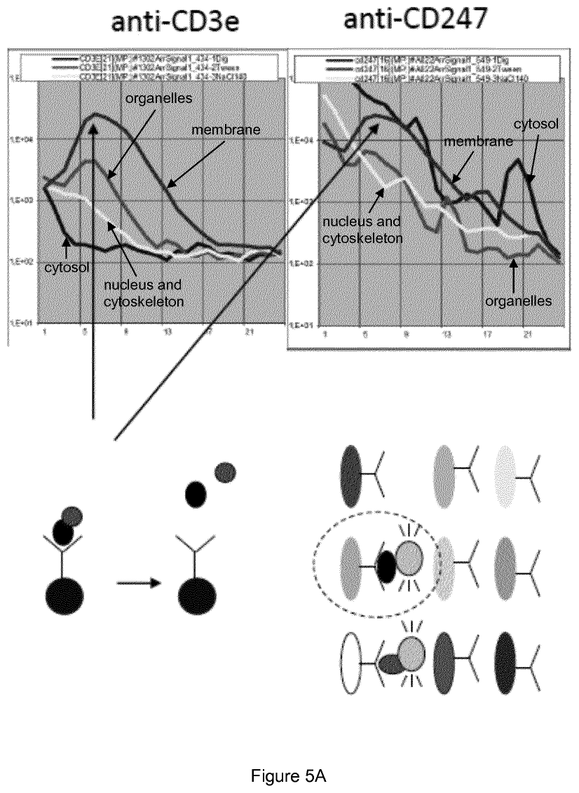

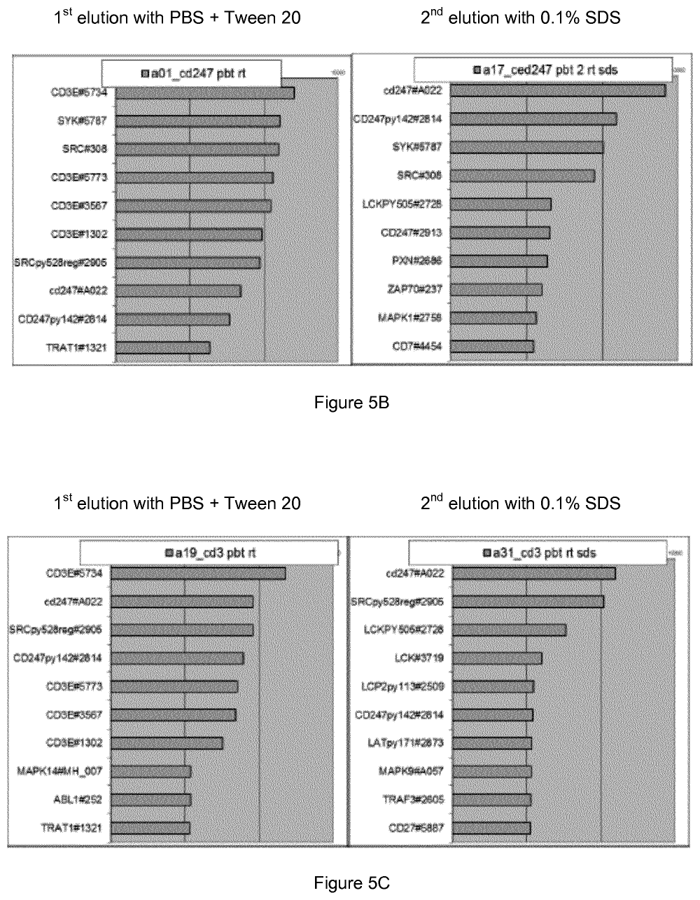

Preferably the number of parameters is between 1 and 20, more preferably between 1 and 10, more preferably between 2 and 5. The level of fractionation discussed above relates to the number of fractions that form after fractionation on the basis of all intended parameters. For example, with respect to the combination of size and subcellular location parameters given above, 4 subcellular locations and 24 size fractions results in (4×24=) 96 fractions in total. Such fractionation is exemplified in FIG. 5 and Example 2.

In this regard, analysing more than one mixture of polypeptides may form one of the parameters in itself, and this is particularly preferred. More preferably, at least one parameter is in the form of different samples, for example cell types. Preferably, at least 2, 3, 4, 5, 6, 7, 8, 9, 10, 12, 14, 16, 18 or 20 different samples (e.g. cell types) are analysed. Preferably, separation is carried out on basis of cell type and of size.

The greater the level of separation or the greater number of parameters (e.g. cell types) used, the more complex the data signature obtained at step (iii) of the present invention after MS. Therefore a greater level of separation or a greater number of parameters (e.g. cell types) used leads to a more precise correlation in step (iv) with regard to the specificity and/or sensitivity of a particular binding agent. For example, if two or more mixtures of polypeptides from different cell types are used, then a second parameter in the form of the relative abundance of the polypeptide in the cell types, can be analysed.

In appropriate embodiments, methods of sub-cellular fractionation to allow isolation or separation of polypeptides into different sub-cellular components based on cellular location (for example one or more of membrane, cytoplasm, nucleus, organelles) are known to a person skilled in the art.

Typically separation step (i) would result in one or more master plates containing all of the fractions. Aliquots from a plurality (two or more) of the fractions would then be taken from these plates for both binding analysis (step (ii)) and mass spectrometry (MS) analysis (step (iii)), and the transfer could be made to replicate plates of the same format as the master plates in order to allow easy correlation between the replicate plates and the master plates. Equally, in embodiments described elsewhere herein where the solid supports are planar supports, the replicate aliquots could be transferred to appropriate areas of appropriate solid supports for the binding analysis of step (ii) and the MS analysis of step (iii) to take place. Such replicate aliquots taken from a master plate would therefore contain the same mixture of polypeptides which would be subjected to both the binding analysis of step (ii) and the MS analysis of step (iii). Thus, for each fraction of the master plate which is to be analysed, in general two replicate aliquots are taken from the master plate and individually subjected to the two different analysis methods. These two replicate aliquots are sometimes referred to herein as first and second aliquots. Preferred numbers of fractions that are taken for both binding analysis (step (ii)) and mass spectrometry (MS) analysis (step (iii)) are described above.

In a preferred embodiment, a liquid handling robot is used in order to transfer aliquots from the master plates to the replicate plates or other replicate solid supports for enhanced reproducibility. For the purposes of transferring aliquots of the fractions (for example from a master plate to a replicate plate) it is preferable that the fractions are in liquid form (in other words, the polypeptides are dissolved within a liquid). There are many separation techniques known in the art that would lead to liquid fractions and any of these may be used. Exemplary methods include gel electrophoresis using for example a GELFREE® 8100 instrument, liquid chromatography and size exclusion chromatography.

### Binding Agent

In preferred embodiments, the binding agent is an antibody or an antigen binding fragment thereof. In such embodiments any type of antigen binding fragment could be used, examples of which would be well known to a person skilled in the art. However, the skilled person would fully appreciate that the methods of the present invention would be equally effective in assessing the specificity of non-antibody binding agents. Again a person skilled in the art would readily be able to identify other types of binding agent which could be used, the main requirement being that such binding agents are capable of binding specifically to polypeptides (referred to herein as target polypeptides or polypeptides of interest). Thus, it is generally preferred that any alternative (non-antibody) binding agent must have the same degree of binding specificity as an antibody when it binds specifically to a polypeptide or antigen.

In preferred embodiments, the binding agents used bind to only one target polypeptide. However, binding agents which bind to 2, 3, 4 or 5 target polypeptides can also be used.

In other embodiments a binding agent (for example an antibody or non-antibody) which binds to between 2 and 20 target molecules in a prokaryotic or eukaryotic cell lysate would be a suitable binding agent but a binding agent that binds over 100 target molecules in such a cell lysate would not be a suitable binding agent. This is particularly appropriate for binding agents which can bind to protein motifs.

Thus, alternatively, some binding agents have the ability to bind to motifs that are present in many proteins, for example there are binding agents (for example antibodies) that can bind to post-translational modifications such as phosphotyrosine and can therefore bind many proteins. Such binding agents may equally be useful in the present invention, for example to enrich for modified proteins. Thus, in one embodiment a binding agent that can bind to (is specific for) one to three specific binding motifs (such as those comprising a phosphorylated amino acid in the polypeptide of interest) in a prokaryotic or eukaryotic cell lysate would also be a suitable binding agent.

In addition, the binding agents useful in the present invention generally have a binding affinity for their target of less than 1 μM under physiological conditions, preferably less than 100 nM.

Thus, in some embodiments, a different (non-antibody) binding agent is used. The following are examples of such binding agents: aptamers (or other nucleic acid based binding agents), affibodies, polypeptides, peptides, oligonucleotides, T-cell receptors, MHC molecules.

The term “binding specificity” as used herein refers to the ability of a binding agent to bind to one polypeptide (or protein motif) specifically. A binding agent that binds to only one polypeptide is considered monospecific. For the purposes of the present invention, binding specificity is considered to be the same as binding selectivity. It is known in the art that specificity is a statistical measure which is also known as the “true negative rate”, and measures the proportion of negatives that are correctly identified as such. In this context, a high level of specificity means that a low number of false positives (i.e. the binding agent binding to something other than the polypeptide of interest) would be seen.

The term “binding sensitivity” as used herein relates to how strongly a binding agent binds to a polypeptide (or protein motif). It will be appreciated that some binding agents may be monospecific (i.e. bind to only one polypeptide) but have a low sensitivity (i.e. bind to that polypeptide with a low affinity/low strength), and, by contrast, some binding agents may be very sensitive but not bind specifically. The method of the present invention is able to determine both the specificity and the sensitivity of a binding agent with respect to a particular polypeptide of interest. It is known in the art that sensitivity is a statistical measure which is also known as the “true positive rate” or “probability of detection” and measures the proportion of positives that are correctly identified as such. In this context, a high level of sensitivity means that there is a high probability that a binding agent will bind to a polypeptide of interest if present in a sample (such as a mixture of polypeptides).

### Binding Agents Attached to One or More Solid Supports

In a preferred embodiment, the binding agents that are attached or immobilised to one or more solid supports are attached on the surface of a planar substrate (for example on the surface of a membrane or in the well of a multiwall plate). The substrate (for example the planar substrate) may alternatively, have three-dimensional (for example raised or, alternatively, dimpled or lowered) structures on its surface in some embodiments, for example to provide discrete areas for attachment of the binding agents. The binding agents may be arranged in any appropriate configuration so as to allow contact with the polypeptides in the various fractions and the assessment of binding. For example, the arrangement may take the form of an array of spots (or wells), each spot (or well) comprising multiple copies of the same binding agent (and different spots (or wells) comprising different binding agents). The identity of the binding agent on the array can be determined by their location on the array as is well known in the art for array based techniques.

In use in the methods of the present invention, the mixture of polypeptides is separated as described elsewhere herein and then the array is contacted with the first fraction from the mixture. Unbound sample is then preferably removed from the array (for example, by washing) and the array is then examined at each area (for example a spot or well) where a binding agent is attached to determine whether any polypeptides are bound at the spot (or well) and hence to detect whether there is any binding of polypeptides from the fraction to binding agents on the array. Once each area (e.g. spot) is analysed, the results can be compiled in a similar manner to that described elsewhere herein. A second array is then provided which is contacted with the second fraction of the sample and the process is repeated until all of the desired sample fractions have been analysed. The second and third arrays, etc., can be provided on the same or different solid support as the first array, provided that the arrays are spatially separated from each other.

In an alternative and preferred embodiment, the binding agents are attached to or immobilised on a plurality of particles, each particle having attached thereon multiple copies of the same binding agent. The particle may be in the form of a bead, a microsphere, preferably a latex microsphere, a quantum dot or a nanoparticle, such as a nanocrystal. The particles may be magnetic to facilitate pelleting with magnets, or non-magnetic for pelleting by centrifugation or filtration devices. In such embodiments the solid supports are particles.

By “same binding agent” it is understood that any two copies of the binding agent are specific for or selected for binding to the same polypeptide. For example, in the case of a polyclonal antibody, such antibodies will consist of a plurality of antibodies with different amino acid sequences. They have been selected for binding to the same polypeptide, but the solid support will be covered with polypeptides that have many alternative compositions. Alternatively, for example in the case of a monoclonal antibody, any two copies of the same binding agent may be indistinguishable with respect to binding reactivity and/or structure, for example the particle or area of the array contains multiple copies of the same binding agent, for example the same antibody. In this case, the same binding agents may have the same amino acid or nucleic acid sequence as each other.

In use, clearly more than one particle with a particular binding agent attached is likely to be required for binding to polypeptides to be detected. In other words multiple particles with a particular binding agent attached are likely to be required. Multiple particles that have attached thereon multiple copies of the same binding agent form a set.

In a preferred embodiment, a first set of particles having attached thereon multiple copies of the same binding agent have a different detectable feature from a further set of particles having multiple copies of a binding agent that is different to the binding agent attached to the first set of particles. Generally when the particles are prepared, it is known which binding agent is attached to the particles with a particular detectable feature. In this way, during the methods of the invention, the detectable feature may then also be used in order to determine the nature of the binding agent attached thereon. The detectable feature may need to be applied to the particles, through for example a labelling step. However, it is also possible that the particle has inherent properties that allow one type of particle to be distinguished from another type. Examples of this form of particle include quantum dots and nanocrystals that can have a wide range of fluorescence emission maxima.

The detectable feature may be based on fluorescence, isotopes, for example radioactive isotopes or non-radioactive (stable) isotopes, luminescence, size or acoustic properties. Each different detectable feature in effect takes the form of a code, and different binding agents can be attached to particles with different codes.

In a preferred embodiment, the detectable feature is in the form of at least one type of dye molecule, preferably a type of fluorescence dye, attached to the particle, preferably at least three types of dye molecules attached to the particle. More preferably the or each type of dye molecule is selected from the list consisting (or comprising) of (i) a dye molecule having an absorption maximum of 405 nm and an emission maximum of between 420 and 450 nm; (ii) a dye molecule having an absorption maximum of 405 nm and an emission maximum of greater than 500 nm; (iii) a dye molecule having an absorption maximum of 488 nm and an emission maximum of between 520 and 530 nm; (iv) a dye molecule having an absorption maximum of 632 nm and an emission maximum of between 650 and 670 nm and (v) a dye molecule having an absorption maximum of 632 nm and an emission maximum greater than 670 nm. More preferably the or each type of dye molecule is selected from the list consisting (or comprising) of Alexa 488, Alexa 647, Pacific Blue, Pacific Orange and Cy7.

The use of more than one type of dye as described above and the use of various concentrations of the dyes, and various combinations of the concentrations of dyes, allows one to set a vast array of differently colour codes that can be distinguished from one another using, for example, flow cytometry. This, in turn, allows the analysis of numerous varying binding agents within each fraction as a different binding agent can be attached to particles with a different code. The manufacture and use of these labelled particles (e.g. particles with addressable fluorescent bar codes) is known in the art and described in International patent publication WO 2007/008084.

The binding agents can be attached to the solid support by any appropriate means which would be well known to a skilled person. In a preferred embodiment, the binding agents are attached to the solid support via an appropriate affinity coupling, examples of which would be well known in the art. In particular, the affinity coupling can be via immunoglobulin-binding affinity reagents such as Protein G, protein A, Protein NG, Protein L, anti-immunoglobulin antibodies or fragments thereof. Alternatively, the binding agents may be modified with a hapten such as biotin or digoxigenin, or peptide, or DNA motifs and bound to the solid supports (for example particles) via binding agents specific for the modifications.

### Analysis of Binding Between Polypeptide and Binding Agent

When the method of the present invention is carried out on one or more planar substrates as solid supports, analysis or detection of binding of polypeptides to binding agents would generally be carried out through the use of a plate reader, an array scanner or any other suitable equipment. As described above, the location of a spot (area or well) on a planar substrate can provide information regarding the particular fraction of the mixture which is being tested and/or the nature of the binding agent present. When a label is attached to the polypeptides, then such a label can be detected (either directly or indirectly, as discussed above), and the intensity of the signal detected would generally correlate to the extent of binding that has taken place between a polypeptide and a binding agent with respect to a particular fraction and/or the nature of the binding agent. Generally, relative signals would be determined across a series of fractions within the same sample and/or across fractions obtained in different samples (e.g. two or more cell types, or two or more subcellular compartments, for example to determine relative abundance of polypeptides in two or more cell types, or two or more subcellular compartments).

When the method of the present invention is carried out on a plurality of particles as solid supports, a flow cytometer is generally used to analyse or detect binding of polypeptides to binding agents. When both a detectable feature (e.g. a detectable code) is used with respect to the particles and the polypeptides are labelled, it is important that the label and the detectable feature are distinguishable so that a flow cytometer is able to determine both the nature of the binding agent attached to the particle (based on the detectable feature) and whether (and to what extent) polypeptides are bound to the binding agents attached to the particle (based on the label), for each particle analysed. Raw flow cytometry data (typically in FCS format) are analysed using software that allows identification of microsphere subsets on the basis of their detectable features (such as colour codes or addressable bar codes) (Stuchly, J. et al., Cytometry. Part A, 81, 120-129 (2012)) and the amount of label associated with each particle.

Alternatively, when the method of the present invention is carried out on a plurality of particles as solid supports, mass cytometry (measured by a mass cytomer, which is a hybrid between a flow cytometer and a mass spectrometer) may also be used. Here, the detectable feature present on the particles (and optionally the polypeptides in the sample) would generally be one or more stable isotopes, and in this regard one can use up to 40 different isotopes as labels with no overlap in spectra. Analysis of these particles would be carried out using mass spectrometry. Methods of carrying out mass cytometry are well known to a person skilled in the art.

It is possible that, when more than one set of particles with binding agents attached thereon, as described above, is in contact with a fraction of polypeptides, one or more binding agents become detached from their respective particles and then become attached to a particle with a detectable feature relating to a binding agent that is specific for another polypeptide compared to the newly attached binding agent. This in turn could lead to false positives (where the binding results indicate that a particular binding agent has bound to the polypeptide of interest when this is not the case). In order to minimise this from happening, it is preferable that contact step (ii) is carried out in the presence of a non-functional binding agent, such as non-immune IgG antibody. The non-functional binding agent is preferably present at a concentration far greater than the predicted concentration of the binding agents released from the particles, for example at a concentration that is more than 100 times greater than the predicted concentration of the binding agents released from the particles. The presence of this non-functional binding agent would effectively dilute the concentration of binding agents released from the particles and therefore reduce the likelihood of those particles becoming attached to a particle with a detectable feature relating to binding agent that is specific for another polypeptide compared to the newly attached binding agent.

The preferred output of the detection step is a spread sheet-compatible file (e.g. a text file) with the detectable feature of the particle (and hence an identifier/particle identifier, for the particular binding agent which is attached to the particle) and the corresponding values for the intensity of the label (where a label is used), e.g. fluorescent signal intensity, in each fraction which is assessed. The data file (e.g. text file), which can be referred to as the binding assay/array data file or the binding agent data file, with results from such binding agent array analysis (e.g. antibody array analysis) contains identifiers for each binding agent and their intended targets, numerical values for the relative binding signal intensity of polypeptide targets bound to a particular binding agent in the fractions. In other words these numerical values reflect the relative abundance of polypeptide targets (e.g. the antibody or binding agent targets) in the fractions. These (i.e. the series of numbers from a set of fractions) can be referred to as binding chromatograms (or binding agent-target chromatograms or antibody-target chromatograms (in cases where the binding agent is an antibody)). The data files can be obtained by any appropriate means which will be well known, for example depending on the method and instrumentation used to collect the data. Thus, for example when the data is flow cytometry data these data can be processed using for example R script analysis in order to obtain a set of numerical data for further processing and analysis or for correlation.

By “relative” it is meant that the value of the binding signal intensity within a particular fraction is reflected as a proportion of all of the values from either a series of fractions or all of the fractions combined. For example, if the relative binding signal intensity (or relative abundance) for a particular fraction was 0.5 and the total relative binding signal intensity for either a series of fractions or all of the fractions combined was 1, it can be concluded that half of the binding events have taken place in that particular fraction. Binding signal intensity is generally analysed in the form of a median fluorescence intensity (MFI), the median value taken from the signal intensities of preferably at least 30 particles. The binding signal intensity values are generally normalised, for example by subtracting the signal detected from particles with no binding agent attached from the binding signal intensity values with binding agent present, before analysis of the binding results is carried out. Of course, this median value analysis and normalisation process can be carried out regardless of whether the binding signal intensity is measured by fluorescence or by some other means.

### Mass Spectrometry

Mass spectrometry is used in order to assess the relative abundance of polypeptides contained in each fraction and their amino acid composition. In a preferred embodiment, the amino acid sequence of the polypeptides is determined.

The person skilled in the art is readily aware of how to prepare samples comprising a mixture of polypeptides for mass spectrometry analysis. For example, after separation step (i), it is possible that polypeptide mixtures will be in the presence of salts and/or detergents that are incompatible with MS analysis, and so sample preparation will generally involve the removal of such components and purification of these polypeptides, e.g. by appropriate washing steps.

Where separation step (i) results in liquid fractions, in the aliquots that are to undergo MS analysis, polypeptides may be attached or otherwise immobilized onto an appropriate solid phase as part of the sample preparation. It is desirable that all polypeptides in the fraction be attached to the solid phase and appropriate methods of doing this would be well known to a skilled person. Such attachment is preferably indiscriminate, i.e. attachment would take place to the same degree with respect to all polypeptides in the fraction. Thus, attachment of the polypeptides to the solid phase may be carried out using chemical methods, or using a general affinity reagent, e.g. via affinity coupling. For example, when the polypeptides are labeled, it is preferred to use the polypeptide label or labels described above to capture the polypeptides onto a solid phase. For example, where the polypeptides are biotinylated, streptavidin covalently coupled to a solid phase may be used in order to carry out the attachment process. In preferred embodiments, the label used for detection in the binding agent array analysis may also be used to carry out the attachment for the MS analysis, e.g. via a biotin-streptavidin link.

Preferred solid phases include particles, preferably particles comprising polysaccharides such as agarose, or polymers such as monodisperse latex microspheres. It is however appreciated that attachment may take place on a planar surface also, such as the planar surfaces discussed above. In a preferred embodiment, the particles are processed in microwell plates using liquid handling robots to enhance reproducibility. The particles may be magnetic to facilitate pelleting with magnets, or non-magnetic for pelleting by centrifugation or filtration devices. In the most preferred embodiment, streptavidin beads are used in combination with biotinylated polypeptide mixtures (although of course other pairs of affinity partners may be used).

Polypeptides bound to a solid-phase are digested to yield soluble peptides prior to MS analysis. The polypeptides may be digested while bound to the solid-phase (e.g. on-bead digestion). Alternatively, the polypeptides may be released from the solid-phase and then digested. In both cases, the digestion step yields a complex mixture of soluble peptides. Appropriate means of digestion would be well known to a person skilled in the art, for example with a proteolytic enzyme to generate peptides suitable for MS analysis. For example, trypsin can conveniently be used. Such digestion steps provide a means for carrying out the disrupting step (vii) in embodiments where said disrupting step is followed by an MS step. In a preferred embodiment, the polypeptides are further purified by hydrophobic interaction chromatography (HIC) prior to analysis.

Typically mass spectrometry analysis is carried out using a bottom-up proteomics approach, where polypeptides are digested into fragments (peptide fragments) before processing, and then the data (e.g. the amino acid sequence) of the fragments are used to determine the nature of the polypeptides present in a fraction. As discussed above, digestion may be carried out using any techniques commonly known in the art, such as trypsin digestion.

However, it will be appreciated that a top-down proteomics approach could be used also, where the processing of intact polypeptides and fragments thereof is carried out. Such a top-down approach would still involve release of the polypeptides from the solid-phase prior to MS analysis.

In a preferred embodiment, liquid chromatography mass spectrometry is used. Typically peptides are solubilized using, for example, formic acid, and then loaded onto a nano-liquid chromatography column interfaced directly into a mass spectrometer. Liquid chromatography mass spectrometry may be used in combination with tandem mass spectrometry (also known as MS/MS or MS²). Briefly, MS/MS, as known in the art, is where two stages of MS are carried out, the first stage to detect the mass to charge ratio of a certain polypeptide (often referred to as “MS1”) and the second stage to analyse the amino acid composition after fragmentation.

In other embodiments it is not necessary to use a solid phase as part of the MS analysis. For example, other techniques such as gel trypsin digestion or filter-aided sample preparation (FASP) may be used. In FASP, the separation is based on the larger size of proteins compared to MS-incompatible components such as salts and detergents.

In a preferred embodiment of the methods of the invention, as described elsewhere herein, cellular proteins are labelled with stable (e.g. non-radioactive) isotopes by metabolic labelling, e.g. using SILAC (stable isotope labelling with amino acids in culture). This step serves as means to trace the peptides detected by MS to a particular cell type. Those skilled in the art will know how to use metabolic labelling and analyse the MS data. The use of this technique also allows multiple samples (e.g. up to three samples) to be run simultaneously in the MS machine.

The preferred data file produced after MS (the MS data file) contains numerical values for the relative abundance of thousands of proteins in the fractions. The series of numbers for a polypeptide of interest is sometimes referred to herein as the MS-chromatogram. The data files can be obtained by any appropriate means which will be well known, for example depending on the method and instrumentation used to collect the data. Thus, for example when the data is MS data these data can be processed using for example MaxQuant analysis in order to identify proteins and to obtain a set of numerical data for further processing and analysis or for correlation.

As with the relative binding signal above, “relative” here means that the value of the abundance within a particular fraction for a particular polypeptide of interest is reflected as a proportion of all of the values from either a series of fractions or all of the fractions combined (for that particular polypeptide). For example, if the relative abundance for a particular fraction was 0.5 and the total relative abundance for either a series of fractions or all of the fractions combined was 1, it can be concluded that half of the polypeptide of interest from the mixture of polypeptides is in that particular fraction.

### Parallel Binding (Binding Agent) and Mass Spectrometry Analysis

The methods of the present invention advantageously involve parallel analysis or assessment of binding results (i.e. binding of polypeptides to binding agents) to MS results. By “parallel”, it is understood that an identical or representative (but separate) aliquot (i.e. an aliquot containing identical or representative polypeptides) from the same fraction obtained in step (i) of the method is analysed with respect to binding of polypeptides to a binding agent as described in step (ii) and with respect mass spectrometry as described in step (iii), and results are compared (correlated) as described in step (iv) and in further detail below. It is, however, appreciated that, in practice, the binding analysis or assessment (detection) of step (ii) need not be carried out at the same time as the MS analysis or assessment of step (iii) and indeed step (iii) may be carried out before step (ii) or vice versa. In other words the steps can be carried out in any appropriate order.

Comparative Analysis (Correlation) of Binding Results with the Mass Spectrometry Results

Once results from the binding array (binding results), preferably from an antibody array, and mass spectrometry results have been obtained, these results are correlated in order to for example assess the specificity of the binding agents for a polypeptide of interest, as described in step (iv) of the method of the invention. These results are generally presented in data files, for example text files or in the form of spreadsheets, with identifiers (e.g. particle identifiers (in embodiments where particles/beads are used), binding agent identifiers (e.g. antibody identifiers) in relation to a particular binding agent (e.g. antibody) and/or protein identifiers in relation to a target protein/polypeptide of interest for the array data, or protein identifiers in relation to a particular polypeptide of interest for the MS data) and corresponding numerical values for signal intensity measured in a series of fractions which have undergone parallel binding agent array analysis (e.g. antibody array analysis) and mass spectrometry analysis. For the binding array analysis (array analysis), results would be presented in such files with respect to each binding agent analysed separately. The binding array data/results are then correlated with the MS data/results. The correlation can simply be the correlation between binding array results (e.g. binding array signals) in a chosen set of fractions (e.g. based on the fractions which have the best resolved proteins), e.g. fractions 1 to 12, fractions 2 to 12, fractions 3 to 12 or fractions 4 to 12 in a 12 fraction experiment), and the MS results (e.g. the MS signals) in the same fractions. This correlation can also be referred to as the specificity index and can conveniently be measured as a proportion or a percentage.

If a particular binding agent (for example an antibody) binds specifically to a polypeptide of interest, the binding array data (binding array signal), for example in the form of a binding chromatogram as discussed above, is expected to overlap or match closely with the MS data for the polypeptide of interest (intended target polypeptide), for example in the form of an MS chromatogram discussed above. Thus, the correlation step (iv) can be carried out by measuring the overlap between the binding results of step (ii) and the MS results of step (iii), for example by specifically measuring the overlap between the binding chromatogram and the MS chromatogram.

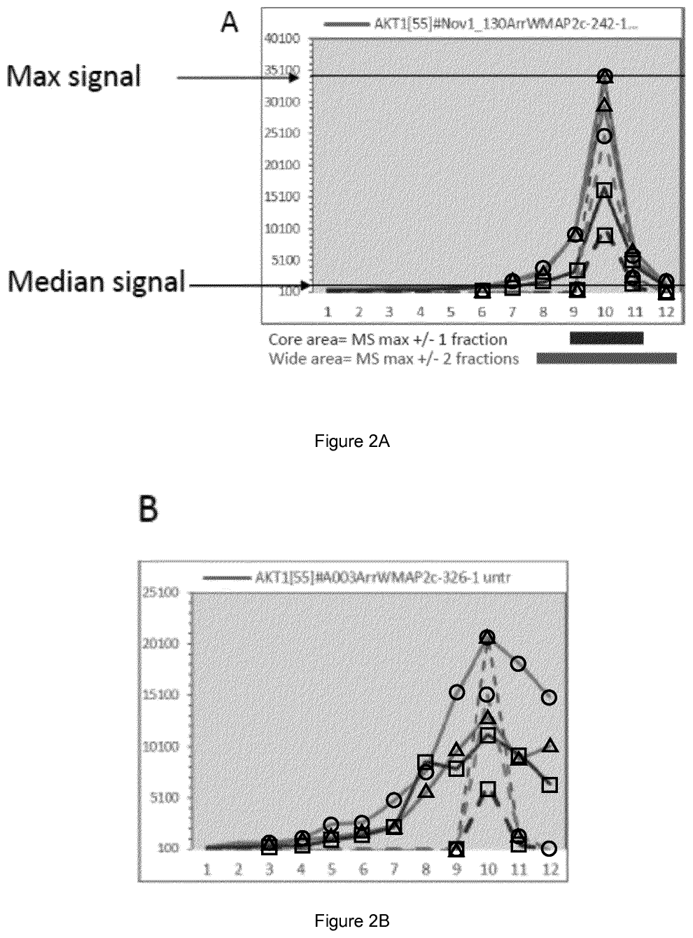

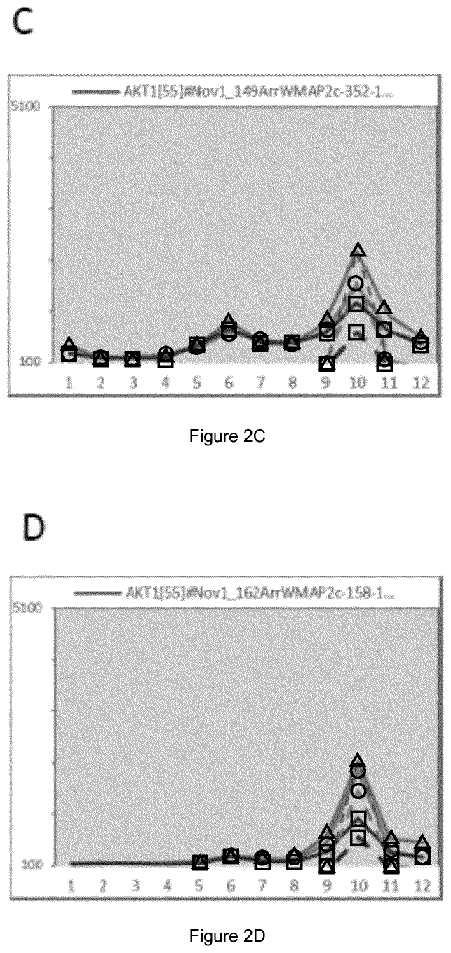

A person skilled in the art would readily know how to correlate the sets of numerical data, in particular any relevant two sets of numerical data (i.e. a set of binding array data for a particular binding agent which is supposed to bind to a target polypeptide of interest, with a set of MS data for that same target polypeptide). For example, appropriate algorithms can be designed to measure the correlation or overlap (or otherwise assess the fit or similarity) between the two respective sets of data or chromatograms. Indeed, several methods for analysing chromatograms are described in the scientific literature and any of these may be used. For example, Scott and co-workers (Scott, N. E. et al., J Proteomics, 118, 112-129 (2015)) describe a general algorithm for analysing results obtained by MS analysis of a series of fractions obtained by size exclusion chromatography (SEC) or subcellular fractionation. The chromatograms corresponding to polypeptides that have been separated by one dimensional gel electrophoresis (1DGE) are expected to have a unimodal Gaussian (symmetric) shape, as shown in FIG. 2.

In order to correlate or specifically determine the level of overlap between binding results of step (ii) and the MS results of step (iii), some form of data processing (which can also be referred to herein as data manipulation) may be necessary in order to make direct comparisons between the binding results and the MS results. The skilled person can straightforwardly determine how such data processing can be carried out.

Scaling can be a useful technique for use in the methods of the present invention in order to process the binding results and the MS results. Such a technique is particularly useful for preparing graphical displays of the data. For example, either the binding results can be upscaled or downscaled so that they can be compared against the MS results, or conversely the MS results can be upscaled or downscaled so that they can be compared against the binding results. Upscaling means to increase all of the values in a data set (such as the binding results) by the same factor, so that the difference between one value and another is maintained in relative terms. Conversely, downscaling means to decrease all of the values in a data set by the same factor, again so that the difference between one value and another is maintained in relative terms. It is also possible to upscale or downscale both the binding results and the MS results.

There are a number of ways in which a skilled person can determine the extent to which either the binding results or the MS results are upscaled or downscaled in order to usefully process the results (and indeed some algorithms will perform these steps automatically). For example, upscaling or downscaling may take place so that the mean binding signal from the binding results (with respect to a series of fractions) matches the mean relative abundance from the MS results. Alternatively upscaling or downscaling may take place so that the median binding signal from the binding results (with respect to a series of fractions) matches the median relative abundance from the MS results. It is important to note here that, in order to carry out this scaling, the binding results and the MS results do not need to be the same or similar, but instead simply processed in such a manner that a comparison can be made. For example, the binding results and the MS results may vary by a factor of ten, one hundred or one thousand and still be straightforward to compare by scaling.

More preferably, the upscaling or downscaling is carried out so that the maximum binding signal value with respect to either a series of fractions or all fractions analysed in the binding array analysis is the same as (or corresponds to, as discussed above) the maximum relative abundance with respect to either a series of fractions or all fractions analysed (as appropriate) as determined by MS.

The extent of upscaling or of downscaling is generally carried out using a measure (be that mean, or median or maximum) that reflects the binding signal intensities and/or the abundance values with respect to a series of the fractions analysed.

By “series”, as used herein, it is understood that the mean, or median or maximum may be determined with respect to a subset of the fractions analysed. This is particularly relevant when separation has taken place on the basis of more than one parameter (for example more than one cell type) (as discussed above in the separation section), under which circumstances the series of fractions may relate to only one or only some of the fractions with respect to one or more parameters, but some or all of the fractions with respect to another parameter. By way of example, if fractionation was carried out with respect to subcellular location and size, a series of fractions may relate to one particular subcellular location, but some or all of the size ranges. Conversely, a series of fractions may relate to one particular size range but some or all of the subcellular locations.

A series of fractions as used herein may also refer to a set of neighbouring or consecutive fractions, e.g. when separation is based on size separation.

Thus, in an embodiment of the present invention, the processing of the binding results detected in step (ii) and the mass spectrometry results from step (iii) is carried out by either upscaling or downscaling the binding results (for example by converting to a percentage or proportion as described elsewhere herein) so they can be compared against the MS results, or conversely upscaling or downscaling the MS results (for example by converting to a percentage or proportion as described elsewhere herein) so that they can be compared against the binding results, wherein the upscaling or downscaling is carried out so that the maximum binding signal value with respect to either a series of fractions or all fractions analysed is the same as, or corresponds to, the maximum relative abundance with respect to either a series of fractions or all fractions analysed as determined by MS.

It is more preferable that the upscaling or downscaling (or the data processing that takes place before correlation) is generally carried out using a measure (be that mean, or median or maximum) that reflects the binding signal intensities and/or the abundance values with respect to either a series of fractions or all of the fractions analysed. This can be a powerful tool when applied to fractions on the basis of two or more parameters (for example two or more different samples e.g. samples from two or more different cell types). This is because, when only one parameter (e.g. a single cell type) is analysed and the maximum binding signal value is in the same fraction as the maximum relative abundance, the level of correlation is likely to be high as a result of the upscaling and/or downscaling process aligning the maximum binding signal value with the maximum relative abundance. However, when more than one parameter is analysed (for example the same polypeptide in a different cell type), then this allows a second dimension to be brought into the analysis, for example the relative abundance (differential protein expression) of the polypeptide in the two cell types. In this case, the fraction with the maximum binding intensity is less likely to be the same as the fraction for the maximum relative abundance, and as a result the heights of the binding signal intensities and the relative abundance are less likely to be the same after the upscaling and/or downscaling process. This in turn means that, when more than one parameter is analysed, high levels of correlation or overlap are less likely to occur and when they do occur, they are more likely to indicate that a binding agent is specific for the polypeptide of interest.

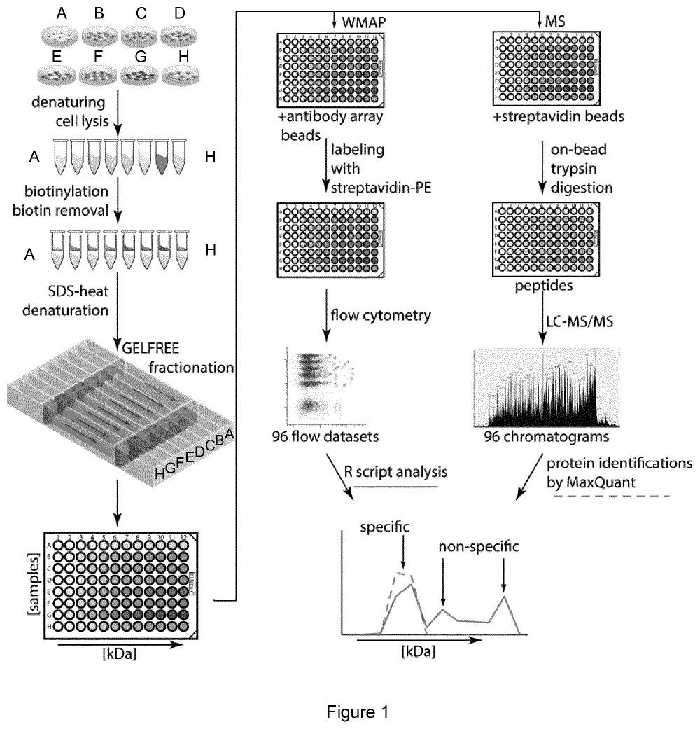

By way of example, the graph presented in FIG. 1 presented binding results (solid line) and MS results (dashed line) based on one cell type only (i.e. a single sample). In other words, fractionation has taken place with respect to one parameter only (size). In this setup, the main determinant regarding whether a high level of correlation (based here on the overlap of the peaks) is seen is based on the fraction in which the maximum binding signal intensity and the highest relative abundance (determined by MS) is seen. Thus, emphasis regarding the correlation analysis is placed on the fraction number (i.e. the x axis), and when the maximum binding signal intensity and the highest relative abundance are observed in the same fraction (as in FIG. 1), the likelihood that a high level of correlation or overlap will be seen may be relatively high and therefore there may be a risk of identifying binding agents thought to be specific to the polypeptide of interest but which are actually not because they bind to a different polypeptide found in the same fraction.

By using further additional parameters, such as varying cell type, a second parameter can be added in the form of relative heights of the peaks in the different cell type samples which in this case can reflect the abundance (relative abundance) of a polypeptide in the cell type. In this case the fraction with the maximum binding signal intensity is much less likely to be the same as the fraction with the highest relative abundance, and so the height of the signal (the y-axis) which can reflect the abundance of a polypeptide in the cell type, becomes more important in the correlation analysis. Thus, the height of the signal (e.g. the abundance or relative abundance of the polypeptide) acts as a second dimension of correlation analysis. As the likelihood of any two proteins being present in the same fraction and at the same abundance in two cell types is low, and will get lower the more cell types you analyse, this second dimension, e.g. relative abundance, may result in an improved or more precise assay. This, in turn, means that the likelihood of a false positive (i.e. a binding agent thought to be specific for the polypeptide when it is in fact not) is reduced.

In all the methods of the invention, the more samples that are analysed, the more complex is the data signature for a particular polypeptide (e.g. the relative abundance of a protein in 10 cell types is a more complex signature than relative abundance in two cell types). A more complex signature can result in an improved or more precise assay as it allows more certainty that the signature is indeed that of the intended target polypeptide.

In a further preferred embodiment, correlation step (iv) comprises the steps of:
a) determining the relative abundance of the polypeptide of interest within each fraction from the mass spectrometry results from step (iii);
b) plotting the binding signal intensity for a polypeptide binding to a specific binding agent detected in step (ii) against each fraction;
c) overlaying the relative abundance data determined in step a) with the binding results of step b); and
d) determining the level of overlap between the mass spectrometry results and the binding results;
or wherein step (iv) comprises the steps of:
a) determining the relative binding signal intensity for a polypeptide binding to a specific binding agent detected in step (ii) within each fraction;
b) plotting the abundance of the polypeptide of interest within each fraction from the mass spectrometry results from step (iii) against each fraction;
c) overlaying the relative binding signal intensity data determined in step a) with the abundance results of step b); and
d) determining the level of overlap between the mass spectrometry results and the binding results.

In such methods step a) can be carried out before, at the same time as, or after step b).

The term “plotting” relates to simply arranging the data so information with respect to each fraction (be that binding results/binding data, for example binding array results or data, or MS results/MS data) can be straightforwardly reviewed. As such, plotting includes tabulating the data as discussed above.

The extent of correlation between the binding results (binding agent array results/data) and MS results/data can be defined as a specificity index. A further indication as to the level of correlation can be measured as a percentage (or proportion) of the binding results (or binding agent array results) that overlaps with the MS results, for example as a percentage (or proportion) of the binding signal intensities from the binding results that overlaps with the abundance values from the MS results once any necessary data processing has been carried out so that a comparison can be made. The specificity index is also known as the correlation index or the overall correlation, for example the overall correlation between the signal values obtained with the binding/array analysis and MS.

Normalization can be a very effective tool for the correlating step. The process of normalization of data is well understood in the field of statistics and can be carried out by known and standard statistical techniques. Thus, a step of normalization of the data can be carried out before the step of correlating the binding results and the MS results and is a further example of how the data may be processed.

One very useful normalization technique in the methods of the present invention involves the data from individual fractions being normalized to the sum of a number (or series) of fractions. For example, the values from the binding results can be converted into percentage binding signal intensity values by dividing the binding signal intensity from each individual fraction by the total binding signal intensity across a series of fractions. It is also possible to determine percentage relative abundance through carrying out similar calculations with respect to the abundance values obtained from MS (dividing the abundance value from each individual fraction by the total abundance across a series fractions), and by doing so, the percentage binding signal intensities can be directly compared (with respect to correlation or overlap) with the percentage relative abundance values.

When analyses are carried out based on a series of size fractions (for example), a skilled person can determine the sum of the values in the peak in each cell type (for example) both for the MS results and for the binding results (binding array results), and then assess the correlation in the sums for different cell types. Thus, if the polypeptide of interest has the following relative abundance in cell types A>B>C>D>E we can calculate the sum of the signal in the peaks both for binding results (binding array results) and the MS results and determine the correlation between the two series of numbers obtained this way.

Another effective tool for data processing before correlation is a simple ranking of results. Such a step involves taking the numerical values for binding results from a set or series of fractions and ranking them based on numerical value, e.g. from lowest to highest or highest to lowest. A similar ranking procedure is carried out on the numerical values for the MS results from the same set or series of fractions which have been assessed in parallel. For example, if the values in one series is 1, 5, 1000, and the other is 10, 20, 30, the ranked series would both be 1, 2, 3. This means that there is a very good correlation between the binding and MS data, indicating that the binding agent in question is specific for the polypeptide of interest. Conversely, if the values in one series is 1, 5, 1000, and the other is 30, 10, 20, the first ranked series would be 1, 2, 3 and the second ranked series would be 3, 1, 2, which would indicate that there is no correlation between the binding and MS data, indicating that the binding agent in question is not specific for the polypeptide of interest. A statistical technique such as Spearmann-Rank correlation can be used.

In the methods of the invention, it is surprising that even though the binding results and the MS results are complex, these results are compatible, and the correlation step produces simple and readily interpretable data. Indeed, the correlation obtained between the binding results data (e.g. antibody data) and the MS results data is unexpectedly high.

The correlation step or the specificity index or the amount of overlap of the binding results and the MS results as described herein not only provides information regarding whether a binding agent is specific to a polypeptide in the mixture, but also provides information regarding whether a binding agent is specific for the polypeptide of interest, for example the polypeptide to which it is supposed to be binding. In particular, the binding results alone provide a high level confidence that a binding agent is specific for a polypeptide in the mixture (for example a single peak is observed), but a lower level of confidence that the binding agent is specific for the polypeptide of interest, for example the polypeptide to which it is supposed to be binding. The MS results provide information regarding the abundance at which a polypeptide of interest is present within each fraction but no information with respect to binding. The correlation between the binding results and the MS results therefore provides a level of confidence that the binding agent is specific and that the binding agent is specific for the polypeptide of interest.

Correlation is a statistical term (statistical correlation) which refers to a mutual relationship or connection between two or more things, and the step of correlating in the methods of the invention as described herein (e.g. step (iv)) thus refers to the process of establishing a relationship or connection between two or more things. As would be well understood by a person skilled in the art, assessing correlation is a statistical technique that can show whether and how strongly pairs of variables are related. The assessment of correlation generally requires the variables being analysed to be represented by meaningful numerical values and thus is readily applicable to the sets of numerical results/data generated by the methods of the invention in the form of the binding results, e.g. detected in step (ii) of the methods, and the MS results, e.g. from step (iii). The most common technique for measuring statistical correlation is the Pearson correlation and this is preferred for use in the methods of the present invention.

For validation (or assessment) of the specificity of an antibody (or other binding agent) based on correlation with results obtained with another method (in this case the correlation of binding results, e.g. detected in step (ii), and the mass spectrometry results, e.g. from step (iii)), the correlation should be statistically significant. Significance is measured as the likelihood that the correlation occurs by chance. It is common to operate with a probability of 5% or less that the correlation is random (i.e. p≤0.05). However, with the methods of the present invention, it has been established that higher % probabilities are also relevant. In this regard, the correlations as reported herein are Pearson correlations for linear data. Methods of calculating such correlations would be routine to a person skilled in the art. Exemplary methods of calculating these correlations are set out in the Examples. In particular, to assess the frequency of random correlations, the correlations between data in neighbouring rows (e.g. between mismatched data series, e.g. from protein A and protein B, e.g. from data within the MS data set) was assessed. The chance that results from two measurements correlate by chance is lower the more fractions you have.

Thus, in the methods of the present invention the significance of correlation is assessed and a correlation which is statistically significant is indicative of a binding agent that is specific for the polypeptide of interest. In preferred embodiments of the present invention, a correlation which is statistically significant with a probability of p≤0.20, p≤0.15, p≤0.10, or p≤0.05 is indicative of a binding agent that is specific for the polypeptide of interest. In the present methods, a probability of p≤0.05 is preferred.

A high specificity index (i.e. an index at or nearing 100%) is indicative of a binding agent that is specific for the polypeptide of interest. Preferably, the specificity index is above 80%, 82%, 84%, 86%, 88%, 90%, 91%, 92%, 93%, 94%, 95%, 96%, 97%, 98% or 99% (or the equivalent proportion). Most preferably, the specificity index is 100% (or 1.0 as a proportion), which means that there is complete overlap between the binding results and the MS results.

Thus, a level of overlap of more than 80%, preferably 85%, more preferably 90%, is indicative of a binding agent that is specific for the polypeptide of interest. Thus, an overall correlation or specificity index of more than 0.80, 0.85 or 0.90 is also indicative of a binding agent that is specific for the polypeptide of interest, although in some circumstances a correlation threshold of 0.70 (70%) will be sufficient.

In a preferred embodiment, indexes in addition to the specificity index are used in order to provide further information about the binding agent being analysed, for example further confidence that a binding agent is specific for a polypeptide of interest. These indexes may include a core index, a wide (or width) index, a signal index and an absolute signal intensity.

In order to determine the core and wide indexes, one must first determine the MS centre. This is the fraction with the highest relative abundance (e.g. the fraction with the highest signal intensity) or abundance of the polypeptide of interest obtained from the MS data in relation to a series of fractions or in relation to all the fractions. For example with respect to FIG. 2A the MS centre is fraction 10, as fraction 10 shows the highest relative abundance (highest signal intensity) as shown by the dashed lines.

The core index (peak position) is the sum of the binding signal intensity from the binding agent array analysis (array signal) measured in the fraction corresponding to the MS centre and the two immediate neighbouring fractions (i.e. in three fractions total) divided by the sum of the binding signal intensity measured in either a larger series of fractions or all fractions (total signal). The neighbours are always immediately either side of the MS centre, i.e. one on each side of MS centre. For example with respect to FIG. 2A the core index is the sum of the binding signal intensity (solid lines) measured in fractions 9 to 11 divided by the sum of the binding signal intensity measured in all twelve fractions/fractions 1 to 12 (total signal). Thus, in this example, the core index is calculated from the results of three fractions out of a total of 12 fractions, i.e. 25%. If the total number of fractions is different then the number of neighbouring fractions to be used to calculate the core index can be adjusted accordingly.

The higher the core index (i.e. the closer the core index is to 1), the more specific the binding agent for the polypeptide of interest. Preferably, the core index is above 0.70, 0.72, 0.74, 0.76, 0.78, 0.80, 0.82, 0.84, 0.86, 0.88, 0.90, 0.91, 0.92, 0.93, 0.94, 0.95, 0.96, 0.97, 0.98 or 0.99. More preferably, the core index is 1.0.

An alternate measure of the core index (peak position) is to assess whether the maximum binding signal intensity from the binding agent array analysis (array signal, e.g. maximum antibody signal) occurs in the same fraction as the maximum MS signal for the same cell type, or in one of the immediate neighbouring fractions (i.e. MS centre +/−1). If yes, then the binding agent passes this criteria. If no, then the binding agent fails this criteria.

The wide index (otherwise known as the width index) is similar to the core index but different in that the number of fractions compared against either a larger series of fractions or all of the fractions is larger. In particular, the wide index (which can be regarded as a proxy for relative protein abundance) is the sum of the binding signal intensity from the binding agent array analysis (array signal) measured in the fraction corresponding to the MS centre and the two immediate neighbouring fractions on each side of the MS centre (i.e. in five fractions total) divided by the sum of the binding signal intensity measured in either a larger series of fractions or all fractions (total signal). For example with respect to FIG. 2A the wide index is the sum of the binding signal intensity (solid lines) measured in fractions 8 to 12 divided by the sum of the binding signal intensity measured in all twelve fractions/fractions 1 to 12 (total signal). Thus, in this example, the wide index is calculated from the results of five fractions out of a total of 12 fractions, i.e. approximately 40 to 45%. If the total number of fractions is different then the number of neighbouring fractions to be used to calculate the wide index can be adjusted accordingly.

The higher the wide index (i.e. the closer the wide index is to 1), the more specific the binding agent for the polypeptide of interest. Preferably, the wide index is above 0.70, 0.72, 0.74, 0.76, 0.78, 0.80, 0.82, 0.84, 0.86, 0.88, 0.90, 0.91, 0.92, 0.93, 0.94, 0.95, 0.96, 0.97, 0.98 or 0.99. More preferably, the wide index is 1.0.

Core and wide indexes can generally be determined only when, during separating the polypeptides in the mixture into a plurality of fractions (step (i) of the method), one or more series of continuous fractions are formed. “Continuous” means that, when the fractions are plotted along an axis (e.g. the x axis), they can be arranged with respect to a scale that is either increasing or decreasing. The scale may be linear or logarithmic, but it is often linear. Once the data are arranged, neighbours of a particular data plot (or data point) are then somehow related to the data plot (for example, with respect to size fractions, the neighbours of a data plot would be the next smallest or largest fractions in comparison to that data plot). Examples of separating that may form one or more series of continuous fractions include separating on the basis of a physical parameter such as differential mass, acidity, basicity, charge, hydrophobicity or affinity towards a ligand of interest. Examples of separating that, alone, would likely not form one or more series of continuous fractions include methods for crude separation of proteins into major subcellular compartments such as cytosol, membranes and nuclei.

Core and wide indexes can generally be determined only when the MS centre is sufficiently far removed from the smallest value fraction (in terms of fraction number) and largest value fraction (in terms of fraction number). In particular, neither core nor wide indexes can generally be determined when the MS centre is the smallest value fraction or the largest value fraction. Furthermore, the wide index cannot generally be determined when the MS centre is the second smallest value fraction or the second largest value fraction. For example, with respect to FIG. 2A, an example with twelve fractions, a core index can be determined only when the MS centre is one of fractions 2 to 11 and a wide index can be determined only when the MS centre is one of fractions 3 to 10. If a skilled person is not able to determine core indexes or core and wide indexes for these reasons, he may repeat the claimed method with smaller or larger value fractions (depending on whether the MS centre is too low or too high) so that the indexes may be determined, or select a different fractionation method to obtain a higher resolution. By way of example, if fractionation was carried out by gel electrophoresis with a 5% gel and the polypeptide of interest was found to be in the fraction with the smallest polypeptides (fraction 1), the skilled person may repeat the fractionation process with a higher concentration gel, such as an 8% gel, in order to further fractionate the smaller polypeptides present in the mixture and move the polypeptide of interest into a higher fraction.

It is generally important that the core and the wide indexes use the MS relative abundance or abundance results (e.g. the fraction with the highest signal intensity) in order to set the MS centre and thus determine which fractions are compared against which, but then compare the binding signal intensity results (binding agent array results) in these fractions, i.e. compare the MS data with the binding array data, as this cross-comparison can provide an indication not only that the binding agent being analysed is specific, but more importantly that the binding agent is specific for the polypeptide of interest (i.e. the intended binding agent target, e.g. antibody target) as determined through the cross-reference to the MS data.

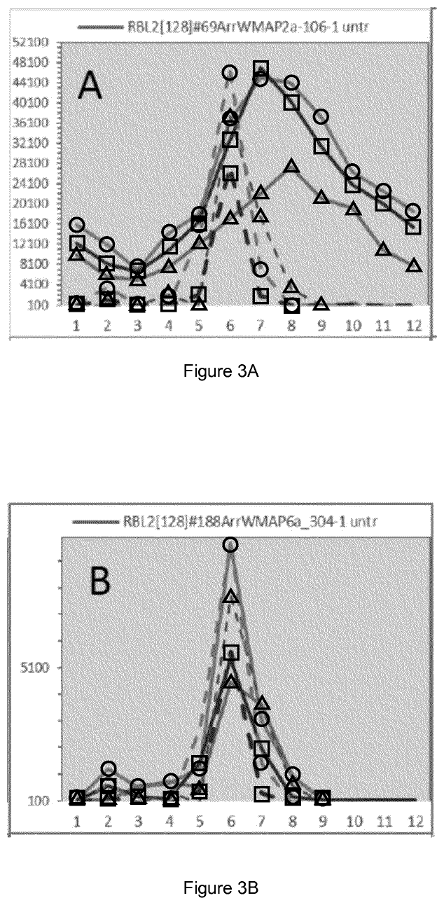

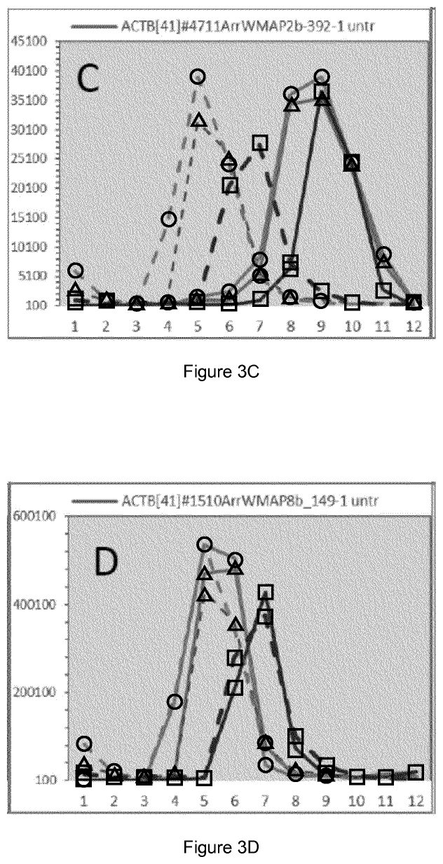

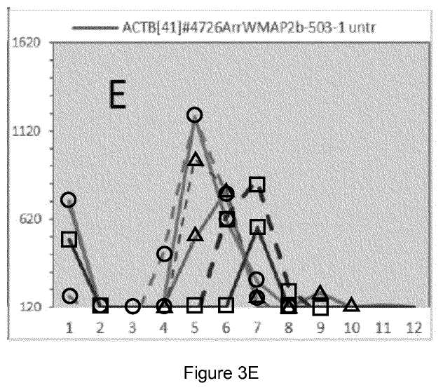

A binding agent that is specific but for a polypeptide (or other entity) other than the polypeptide of interest would likely have low wide and/or core indexes because the MS centre would be set at a different point, and possibly a significantly different point, than the fraction number with the highest binding signal intensity results (binding agent array results). As a purely illustrative example, the MS centre might be set at fraction 3, but the signal peak (binding signal intensity peak) might be at, for example, fraction 9, and so most of the total signal intensity would fall outside of fractions 2 to 4 (with respect to the core index) and outside of fractions 1 to 5 (with respect to the wide index). Such an analysis can be used to identify cross reactive or non-specific antibodies, i.e. antibodies which bind with other entities (for example other polypeptides) than the polypeptide of interest. An example of the identification of such cross reactive or non-specific antibodies is shown in FIG. 3A and FIG. 3C.

It will be appreciated that a minimum number of four fractions is necessary in order to determine the core index (three fractions that form the core region and at least one additional fraction to compare against). Similarly, the minimum number of fractions necessary in order to determine the wide index is six fractions (five fractions that form the wide region and at least one additional fraction to compare against).

Given in practice the wide nature of the binding analysis data, it is more preferable to use the wide index over the core index. However, it is even more preferable that both the wide and core indexes are calculated.

It will be appreciated that variations in the width of the “core” or the “wide” regions (as shown in FIG. 2A) can be varied depending on the abundance of the polypeptide in a mixture. For example, actin is a polypeptide that is abundant is some cell types, and so analysing binding agents specific for such an abundant polypeptide may result in wide MS and binding peaks. Under such circumstances, the core and/or the wide area can be widened accordingly. For example, the wide region, rather than covering five fractions as described above, may cover seven, nine, eleven, thirteen, fifteen, seventeen or nineteen fractions. It will be understood that if the wide region needs to increase then the number of fractions that form the series may need to increase also (for example, the series would need to comprise at least eight fractions when the wide region is seven, the series would need to comprise at least ten fractions when the wide region is nine, the series would need to comprise at least twelve fractions when the wide region is eleven, the series would need to comprise at least fourteen fractions when the wide region is thirteen, the series would need to comprise at least sixteen fractions when the wide region is fifteen, the series would need to comprise at least eighteen fractions when the wide region is seventeen and the series would need to comprise at least twenty fractions when the wide region is nineteen).

A comparison between the wide and the core indexes can also provide an indication of whether a binding agent is specific for the polypeptide of interest, as a wide index that is the same as a core index indicates that all of the signal that fall within the wide region falls with the core region also. Thus, it is preferred that the difference between the core and the wide indexes is less than 0.1, 0.09, 0.08, 0.07, 0.06, 0.05, 0.04, 0.03, 0.02 or 0.01.

The signal index (or signal to noise ratio) corresponds to the maximal binding signal intensity from the binding agent array analysis (array signal), taken from either a series of fraction or all analysed fractions, divided by the median binding signal intensity. Maximal and median binding signal intensities are shown in FIG. 2A. The higher the signal index, the greater the binding sensitivity of the binding agent. Preferably the signal index is not less than 3, 4, 5, 6, 7, 8, 9, 10, 12, 14, 16, 18 or 20 (or is more than or at least 3, 4, 5, 6, 7, 8, 9, 10, 12, 14, 16, 18 or 20). Preferably the signal index is between 4 and 50, more preferably between 5 and 40, more preferably between 6 and 30. Signal intensity or signal values can be measured by any appropriate means. For example, when flow cytometry is used to obtain the binding array data then a convenient measure would be median fluorescence intensity (MFI).

The absolute signal intensity (otherwise known as the maximum fluorescence intensity or the maximal signal intensity) is simply the maximal binding signal intensity from the binding agent array analysis (array signal) measured for a particular binding agent. In general, the higher the absolute signal intensity, the greater the binding sensitivity of the binding agent. Again, signal intensity or signal values can be measured by any appropriate means. For example, when flow cytometry is used to obtain the binding array data then a convenient measure would be median fluorescence intensity (MFI). Preferably the absolute signal intensity is above 1500, 2000, 2500, 3000, 3500, 4000, 4500, 5000, 6000, 7000, 8000, 9000 or 10000. Preferably the absolute signal intensity is between 1500 and 100000, more preferably between 2500 and 80000, more preferably between 3500 and 60000, more preferably between 5000 and 40000.

In a preferred embodiment, a computer algorithm is used in order to carry out the correlation step or to determine at least the level of correlation. Preferably a computer algorithm is used to carry out the upscaling and/or downscaling discussed above, and from this the level of correlation or the level of overlap can be determined. More preferably a computer algorithm is used to determine the specificity index, more preferably the specificity index and one or more additional indexes, e.g. indexes as described above, more preferably all of the indexes described above. The computer algorithm may be developed using methods and programs readily available to the skilled person. By way of example, in the case of the present invention a Microsoft Excel® function can be used to identify the fraction with the highest signal intensity and the fraction with the highest abundance (the MS centre), and regions can be set around the centre to include the nearest of the two or four nearest neighbouring fractions (for the purposes of determining the core and wide indexes discussed above). In this scenario, a simple Excel spread sheet can for example be used to assess the proportion of the signal intensity from binding agent array analysis that is found in the centre and the immediate neighbouring fractions (i.e. centre +/−1 fraction, or centre +/−2 fractions). Additional parameters include correlation between the MS and binding agent-derived signals measured across the fractions (correlation) and absolute signal intensity (threshold). Other programs suitable for creating these algorithms (in addition to Excel) include Rstudio® R-, SPSS® and MathLab® and many others well known to a person skilled in the art.

The indexes are useful for setting thresholds (or criteria or validation criteria), which can for example be highly useful in antibody validation. Such thresholds can be used for screening data relating to numerous binding agents in order to quickly decide which binding agents are specific and/or sensitive. The minimum specificity index, wide index and/or core index would form thresholds that relate to minimum level of specificity expected from the binding agent. The minimum signal index and/or absolute signal intensity can form a threshold that relates to the minimum level of sensitivity expected from the binding agent, but the minimum signal index and/or absolute signal intensity can also form a threshold that relates to the signal level above which is considered to be a true signal (i.e. indicative of a binding agent binding to a polypeptide) rather than noise.

Preferable screening thresholds include the combination of one or more of (i) specificity index, (ii) a signal index (iii) absolute signal intensity, and, optionally (iv) a core index.

Preferable screening thresholds include the combination of one or more of (i) a specificity index of 80% or above, (ii) a signal index of above 3, preferably above 4, (iii) an absolute signal intensity of greater than 5000 (after subtraction of background) and, optionally (iv) a core index of greater than 0.7.

Other screening thresholds include the combination of (i) a signal index of at least 4 and (ii) a positive (or pass for) peak position (i.e. the maximum binding signal intensity from the binding agent array analysis (array signal, e.g. maximum antibody signal) occurs in the same fraction as the maximum MS signal for the same cell type or in one of the immediate neighbouring fractions (i.e. MS centre +/−1)).

Alternative screening thresholds include the combination of one or more of (i) correlation (specificity index), wide index, absolute signal intensity (after subtraction of background) and signal index. If all of these are used then an overlap index of at least 0.6 or preferably 0.8 can also be used to validate binding agent specificity.

Although it is preferable to determine binding agents that are specific for a particular polypeptide of interest using the methods of the present invention, it will be appreciated that such methods can be used to determine cross-reactive or non-specific binding agents also. As shown in FIGS. 1, 2C, 3A and 3C, cross-reactivity (or non-specific binding) may be present when a measurable or high binding signal (or peak) is seen in one or more fractions that do not have corresponding high abundance as determined by mass spectrometry (or in other words that do not overlap or match with the MS data or MS peak). In a preferred embodiment, information regarding the polypeptide that the binding agent is cross-reactive with may be obtained by appropriate analytical techniques, for example, by carrying out further mass spectrometry analysis as discussed below.

### Downstream Analysis

The method of the present invention as described in steps (i) to (iv) can be used as a starting point for further analysis with respect to either a polypeptide of interest and/or a binding agent for a polypeptide of interest.

For example, in a preferred embodiment the method of the present invention further comprises the steps of:
(v) determining one or more fractions which are enriched for a particular polypeptide of interest;
(vi) contacting the one or more fractions with a binding agent to said polypeptide of interest attached to one or more solid supports;
(vii) disrupting the binding agents of step (vi) from the associated polypeptides; and
(viii) contacting the released polypeptides with a plurality of binding agents attached to one or more solid supports and detecting the binding of the polypeptides to the binding agents.

Such a method may be used to determine if one or more of the binding agents used in steps (viii) bind to the same polypeptide as the binding agent used in step (vi). Such a method may also be used to analyse whether or not polypeptide complexes, rather than individual polypeptides, have bound to the specific binding agent of step (vi), as disruption step (vii) would also disrupt at least some of these complexes. Contacting step (viii) could then be used to detect not only polypeptide complexes but also the individual polypeptides making up the complexes. A schematic illustrating an example of these additional steps is shown in FIG. 5.

The plurality of binding agents used in step (viii) can be any plurality of binding agents as described elsewhere herein in which a number of different binding agents are present. Thus, any appropriate array or library of binding agents can be used, for example the array (plurality of binding agents) of step (ii) could be used or an alternative array. The array may or may not include the binding agent of step vi). Appropriate solid supports are also described elsewhere herein as well as appropriate methods of detection (for example the use of labelled polypeptides and particles with different detectable features).

Alternatively, in a further preferred embodiment the method of the present invention further comprises the steps of:
(v) determining one or more fractions which are enriched for a particular polypeptide of interest;
(vi) contacting the one or more fractions with a binding agent to the polypeptide of interest attached to one or more solid supports;
(vii) disrupting the binding agents of step (vi) from the associated polypeptides;
(viii) contacting the released polypeptides with a soluble binding agent that binds specifically to a first epitope on the polypeptide of interest; and
(ix) contacting the polypeptides bound to said soluble binding agent with a plurality of binding agents attached to one or more solid supports and detecting the binding of the binding agents attached to the one or more solid supports to the polypeptides of interest.

Step (ix) thus allows binding agents (for example antibodies) which bind to different epitopes, for example second or third, etc., epitopes (i.e. not the first epitope) on the polypeptide of interest to be identified, as binding agents which bind to the same epitope as the soluble binding agent used in step (viii) will be blocked or prevented from binding by the soluble binding agent. Such a method may thus be used as an epitope binning tool that allows one to identify two (or more) binding agents that bind to different epitopes of a polypeptide of interest. As discussed in the background art section, such binding agent pairs are highly sought after as the use of such pairs provides a very high level of specificity in ELISA, proximity ligation and immunoprecipitation WB assays. Such pairs of binding agents can be very hard to identify using conventional techniques and the high throughput advantage of this method means that such binding pairs can be found more straightforwardly.

As described above, polypeptide complexes rather than individual polypeptides may have bound to the binding agent in this epitope binning context also. For this reason, although generally it is preferable that the different binding agents attached to one or more solid supports as described in step (ix) are specific for the polypeptide of interest, this step may be carried out with binding agents specific for a variety of polypeptides in order to analyse the nature of any polypeptide complexes that may have formed.

The soluble binding agent or binding agent as described in steps (vi) (viii) and/or (ix) may not be a binding agent specifically directed to a polypeptide of interest, e.g. a single polypeptide of interest, but may instead be a binding agent with a more generic binding profile that can bind to many polypeptides. For example, the binding agent may be a general motif-specific binder (e.g. a motif specific antibody), e.g. that binds to phosphorylated amino acid residues such as phosphorylated tyrosine residues or another post-translational modification. Such binding agents may be antibodies or other types of binding agent as described elsewhere herein, including chemicals or small molecules. For example, the binding agent may be phenylphosphate, a small molecule capable of blocking all epitopes containing phosphorylated amino acids (i.e. prevent binding agents specific for epitopes containing phosphorylated amino acids from binding). Another example is to use an anti-phosphotyrosine antibody as the soluble binding agent in order to block the binding of binding agents specific for phosphorylated tyrosine epitopes.

Phenylphosphate or anti-phosphotyrosine antibody can thus be used to bind to phosphorylated residues in the polypeptide of interest meaning that binding agents capable of binding to non-phosphorylated epitopes can be identified. They can also be used to determine whether or not a polypeptide of interest is phosphorylated.

In this method, it is generally important that step (viii) is carried out before step (ix) so that in step (ix), only binding agents specific for epitopes other than the epitope occupied by the soluble binding agent of step (viii) will be found. In such methods the soluble binding agent of step (viii) will comprise multiple copies of the same binding agent in order to ensure that all (or substantially all) of the epitopes on the polypeptide of interest recognised by the soluble binding agents are bound.

The plurality of binding agents used in step (ix) can be any plurality of binding agents as described elsewhere herein in which a number of different binding agents are present. Thus, any appropriate array or library of binding agents can be used. By using such an array or library it would be possible to assess polypeptides that have formed a complex with the polypeptide of interest, as discussed above. In some embodiments it is preferable that the different binding agents are directed to the polypeptide of interest, i.e. the polypeptide targeted by the soluble binding agent. However, other binding agents, including more general binding agents, may be used as outlined above, such as motif-specific binding agents (e.g. antibodies) or binding agents (e.g. antibodies) to associated polypeptides (e.g. polypeptides that have formed a complex with the polypeptide of interest) or binding agents (e.g. antibodies) that are potentially cross-reactive with the protein of interest). Appropriate solid supports are also described elsewhere herein as well as appropriate methods of detection (for example the use of labelled polypeptides and particles with different detectable features).

Alternatively, in a further preferred embodiment the method of the present invention further comprises comprising the steps of:
(v) determining one or more fractions which are enriched for a particular polypeptide of interest;
(vi) contacting the one or more fractions with a binding agent to the polypeptide of interest attached to one or more solid supports;
(vii) disrupting the binding agents of step (vi) from the associated polypeptides; and
(viii) assessing the amino acid composition of the released polypeptides by mass spectrometry.

Such additional method steps may be carried out in order to provide further confidence that a binding agent identified as specific by carrying out steps (i) to (iv) of the method is specific for the polypeptide of interest and not for another polypeptide that, by coincidence, is present in a high abundance in the same fraction as the polypeptide of interest. However, as discussed above, the method as described in steps (i) to (iv) is likely to identify a binding agent that is specific for the polypeptide of interest (assuming that a correlation is indeed observed in step (iv)), and this additional MS step provides a way of verifying this. One advantage of using MS in step (viii) is that all the eluted/released polypeptides from the disrupting step (vii) (which can conveniently be carried out as part of the digestion of polypeptides in preparation for the MS step) can be detected. In contrast with the methods described above which use binding agent (e.g. antibody) arrays for this step, the use of MS means that polypeptides present in the samples can be identified even if there is not a binding agent/antibody to that protein present in the array. Appropriate methods for carrying out the MS assessment of step (viii) would be well known to a person skilled in the art and are described elsewhere herein.

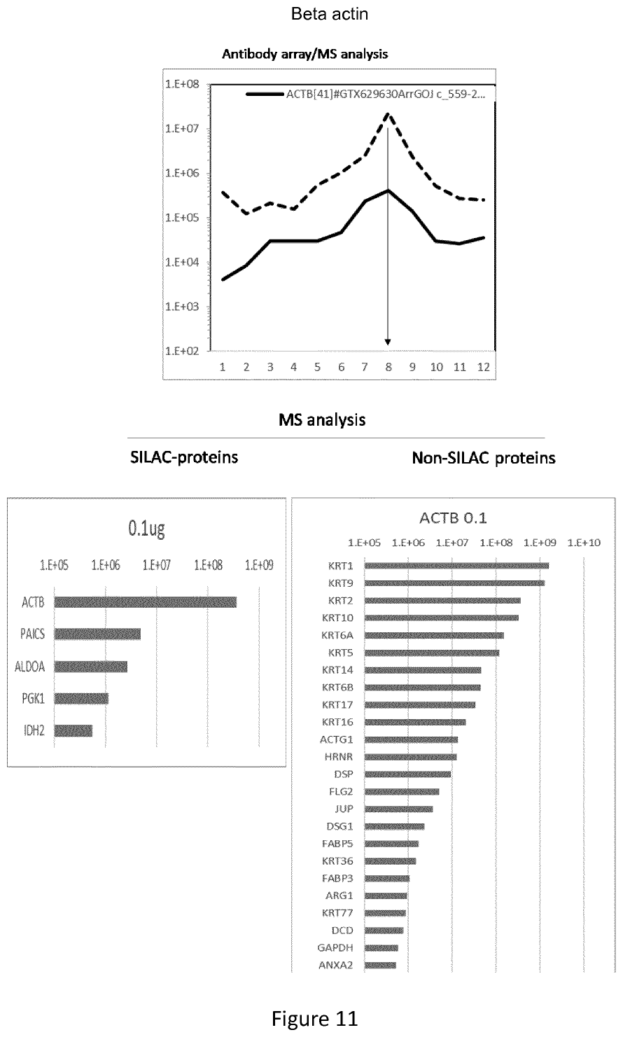

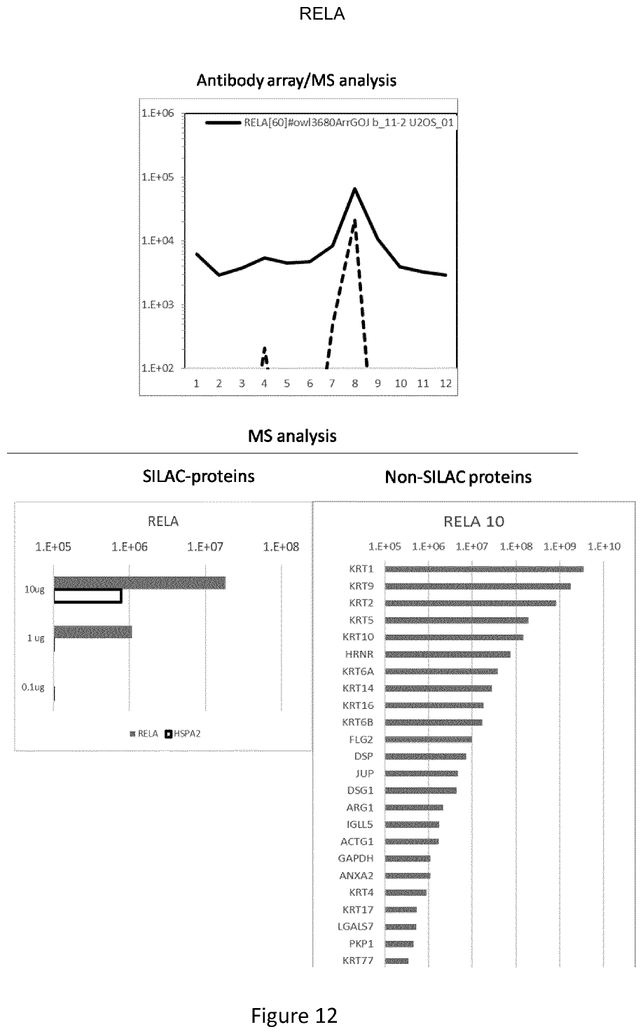

Preferably step (vi) is an IP (immunoprecipitation) step and step (viii) is an MS step. Thus, steps (vi) to (viii) together describe a process of IP-MS. IP-MS techniques are known in the art and when carried out with a single antibody on a total native cell sample/lysate generally contain several hundred proteins, making analysis of which of these proteins binds directly to the antibody impossible. Such standard methods of IP-MS are therefore not useful to assess antibody specificity. Surprisingly the methods of the present invention in which IP-MS is carried out on an enriched fraction prepared using the fractionation and array analysis as described herein (i.e. steps (i) to (v) of the method of this embodiment) show extremely high purity. This is illustrated in FIGS. 11 and 12 where it can be seen that in contrast to prior art IP-MS methods (which contain several hundred proteins), the MS analysis step of the method of this embodiment (step (viii) above) gives rise to the detection of only a few proteins.

This is particularly the case when stable isotope labelling with amino acids in culture (SILAC labelling) is used. Thus, this embodiment can also preferably and advantageously be combined with a step in which the cells from which the polypeptides are derived for analysis are subjected to metabolic labelling with isotopes (e.g. SILAC labelling) as described elsewhere herein before step (i) is carried out. In other words, SILAC labelling of cells is carried out prior to step (i) of the method. Such metabolic labelling of polypeptides means that only sample (e.g. cell) polypeptides are labelled. As can be seen from the Examples and FIGS. 11 and 12, the MS analysis of step (viii) can be used to confirm specific binding of a binding agent (e.g. antibody) of interest to its target polypeptide. Surprisingly and advantageously this SILAC labelling has been shown to result in much less complex results from the IP (binding agent)-MS step (5 proteins or less as compared to at least 200 proteins for standard IP-MS). This is believed to be due at least in part to the fact that contaminating polypeptides are not labelled.

It is also shown that a surprisingly low amount of protein can be taken from the enriched fraction of step (v) and successfully used in the IP-MS steps (vi) to (viii). In this regard, as little as 10 μg, 1 μg or even 0.1 μg (100 ng) protein has been successfully used (see FIGS. 11 and 12). Thus, it can be seen that the methods of the invention are highly and surprisingly sensitive.

In the methods of the invention involving IP-MS, a preferred embodiment is one wherein the MS analysis is multiplexed using addressable bar codes (i.e. barcodes that are traceable to a single capture reaction, e.g. identifying a single binding agent or antibody). Any addressable bar code can be used, examples of which would be well known to a person skilled in the art. Preferably the addressable bar code is a stable isotope (e.g. the use of different SILAC labels or other isotope labels). Alternatively, the addressable bar code can be a physical parameter (for example protein size) specific for proteins in a certain fraction. In this embodiment for example if fraction 1 contains proteins smaller than 20 kDa and fraction 2 contains proteins larger than 40 kDa, then it is clear that any protein smaller than 20 kDa came from fraction 1 while those that are larger than 40 kDa came from fraction 2.

### Step (v)

The determination of the one or more fractions which are enriched for a polypeptide of interest in step (v) of all the above methods may be determined from the binding results determined in step (ii) of the method (for example from the binding agent array analysis results), for example by identifying the one or more fractions with the highest signal intensity, for example absolute signal intensity, with respect to binding agents that are considered specific for the polypeptide of interest. This means that one or more fractions may be determined without reviewing the MS results). Preferably the one or more fractions are determined by additionally reviewing or cross-checking with the MS results to determine those fractions which contain target polypeptides as verified by MS analysis. In other words, in a preferred embodiment, the enriched polypeptide was a polypeptide identified (e.g. as an antibody or binding agent target) by mass spectrometry in the previous steps of the method. Thus, preferred fractions are determined through the correlation step described in step (iv), for example are those which show good correlation, good overlap, high overall correlation or specificity index as discussed elsewhere herein between the binding results of step (ii) and the MS results of step (iii). Although one or more fractions can be used in such methods, the use of one fraction is preferred in some embodiments, for example if a single fraction is suitably enriched for the polypeptide of interest.

### Step (vi)

Once the fraction(s) are identified, step (vi) of all the above methods is conveniently carried out on a further aliquot taken from the fraction(s) of interest formed after separation step (i), for example by returning to the master plate. The binding agent of step (vi) may be a binding agent that is known in the art to bind specifically to the polypeptide of interest, or alternatively the binding agent may be one that has been determined to be specific for or to bind to the polypeptide of interest through using the method of the present invention as described in steps (i) to (iv). Thus, the binding agent need not be specific to the polypeptide of interest but could for example be a more general binding agent as described elsewhere herein. For example, a motif-specific binding agent (e.g. antibody) such as a binding agent to a phosphorylated amino acid or another post-translational modification, or binding agents (e.g. antibodies) to associated polypeptides (e.g. polypeptides that have formed a complex with the polypeptide of interest), or binding agents (e.g. antibodies) that are potentially cross-reactive with the protein of interest). It would therefore then be appreciated that the binding agent of step (vi) may or may not be a binding agent present in contacting step (ii) of the present invention. The binding agent of step (vi) is generally a single type of binding agent or a single specificity binding agent (for example one particular antibody) that is specific for the polypeptide of interest. Where antibodies are used this step can also be referred to as an immunoprecipitation (IP) step. Such IP steps are preferred in the methods of the present invention. Such IP steps (or equivalent steps using other types of binding agent attached to a solid support) can optionally be followed by dividing the sample into bound and unbound fractions and analysis (e.g. MS and/or binding array analysis) of the bound and/or unbound fractions as described elsewhere herein.

The nature of the solid supports that these binding agents are attached to are described in detail above.

Step (vii)

The disruption of step (vii) of all the above methods may be carried out using techniques readily known in the art for disrupting interactions between binding agents (for example antibodies) and associated polypeptides. For example, such techniques may involve exposing the binding agents to acidic conditions or through incubating the binding agents in an anionic surfactant such as sodium dodecyl sulphate. Alternatively, where such a disruption step is followed by an MS step, the digestion steps, e.g. trypsin digestion steps, that are required to prepare the polypeptides for MS analysis, provide a convenient means for carrying out the disrupting step.

However, the inventors have surprisingly found that disruption (sometimes referred to herein as elution) can be carried out using techniques previously thought to not be stringent enough to lead to disruption, as discussed in Example 2 and shown in FIG. 5. Thus, in a preferred embodiment, the disruption of step (vii) is carried out using conditions which are mild enough so as not to affect or disrupt the conformation of the polypeptides (or at least most polypeptides), in other words conditions or solutions which do not cause denaturation and/or unfolding of polypeptides (or at least most polypeptides).

Any suitable buffer may be used. However, in a preferred embodiment, the disruption of step (vii) is carried out using a phosphate buffered saline (PBS), a 4-(2-hydroxyethyl)-1-piperazineethanesulfonic acid (HEPES) buffered saline or a solution of phenyl phosphate (at a concentration of preferably 30 mM) either in the presence or absence of a non-ionic surfactant. The nonionic surfactant may be any suitable nonionic surfactant, including a polysorbate-type non-ionic surfactant, more preferably polysorbate 20 (Tween® 20). Alternatively, the non-ionic surfactant may include a maltoside surfactant, preferably dodecyl-maltoside. The skilled person can determine the suitable detergent/surfactant to use through routine experimentation (as discussed in more detail below), and the suitable salts to use also.

In these embodiments, a skilled person can determine an appropriate concentration of reagent to use in order to disrupt the interaction of the binding agents with their bound polypeptides but to preferably not affect the conformation of the polypeptides (i.e. to retain the conformation). By way of example, in a further preferred embodiment, the concentration of nonionic surfactant used is between 0.1% to 10%, preferably between 0.5% and 5%, more preferably between 0.8% and 1.2%.

In these embodiments, a skilled person can determine an appropriate temperature to use in order to disrupt the interaction of the binding agents with their bound polypeptides but to preferably not affect the conformation of the polypeptides. By way of example, in a preferred embodiment, the disruption of step (vii) is carried out without significant heating or at a temperature around room temperature, for example at a temperature of between 4° C. and 37° C., preferably 15° C. and 30° C., more preferably between 18° C. to 27° C. A temperature of between 21° C. and 23° C. is particularly preferred.

Again, in these embodiments, a skilled person can readily determine an appropriate time period to use in order to disrupt the interaction of the binding agents with their bound polypeptides but to preferably not affect the conformation of the polypeptides. By way of example, the disruption of step (vii) can be carried out for between five minutes to 24 hours, preferably between ten minutes and 12 hours, more preferably between twenty minutes and 6 hours, more preferably between twenty minutes and an hour. Alternative time periods might be between five to sixty minutes, ten to fifty minutes, or twenty to forty minutes. Preferably the disruption of step (vii) is carried out under constant agitation. The pH of the solution used is preferably between 6 and 8, more preferably between 6.5 to 7.5. The conditions such as those described above are considered to be mild disruption conditions that were previously not considered sufficient to disrupt the association (binding) between a binding agent with a binding affinity typical for antibodies and a polypeptide.

The advantage of using such mild disruption conditions is that the conformation of the disrupted polypeptide is not affected or is less likely to be affected, as shown in FIGS. 5B and 5C. Thus, such disruption conditions may be used in order to assess the binding of binding agents to conformation-specific epitopes as the conformation is retained. It is therefore preferable to use such disruption conditions with respect to fractions that were separated in step (i) using methods that do not affect the conformation of the polypeptides, such as size-exclusion chromatography.

These mild disruption conditions can also be used in order to determine whether or not a particular epitope is conformation-specific or not. Methods using these mild disruption conditions can also be used to identify binding agents (for example antibodies) which recognise conformation-dependent epitopes (see FIG. 5). Binding agents (e.g. antibodies) which recognise conformation-dependent epitopes can be particularly useful for IHC or IF.

In a preferred embodiment, the mild disruption discussed above is carried out using a polysorbate-type non-ionic surfactant at a concentration of between 0.5% and 5% at a temperature around room temperature (for example between 21° C. and 23° C.) and at a pH of between 6 and 8.

Further in this regard, in a further embodiment of the present invention, in the disruption step (vii), the binding agents are disrupted from the associated polypeptides using successive solutions with increasing stringency, for example a step using mild disruption conditions followed by a step with harsh disruption conditions (or harsher disruption conditions) to remove additional polypeptides.

A skilled person, would readily know which conditions would be considered stringent, or more stringent (or more harsh) than the mild conditions discussed above (for example the conditions currently used in the art to detach polypeptides from binding agents such as antibodies) and which conditions would be considered mild or less stringent (for example conditions previously thought to not be sufficient to lead to detachment, such as conditions generally used to wash the non-specific attachment of polypeptides to particles or the mild conditions described above).

The skilled person would also readily know how to test whether disruption conditions are sufficient for disruption but also able to maintain conformation-specific epitopes through determining a binding agent that is specific for a conformation-specific epitope (using, for example, the method of the present invention as described in steps (i) to (iv)), carrying out step (v) to (vii) above in order to bind a binding agent specific for the conformation-specific epitope to the polypeptide of interest and then disrupt this binding using conditions believed to maintain the conformation-specific epitope, then contacting the released polypeptide with a binding agent specific for the epitope and detecting the binding using the methods described above.

For example, in a preferred embodiment the first disruption conditions may be one of the mild conditions described above and the second disruption conditions may be any more harsh or more stringent (or harsh) condition such that additional polypeptides are disrupted from associated polypeptides. Preferably, the second disruption is carried out using an anionic surfactant, preferably an organosulphate surfactant, more preferably sodium dodecyl sulphate. In these embodiments, a skilled person can readily determine an appropriate concentration of reagent to use in order to disrupt the interaction of the binding agents with their bound polypeptides. By way of example, in a preferred embodiment, the concentration of anionic surfactant used is between 0.01% to 1%, preferably between 0.05% and 0.5%, more preferably between 0.08% and 0.12%.

Again, in these embodiments, a skilled person can readily determine an appropriate temperature to use in order to disrupt the interaction of the binding agents with their bound polypeptides. By way of example, in a preferred embodiment, the second disruption is carried out by heating, for example at a temperature of between 75° C. and 115° C., preferably between 85° C. to 105° C., more preferably between 90° C. and 100° C.

In an alternative embodiment, the second disruption conditions is an exposure to an acidic pH, such as a pH between 1 and 4, preferably between 1.5 and 3.5, more preferably between 2 and 3. This form of disruption would be carried out at temperatures similar to those described for mild disruption, for example at a temperature of between 4° C. and 37° C., preferably 15° C. and 30° C., more preferably between 18° C. to 27° C. A temperature of between 21° C. and 23° C. is particularly preferred.

Alternatively, the second disruption is carried out through proteolytic digestion, as is known in the art. This method is particularly useful if the disrupted/eluted polypeptides are to be assessed by MS.

Again, in these embodiments, a skilled person can readily determine an appropriate time period to use in order to disrupt the interaction of the binding agents with their bound polypeptides. By way of example, the second disruption may be carried out for 1 to 30 minutes, preferably 5 to 20 minutes, more preferably about 10 minutes.

In some embodiments of the invention, the second disruption conditions (or the more harsh or stringent conditions as described above) can be used alone in the disruption step (vii).

The downstream analyses discussed above can help to obtain further information in relation to the polypeptide of interest or in relation to binding agents that bind to (and in particular are specific for) the polypeptide of interest. As discussed above, the polypeptide of interest includes polypeptides that a person carrying out the method of the present invention wishes to find a specific binding agent for, and generally information regarding the polypeptide is known beforehand. However, there are also circumstances where the method of the present invention as described in steps (i) to (iv) identifies a polypeptide that a person then takes an interest in. For example, if a person generates results such as those shown in the graph of FIG. 1, in which the solid line peaks to the right of the specific peak of FIG. 1 are identified as cross-reactive or non-specific binding agents, then analysis of the nature of the cross-reactive polypeptides (the nature of which the person may well not be aware of) can be carried out.

Such analysis can be carried out in any appropriate way (including the use of appropriate methods of the invention to analyse the fraction or fractions containing the cross-reactive polypeptide). For example, such analysis can be carried out by analysing the chromatogram formed in the one or more fractions where the cross reactive peaks have formed and determining the polypeptides in those fractions that are present in a high abundance. Such analysis can alternatively be carried out by carrying out downstream steps (v) to (viii) discussed above but with respect to the newly identified polypeptide rather than the polypeptide of interest.

The only information available to the person analysing the cross-reactive nature of a binding agent may be that the newly identified polypeptide binds to the binding agent, in which case the contacting step (vi) could be carried out with that same binding agent. Once the newly identified polypeptide has been released after step (vii), analysis of this polypeptide can be carried out either by using MS, as discussed above, or through contacting the released polypeptide with a plurality of binding agents attached to one or more solid supports and detecting the binding of the polypeptide to the binding agents (binding agent array). The results from the previous analysis (carried out at step (iv)) can be compared against the newly generated data and by doing so, the skilled person would have a more precise understanding of the cross-reactive nature of the binding agent.

In some embodiments of the invention the separation step (i) can itself comprise multiple steps. Thus, in one embodiment the separation step (i) is comprised of the following steps:
(i.a) separation of polypeptides in the mixture into a plurality of fractions;
(i.b) contacting a first aliquot of two or more of the fractions with a plurality of different binding agents attached to one or more solid supports and detecting the binding of the polypeptides to the binding agents in each fraction;
(i.c) determining one or more fractions which are enriched for a particular polypeptide of interest;
(i.d) separating the enriched fractions into a plurality of fractions.

These steps (i.a) to (i.d) can be carried out as described elsewhere herein where the same or equivalent steps are used in other methods. Preferably antibody arrays are used as the binding agents attached to the solid supports in step (i.b).

Step (i.d), i.e. the step of separating the enriched fractions into a plurality of fractions, can be carried out by any appropriate technique. A preferred technique would be to use the step of contacting the one or more fractions which are enriched for a particular polypeptide of interest with a binding agent to said polypeptide of interest attached to one or more solid supports (such a step is also described herein as step (vi) in the various methods). More preferably this step would be carried out by immunoprecipitation (IP) using an antibody attached to a solid support (or solid phase) as described elsewhere herein. In such embodiments typically only a single type of binding agent/antibody is attached to the solid support.

In these embodiments, where a solid support is used for step (i.d), an additional step which can advantageously be used in some embodiments is a further separation into bound and unbound fractions. This can conveniently be done by removing the solid support to one fraction (the bound fraction) and then taking the supernatant into another fraction (the unbound fraction). The bound fraction will contain polypeptides which are bound to the binding agent of interest that is attached to the solid support, and the unbound fraction will contain the remaining polypeptides in the mixture, i.e. the polypeptides which are not bound to the binding agent of interest that is attached to the solid support.

In further embodiments the polypeptides in the bound and the unbound fractions can then be analysed. A preferred way of doing this would be to conduct parallel binding agent (e.g. antibody array) and MS analysis as described elsewhere herein (for example as described for steps (ii) and (iii) in the methods of the invention) on the bound fractions and/or the unbound fractions. The parallel binding results and the MS results can then optionally be correlated as described elsewhere herein (for example as described for step (iv) of the methods of the invention).

Such embodiments, where the separating/separation step (i) itself comprises multiple steps such as the steps (i.a) to (i.d) described above, are conveniently used when the starting number of fractions is high, e.g. 10 or more fractions (or higher numbers of fractions as described elsewhere herein). For example, it can be noted that the steps (i.a) to (i.d) do not require a parallel binding agent and MS analysis (only the use of a binding agent is specified) and such steps can conveniently be used to select lower numbers of fractions and/or to reduce the complexity of the fractions (e.g. in terms of polypeptide number and content), to be put through the parallel binding agent and MS analysis at steps (ii) and (iii) of the methods of the invention as described elsewhere herein.

In some embodiments the steps (i.a) to (i.d) are repeated one or more times, for example to allow further reduction in the number of fractions and/or the complexity of the fractions (e.g. in terms of polypeptide number and content) to put through the parallel binding agent and MS analysis. Such repeated steps are generally carried out in the same order as the earlier steps, i.e. (i.a), (i.b), (i.c) then (i.d).

Finally, the present invention provides a further method for analysing a mixture of polypeptides comprising the steps of:
(A) separating the polypeptides in the mixture into a plurality of fractions;
(B) contacting a first aliquot of two or more of the fractions with a plurality of different binding agents attached to one or more solid supports and detecting the binding of the polypeptides to the binding agents in each fraction;
(C) determining one or more fractions which are enriched for a particular polypeptide of interest;
(D) contacting an aliquot of one or more of the enriched fractions of step (C) with a binding agent to said polypeptide of interest attached to one or more solid supports;
(E) detecting the binding of polypeptides to the binding agent by mass spectrometry.

These steps (A) to (E) can be carried out as described elsewhere herein where the same or equivalent steps are used in other methods. For example, separation step (A) in the method above can correspond to the separation step (i) of other methods as described elsewhere herein, contacting step (B) in the method above can correspond to the contacting step (ii) of other methods as described elsewhere herein, determining step (C) in the method above can correspond to the determining step (v) of other methods as described elsewhere herein, the binding agent step (D) in the method above can correspond to the binding agent step (vi) of other methods as described elsewhere herein, detecting step (E) in the method above can be carried out by any MS detection method for example as described for the assessing step (iii) of other methods as described elsewhere herein. Again as described elsewhere herein the polypeptides need to be digested prior to MS analysis and this can conveniently be carried out by on-bead trypsin digestion or release of polypeptides followed by digestion as described elsewhere herein.

Step (D), i.e. the step of contacting one or more of the enriched fractions can be carried out by any appropriate technique using any appropriate binding agents. More preferably this step would be carried out by immunoprecipitation (IP) using an antibody attached to a solid support (or solid phase) as described elsewhere herein. In such embodiments typically only a single type of binding agent/antibody is attached to the solid support.

Thus, preferably step (D) is an IP step and step (E) is an MS step. Thus, steps (D) and (E) together describe a process of IP-MS. IP-MS techniques are known in the art and when carried out with a single antibody on a total native cell sample/lysate generally contain several hundred proteins, making analysis of which of these proteins binds directly to the antibody impossible. Such standard methods of IP-MS are therefore not useful to assess antibody specificity. Surprisingly the methods of the present invention in which IP-MS is carried out on an enriched fraction prepared using the fractionation and array analysis as described herein (i.e. steps (i) and (ii) and (v) of the methods as described herein, or steps (A), (B) and (C) of the method described in this embodiment) show extremely high purity. This is illustrated in FIGS. 11 and 12 where it can be seen that in contrast to prior art IP-MS methods (which contain several hundred proteins), the MS analysis step of the present methods (step (E) above or step (viii) in other MS involving embodiments) gives rise to the detection of only a few proteins.

This is particularly the case when stable isotope labelling with amino acids in culture (SILAC labelling) is used. Indeed SILAC labelling (or stable isotope labelling carried out on live cells as a form of metabolic labelling) can preferably be used with any of the methods of the invention described herein and it is particularly preferred when IP (or other binding agent)-MS techniques are used. Thus, in preferred embodiments of this aspect, SILAC labelling of cells is carried out prior to step (A) or step (i) of the methods described herein. Methods for conducting SILAC (or metabolic labelling with isotopes) are well known and described in the art, and an exemplary method is described in the Examples.

It is also clear from the results shown in FIGS. 11 and 12 that MS results/data from a parallel binding agent and MS analysis (e.g. parallel steps (ii) and (iii) as described herein) is not necessary for this aspect of the invention, although such a parallel MS step (e.g. step (iii) as described herein) can be carried out in the above methods as an additional step to steps (A) to (E) in parallel with step (B).

As can be seen from the Examples and FIGS. 11 and 12, the binding of polypeptides as detected in step (E) (or step (viii) in other MS involving embodiments) can be used to confirm specific binding of a binding agent (e.g. antibody) of interest to its target polypeptide.

In other embodiments of this aspect, the method further comprises the steps of:
(F) disrupting (or eluting) the binding agents of step (D) from the associated polypeptides;
and
(G) contacting the released polypeptides with a plurality of binding agents attached to one or more solid supports and detecting the binding of the polypeptides to the binding agents.
   In other embodiments of this aspect, the method further comprises the steps of:
(H) detecting unbound polypeptides in an aliquot from step (D) by mass spectrometry;
(I) detecting unbound polypeptides in a second aliquot from step (D) with a plurality of binding agents attached to one or more solid supports and detecting the binding of the polypeptides to the binding agents.
   In other embodiments of this aspect, the method further comprises the steps of:
(J) correlating results from step (B) with step (G) and/or step (I).

Such steps (H) to (J) can either be carried out in addition to steps (F) and (G), i.e. the method will involve all of steps (A) to (J), or steps (H) to (J) can be carried out after steps (A) to (D), i.e. steps (F) and (G) are not carried out. Alternatively, steps (A) to (D) can be followed by steps (F) and (G) and (J) and steps (H) to (I) are not carried out. In such methods the method steps can be carried out in any appropriate order. For example, in methods where both steps (H) to (J) and steps (F) and (G) are carried out then steps (H) to (J) can be carried out before, at the same time, or after steps (F) and (G), and vice versa.

Preferably antibody arrays are used as “the plurality of binding agents attached to one or more solid supports” in the above aspects.

Steps (A) to (E) of this method provide MS analysis and data on the polypeptides which are bound to a binding agent of interest (in step (D)) attached to a solid support (i.e. bound fraction MS analysis).

Steps (F) and (G) of this method provide binding agent array (e.g. antibody array) analysis and data on the polypeptides which are bound to a binding agent of interest (in step (D)) attached to a solid support (i.e. bound fraction array analysis or bound fraction antibody array analysis).

Step (H) of this method provides MS analysis and data on the polypeptides which are not bound to the binding agent of interest (in step (D)) attached to a solid support (i.e. unbound fraction MS analysis).

Step (I) of this method provide binding agent array (e.g. antibody array) analysis and data on the polypeptides which are not bound to the binding agent of interest (in step (D)) attached to a solid support (i.e. unbound fraction array analysis or unbound fraction antibody array analysis).

Thus, in these embodiments, a solid support is used for step (D), and an additional step which can advantageously be used in some embodiments is a further separation into bound and unbound fractions. This can conveniently be done by removing the solid support to one fraction (the bound fraction, enriched fraction) and then taking the supernatant into another fraction (the unbound fraction, depleted fraction). The bound fraction will contain polypeptides which are bound to the binding agent of interest that is attached to the solid support, and the unbound fraction will contain the remaining polypeptides in the mixture, i.e. the polypeptides which are not bound to the binding agent of interest that is attached to the solid support.

In further embodiments the polypeptides in the bound and the unbound fractions can then be analysed. A preferred way of doing this would be to conduct parallel binding agent (e.g. antibody array) and MS analysis as described elsewhere herein (for example as described for steps (ii) and (iii) in the methods of the invention) on the bound fractions and/or the unbound fractions. The binding results and the MS results can then optionally be correlated as described elsewhere herein (for example as described for step (iv) of the methods of the invention). Alternatively, the binding results from the bound and the unbound fraction can be compared/correlated and/or the MS results from the bound and the unbound fraction can be compared/correlated. For example, MS analysis of the enriched fraction provides the sequence for the protein(s) bound by the antibody target. MS analysis of the depleted fraction provides information about the proteins that were not bound. By correlating the results, one can quantify the enrichment obtained with the antibody used in step (D). By analyzing both fractions with an antibody array, one can detect reduction in the signal of other antibodies in the array that recognize the same target as the binder used in step (D).

For correlation step (J) one would generally correlate/compare the results obtained after the first array analysis of the fractions (B) and those obtained after array analysis of the enriched fraction (G) and (I) which is array detection of the depleted fraction. To put another way, the first array analysis will provide information about the content of a given antibody target in the fraction before you do the IP step (D). You next analyze the enriched fraction (G) and finally the depleted fraction (I). The array contains the antibody used for IP, and the results from step (B) serve as reference. If the signal in the depleted fraction is 30% of that measured in step (B), the depletion was 70%. If the array contains other antibodies to that protein, a similar drop is expected. In the enriched fraction, it is expected to see that beads with antibodies to the same protein have signal, and no signal on beads that have antibodies to other proteins.

Thus, the methods of this embodiment can be used to determine enrichment and depletion of the bound polypeptides and in turn provide an assessment of antibody specificity. For example, antibody array analysis may identify five antibodies that bind to a particular target. Rather than carrying out MS on all of these to assess specificity (which is an expensive option), specificity can be assessed using the methods of this aspect. In this regard, one of the five antibodies can be used for IP (step D), after which the sample can be separated into the bound (enriched) and unbound (depleted) fractions as described elsewhere herein. The bound fraction will be enriched for the target protein and the unbound fraction will be depleted for the target of interest. A further binding agent array (antibody array) step can then be carried out on both the enriched and depleted fractions using all five of the antibodies, e.g. in separate reactions. If the same (or equivalent) loss of signal is observed with one of the four antibodies as was lost with the initial antibody used for IP then this shows that that antibody is also specific for the target protein of interest. If a different loss of signal is observed then this shows that the antibody binds to something other than the target protein.

Thus, study of the enriched fraction can show that the antibodies being tested can bind to a protein of interest (protein X). However, study of the depleted fraction provides addition important information as to whether the antibody can only bind to protein X or whether it binds to something else. If an antibody being tested binds to something in the depleted fraction then this shows that it is binding something other than protein X, i.e. that the antibody is not specific. The comparison of the data from the depleted and undepleted fraction can thus provide an assessment of specificity.

As described above, in preferred embodiments of the above aspect, SILAC labelling of cells is carried out prior to step (A) of the methods described herein.

Other features and preferred embodiments of these methods are as described elsewhere herein for the other methods of the invention. In particular, chemical labelling of polypeptides, e.g. with biotin, prior to the separation step (A) is preferred. More preferred is a combination of SILAC and chemical labelling prior to the separation step (A). In other preferred embodiments, the sample (s) is subjected to harsh treatment, e.g. denaturation, e.g. SDS-heat denaturation, to disrupt protein complexes prior to the separation step (A). In other preferred embodiments, separation is by gel electrophoresis as described elsewhere herein.

As this method of the invention involves IP-MS, a further preferred embodiment is one wherein the MS analysis is multiplexed using addressable bar codes (i.e. barcodes that are traceable to a single capture reaction, e.g. identifying a single binding agent or antibody). Any addressable bar code can be used, examples of which would be well known to a person skilled in the art. Preferably the addressable bar code is a stable isotope (e.g. the use of different SILAC labels or other isotope labels). Alternatively, the addressable bar code can be a physical parameter (for example protein size) specific for proteins in a certain fraction. In this embodiment for example if fraction 1 contains proteins smaller than 20 kDa and fraction 2 contains proteins larger than 40 kDa, then it is clear that any protein smaller than 20 kDa came from fraction 1 while those that are larger than 40 kDa came from fraction 2.

It is well known that many antibodies cross-react. Indeed, examples in the attached Figures show that antibody reactivity peaks are often detected that do not correlate with MS data for the intended target (see for example in FIG. 2C, FIG. 3A and FIG. 3C). Such reactivity peaks can represent cross-reactive antibodies, i.e. antibodies which bind to one or more additional protein targets to the intended target or perhaps antibodies that do not bind to the intended target protein but bind to a different target protein. The present invention also provides methods to identify the cross-reactive proteins, i.e. the other proteins that such antibodies are interacting with, by analysing the one or more fractions which correspond to this other reactivity peak using the methods of the invention. Thus, although the detection of antibody reactivity peaks that do not fit with the MS data may suggest that the antibody recognizes the “wrong” protein, the antibody may still be useful if that protein is identified and this can be done using the methods of the invention.

Shotgun MS (e.g. as used in step (iii) of the methods) is not as sensitive as binding agent (e.g. antibody) array analysis. Thus, a negative MS signal in the parallel binding and MS analysis steps (ii) and (iii) is not definitive evidence that the polypeptide of interest is not present in the fraction/sample, it may just be present at low abundance. Thus, analysis of such fractions using the methods of the invention can still be useful, for example providing a means to validate antibodies to low abundance proteins that are not detected by MS, e.g. shotgun MS.

Thus, we conclude that paired analysis of fractionated proteins with antibody arrays and MS using the methods of the invention as described herein is helpful to select antibodies that are likely to be specific and therefore worth the investment of more expensive and definitive downstream analysis by IP-MS. It is also clear that these methods will be useful to identify the targets of antibodies that cross-react. In paired array and MS analysis of fractions, one would identify an antibody reactivity peak that does not overlap with the MS signal. The antibody can then be used to immunoprecipitate the target from the enriched fraction for identification by IP-MS. Finally, some antibodies may show a reactivity peak when shotgun MS does not show a signal for the intended target. A negative MS signal is not definitive evidence for lack of protein expression. IP-MS is more sensitive than shotgun MS. Using the methods of the invention one can therefore identify targets of antibodies to low abundance proteins that are not detected by MS, e.g. shotgun MS.

### Combinations of Features

The above description describes numerous features of the present invention and in most cases preferred embodiments of each feature are described. It will be appreciated that each preferred embodiment of a given feature may provide a method of the invention which is preferred, both when combined with the other features of the invention in their most general form and when combined with preferred embodiments of other features. The effect of selecting multiple preferred embodiments may be additive or synergistic. Thus all such combinations are contemplated unless the technical context obviously makes them mutually exclusive or contradictory. In general each feature and preferred embodiments of it are independent of the other features and hence combinations of preferred embodiments may be presented to describe sub-sets of the most general definitions without providing the skilled reader with any new concepts or information as such.

Lists “consisting of” various components and features as discussed herein can also refer to lists “comprising” the various components and features.

Methods comprising certain steps also include, where appropriate, methods consisting of these steps.

All documents, papers and published materials referenced herein, including journal articles and published patent applications, are expressly incorporated herein by reference in their entireties.

The invention will now be further described in the following Examples and with reference to the figures in which:

FIG. 1 A schematic of a preferred method of the present invention. Cells from eight different cell types (represented by the petri dishes A to H) are lysed, and soluble proteins in cell lysates are labelled with amine- or thiol-reactive derivatives of a hapten such as biotin. Unreacted biotin is removed through the use of centrifugation filter units. The proteins are then denatured and separated by gel electrophoresis. During a typical separation, twelve fractions from the eight different samples labelled A to H (in this case cell types) are harvested, and transferred to a 96 well microplate. A liquid handling robot is used for precise transfer of liquid fraction aliquots from the master plate to two replicate plates. One of these two is supplemented with bead-based antibody arrays (marked “WMAP”, which stands for “Western Microsphere Affinity Proteomics”, in the figure). The plate is kept at 4 to 8° C. at constant agitation overnight in order to allow binding of the antibodies to the proteins. The plate is next subjected to centrifugation to pellet the beads in order to remove unbound protein and resuspended in washing buffer. After two washes, fluorescent streptavidin is added so that the biotin label on the captured proteins can be detected and so that the beads with captured protein can be separated from beads without captured protein. Finally, the beads are analysed using a flow cytometer.

The second plate is processed for analysis of peptides by mass spectrometry (marked “MS” in the figure). The sample processing used here involves the addition of beads with immobilised streptavidin to all liquid fractions. Biotinylated proteins bind indiscriminately to the beads. The beads are washed in order to remove unbound proteins and treated with trypsin in order to obtain peptides useful for mass spectrometry and analysed by liquid chromatography mass spectrometry.

The approach described above yields two sets of numerical data. The MS data (dashed line) represent the reference for validation of antibody specificity with respect to one protein specifically. Multiple dashed lines may be formed with respect to the same protein in each different cell type (i.e. each mixture of polypeptides), see for example FIG. 2. The WMAP data is presented as a solid line. The graph presenting both the WMAP data and the MS data would be produced for each antibody in the antibody array (WMAP data line) and the corresponding protein of interest (MS data line for the target protein which should be bound by the antibody). The proportion of overlap (correlation) of the signal curves from the WMAP data with that of the MS data provides a measure of the specificity of the antibody for the protein of interest (the specificity index).

FIG. 2 Algorithm used to assess sensitivity and specificity of antibodies. Plots of binding signal (fluorescence) intensity (antibody array signal) derived from WMAP (solid lines) analysis of protein captured by an antibody to the protein Akt1, and of relative abundance of Akt1 derived from MS (dashed lines) (y axis) for each of twelve size fractions (x axis), obtained using the method as described in FIG. 1. The polypeptide of interest was Akt 1. Cell lysates obtained from three different cell types were analysed, RT4 cells (squares), U2OS cells (circles) and HeLa cells (triangles).

A computer algorithm was used to identify the fraction with the highest signal intensity measured by MS (in this case fraction 10, hereafter referred to as the MS centre). The algorithm next calculates several indexes based on the antibody signal. The core index is the sum of the binding signal intensity from the antibody array analysis (antibody or binding agent array signal) measured in the fraction corresponding to the MS centre and the two immediate neighbouring fractions, i.e. the fraction each side of the MS centre (in this case fractions 9 to 11) divided by the sum of signal measured in all twelve fractions (total signal). The wide index (width index) is the sum of the binding signal intensity (antibody array signal) measured in the two immediate neighbouring fractions on each side of the MS centre (in this case fractions 8 to 12) divided by the total signal. The fractions that form the core and wide areas are shown in FIG. 2A. The signal index is the maximal signal intensity (from antibody or binding array analysis) with respect to all fractions analysed (for this case for all cell-types analysed) divided by the median signal. Maximal and median signal intensities are shown in FIG. 2A. The absolute signal intensity is the value for the maximal signal intensity measured by binding agent (antibody) array analysis. Finally, the algorithm determines the overall correlation between the signal values obtained with antibody array analysis (antibody array signal) and MS (this overall correlation can be referred to as the specificity index).

The antibody analysed in FIG. 2A has a core index of 0.86, a width index of 0.89 and a specificity index (correlation) of 0.98. FIGS. 2B to 2D show results obtained with three different antibodies to Akt1 in the same antibody array. FIG. 2B shows an antibody with a strong signal (maximum or absolute median fluorescence intensity (MFI) of greater than 20,000), but low core and wide indexes (i.e. a broad peak). This indicates lower specificity (lower specificity index) than the antibody shown in FIG. 2A. FIGS. 2C and 2D show antibodies that have an absolute (or maximum) MFI of below 3000, and in addition FIG. 2C has an extra peak in fraction 6. The antibodies of FIGS. 2C and 2D therefore have a low core, wide and signal indexes.

FIG. 3 Examples of antibodies identified as specific or cross-reactive. Plots of binding signal intensity (antibody or binding agent array signal) derived from WMAP (solid lines) and of relative abundance derived from MS (dashed lines) (y axis) for each of twelve size fractions (x axis), obtained using the method as described in FIG. 1. The polypeptides of interest were RBL2 (FIGS. 3A and 3B, a 128 kDa polypeptide) and beta-actin (ACTB) (FIGS. 3C to 3E, a 41 kDa polypeptide). Each plot represents a different antibody to the appropriate polypeptide of interest. Cell lysates obtained from three different cell types were analysed, RT4 cells (squares), U2OS cells (circles) and HeLa cells (triangles). FIGS. 3A and 3C show cross-reactive antibodies, as little overlap is seen between the antibody reactivity profile (solid lines) and the MS profile (dashed lines). FIGS. 3B, 3D and 3E show specific antibodies with a high level of overlap in antibody reactivity profile (solid lines) and the MS profile (dashed lines), indicating antibodies that are specific for RBL2 or ACTB. However, the antibody of FIG. 3E has a low absolute signal intensity (MFI of less than 1500) and so it can be concluded that the antibody in FIG. 3D is more sensitive than the antibody shown in FIG. 3E.

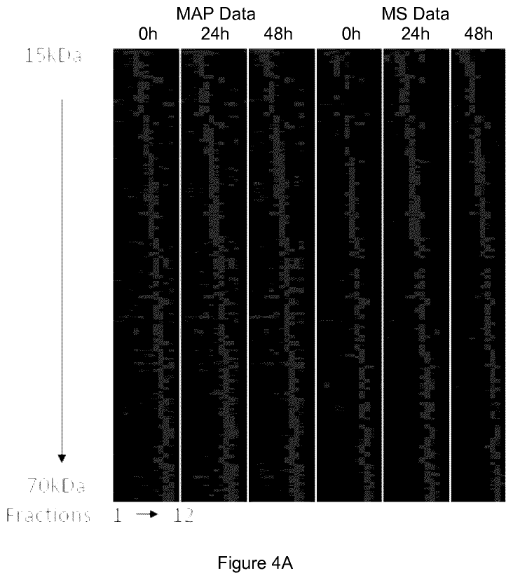

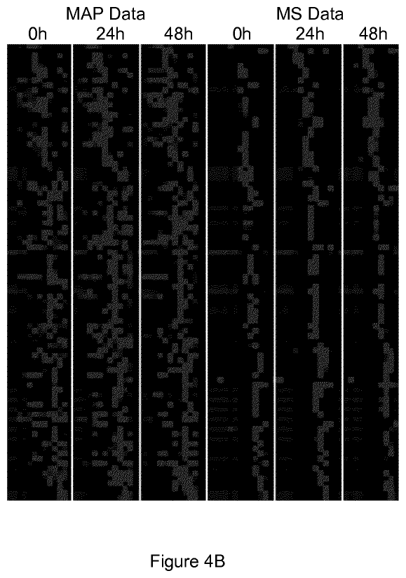

FIG. 4 Massive parallel assessment of antibody performance. Heatmaps showing reactivity profiles of hundreds of antibodies across fractions obtained from primary T cells immediately after isolation from blood or after 24 or 48 hours of in vitro activation with the mitogen Concanavalin A. 272 antibodies were analysed in FIG. 4A and 93 antibodies were analysed in FIG. 4B. The proteins (y-axis) were sorted in ascending order (top-down) according to predicted mass. With this formatting the distribution pattern of the proteins in the map is predictable. Thus, the signal maximum (grey pixels) for the smallest proteins is expected to appear in the top left corner and the signal is expected to distribute along the diagonal to the bottom right with increasing protein mass. Corresponding heatmaps for results obtained for the antibody targets by MS are shown in the right half of each map. The map in FIG. 4A shows results obtained with strict criteria (threshold) for antibody validation, specifically a specificity index of greater than 80%, a signal index of greater than 4, an absolute signal intensity of greater than 5000 and a core index of greater than 0.7. The strict criteria in FIG. 4A is evident from the similarity between the antibody reactivity profiles and the target distribution profiles as measured by MS. The antibodies shown in FIG. 4B did not satisfy the criteria (threshold) used in A, but satisfied less strict criteria, specifically a specificity index of between 70 and 80%, a signal index of between 3 and 4, an absolute signal intensity of greater than 2000 and a core index of less than 0.7. The pattern of signal distribution is more complex than in FIG. 4A and less similar to the MS profiles.

FIG. 5 Targeted immunoprecipitation followed by antibody array analysis. Proteins from different subcellular compartments in CD4+ T cells were separated and analysed with antibody arrays and flow cytometry. The line charts (FIG. 5A) show signal from biotinylated protein captured by indicated specificity, i.e. anti-CD3e or anti-CD247 (y-axis, log scale) plotted against SEC fraction number (1 to 24). The sub-cellular locations analysed were (1) cytosol, (2) organelles, (3) nucleus and cytoskeleton and (4) membrane. Fractions containing high levels of membrane-associated targets for anti-CD3e and the associated protein CD247 (CD3zeta), were identified (longer arrows). Antibodies were then used to immunoprecipitate their respective targets from a separate aliquot of the fraction. After overnight incubation, the beads were first subjected to very mild elution conditions (1% Tween in phosphate buffered saline at 22° C., shaking for 30 minutes) and then to harsh elution (0.1% sodium dodcecyl sulfate solution at 95° C.). Eluted proteins were next analysed with bead-based antibody arrays (FIG. 5A, bottom right panel). The bar plots show signal intensity for the ten microsphere subsets with the highest signal with respect to CD247 capture (FIG. 5B) and CD3e capture (FIG. 5C) (log scale).

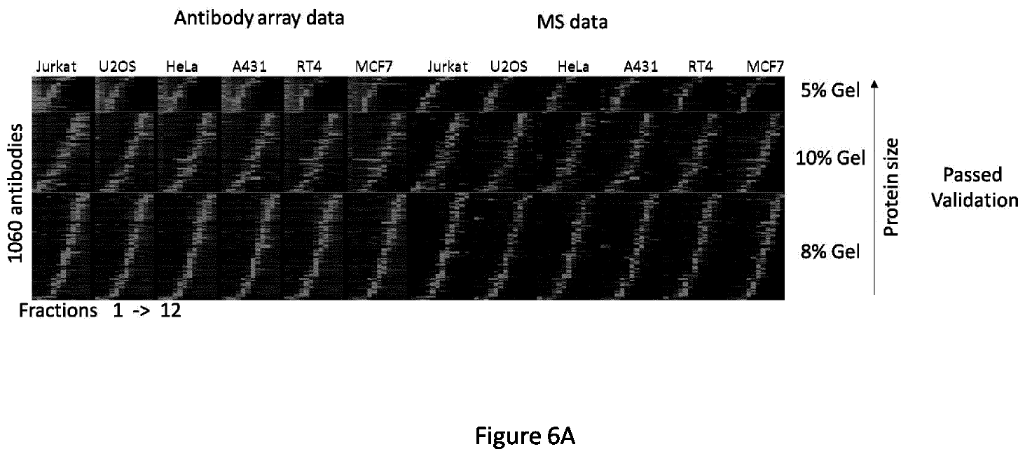

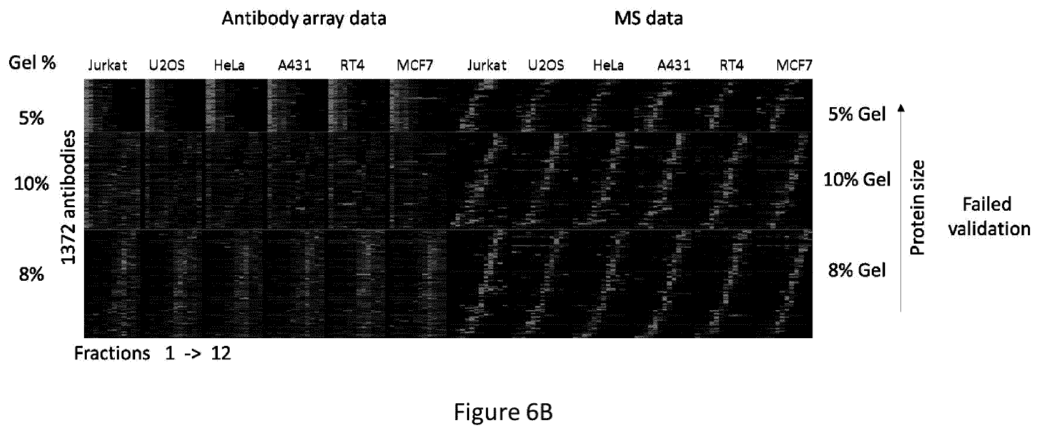

FIG. 6 Reactivity patterns of antibodies that passed or failed validation on basis of overlap in chromatograms. The heatmaps show binder chromatograms for antibodies (left half) alongside MS chromatograms for the intended antibody targets. Proteins from six cell lines (Jurkat, U2O5, HeLa, A431, RT4, MCF7) were labelled with biotin and separated by preparative gel electrophoresis (Gelfree-8100). Three gels with different separation ranges were used (5%, 8% or 10% acrylamide). The proteins were next analyzed as outlined in FIG. 1. The x- and y-axis in each map corresponds to Gelfree fraction number, and antibodies/proteins, respectively. The largest and smallest proteins appear at the top and bottom in each map, respectively. Since protein mass increases along the y-axis as well as with fraction number (x-axis), the expected pattern is a continuum of “bands” from the lower left to the upper right in each map. In FIG. 6A the map shows reactivity patterns of 1060 antibodies that passed criteria for signal to noise (signal index) and peak position (core index) set by a computer algorithm. The similarity between data obtained with antibodies and MS, respectively, can be noted. In contrast, FIG. 6B shows results obtained with antibodies that failed to meet the same criteria. The results obtained with these antibodies do not recapitulate the MS data.

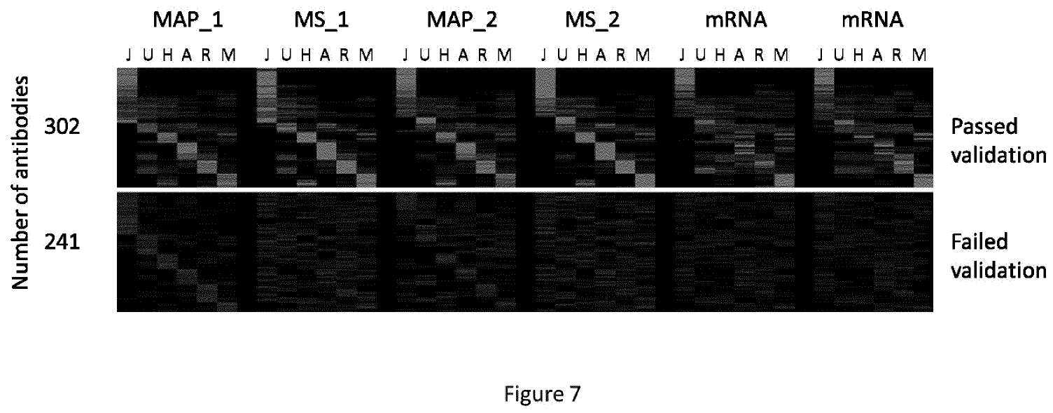

FIG. 7 Reactivity patterns of antibodies that passed or failed validation on basis of overlap in chromatograms and correlation. The heatmaps show relative protein levels measured in six cell lines (same sequence as in FIG. 6) by antibody array analysis and MS, alongside transcriptomics data (mRNA) retrieved from two published datasets. The original data set contained 12 data points (from 12 fractions) per antibody (antibody data) and antibody target (MS data). Here, the sum of five data points centered around the maximum value were used to calculate a single value for protein abundance (a wide index). All antibodies shown in the figure passed criteria for signal to noise (signal index) ratio of 4 or more. The 302 antibodies in the top map also passed criteria for overlap with the MS chromatogram (4× median) as well as criteria for correlation between antibody and MS data (correlation of 0.7). The similarity in patterns observed for antibody array data (MAP) and MS data can be noted. It can also be noted that a similar pattern is observed for the mRNA data. The mRNA data represent an independent control since they were retrieved from an article published by a different laboratory. The lower heatmap was organized according to the relative abundance measured with antibodies in experiment 1. Part of the pattern was reproduced in experiment 2 (a replicate experiment). However, there is no corresponding pattern in the MS or mRNA data. The antibodies therefore failed validation.

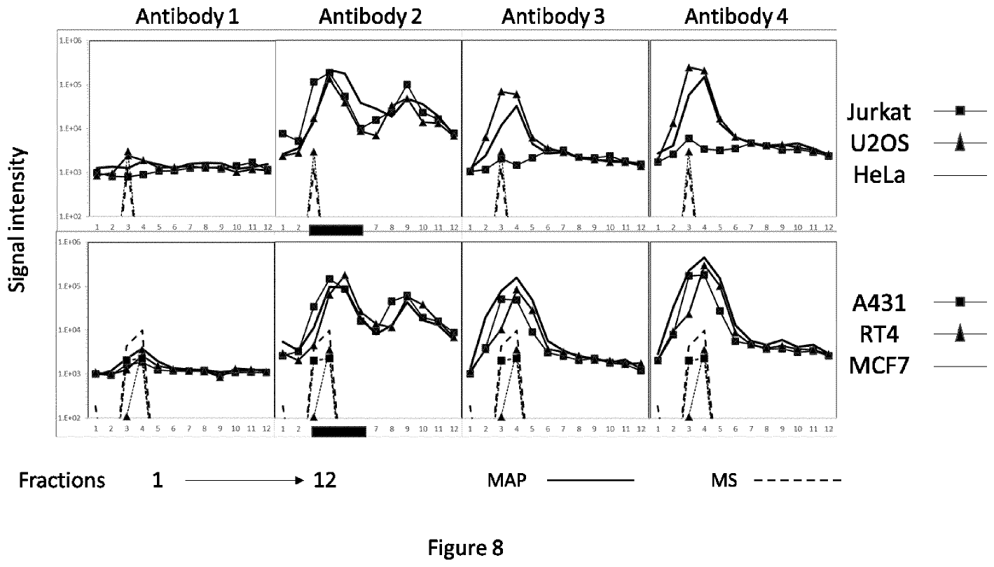

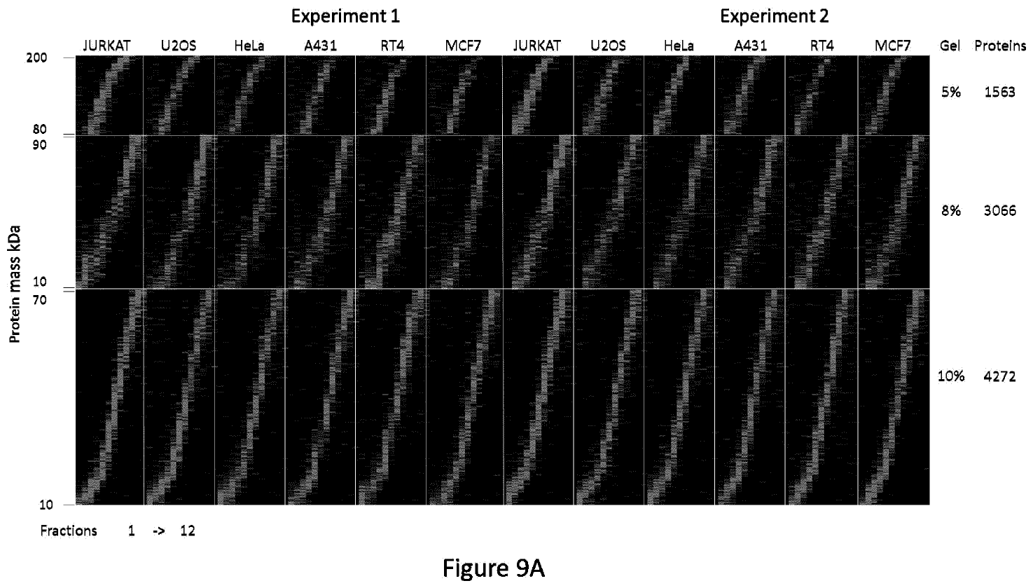

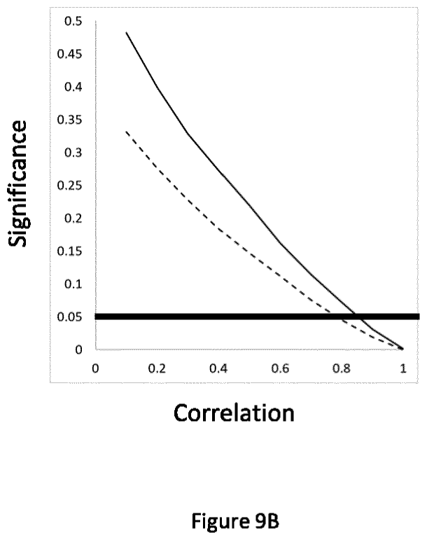

FIG. 8. Correlation of results obtained with antibody array analysis and MS. The charts show signal intensity (y-axis, log scale) obtained with four different antibodies to CDKN1A (solid lines) plotted against fraction number (Gelfree preparative gel electrophoresis, 10% gel). The dashed lines show MS signal intensity for CDKN1A in the same fractions. Antibody 1 failed the criterion for sensitivity (signal index) since the strongest signal was less than four-fold higher than the median. Antibody 2 bound two targets, but passed the criterion for chromatogram overlap (peak position, core index) since the tallest antibody reactivity peak did not deviate by more than one fraction from the signal maximum of CDKN1A as determined by MS (dashed lines). However, antibody 2 failed to meet the specificity criteria since the correlation with MS data was lower than the threshold of 0.7 for the reactivity profile (all data points) and relative protein abundance (sum of datapoints in the wide index, corresponding to five datapoints centered around the maximum signal). Antibodies 3 and 4 passed all criteria. The correlation was higher than 0.9 which yields a statistical significance better than p=0.05 (see legend to FIG. 9).

FIG. 9 Assessment of significance of correlations. The heatmap in FIG. 9A shows 8901 MS chromatograms from two experiments. Six human cell lines were cultured in the presence of amino acids with stable isotopes. The cells were lysed, and proteins were labelled with biotin and separated by preparative gel electrophoresis. Labelled proteins in each lysate were separated using three gels with different separation range (5%, 8% or 10% acrylamide). The proteins were processed and analyzed by shotgun MS analysis as described in FIG. 1. The proteins in the dataset were sorted according to the type of gel used for separation and then in descending order according to predicted mass. To assess random correlation, the values in each row of data from each experiment were correlated to those in the row below. The line chart (FIG. 9B) shows frequency (y axis) of random correlations in datasets obtained by analyzing fractions obtained by gel electrophoresis by MS. Spreadsheet functions were used to determine the frequency of data series with indicated correlations in the datasets shown in FIG. 9A. The horizontal line indicates a significance of 0.05. Random correlations were determined by correlation of data in neighboring rows.

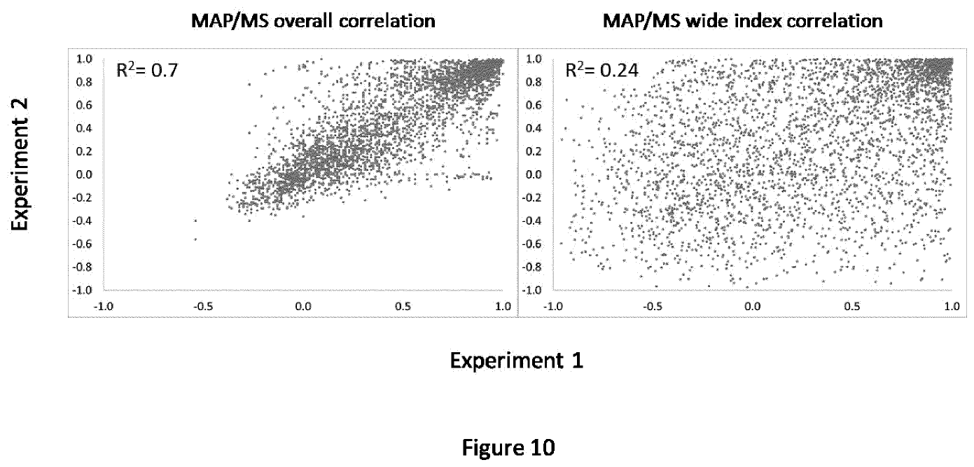

FIG. 10. Correlations for all data points measured across a series of fractions are more reproducible than correlations for relative protein abundance.

The dot plots show distribution of correlations between results obtained by antibody array analysis and MS in two experiments. Arrays with content of 2406 antibodies were used to analyze 12 fractions of cellular proteins obtained by gel electrophoresis. An aliquot of the same fractions were analyzed by shotgun MS. Two types of correlations were performed in each experiment: The MAP/MS profile correlation is the correlation of all signal values obtained with MAP and MS, respectively in fractions 1-12 (overall correlation). Relative protein abundance was measured as the sum of signal values in five fractions centered around the fraction with the maximal signal in fractions from each cell type (wide index). The R² values represent squared Pearson correlations.

FIG. 11 Downstream analysis

The line plot shows signal intensity (y-axis, log scale) for beta-actin plotted against fraction number. Solid lines indicate streptavidin fluorescence intensity measured by antibody array analysis. Dashed lines show MS signal intensity measured for actin-beta in the same fractions. Jurkat cells were cultured in media containing isotope-labelled amino acids. The cells were lysed, and the proteins were labelled with biotin, denatured and separated according to size using a Gelfree 8100 instrument for preparative gel electrophoresis. Twelve fractions were incubated with a bead-based antibody array. The arrays were washed, labelled with fluorescent streptavidin and analyzed by flow cytometry. The plot shows signal intensity measured for a subset of beads coupled with anti-beta actin (ACTB). The strongest signal was observed in fraction 8 MS data confirmed that this was the fraction most highly enriched for beta-actin. Beads with anti-beta-actin were used to capture the antibody target from 0.1 ug of protein from fraction 8. The beads were subjected to on-bead trypsin digestion and the peptides were sequenced by MS. The bar graph in the lower left hand panel shows MS signal intensity for indicated proteins that contained isotope-labelled amino acids. The signal for beta-actin was almost hundred times higher than those measured for any other sample-derived protein. The bar graph in the lower right panel shows MS signal intensity for proteins that did not contain SILAC label. These proteins therefore represent contamination. Note that gamma actin (ACTG1) is on the list of contaminants. This protein is highly homologous to beta-actin, and if this protein was not identified as contamination, one would have falsely assumed that the anti-beta-actin antibody cross-reacted with gamma-actin.

FIG. 12 Downstream analysis

The line plot shows streptavidin fluorescence intensity (y-axis, log scale) plotted against fraction number. Jurkat cells were cultured in media containing isotope-labelled amino acids. The cells were lysed, and the proteins were labelled with biotin, denatured and separated according to size using a Gelfree 8100 instrument for preparative gel electrophoresis. Twelve fractions were incubated with a bead-based antibody array. The arrays were washed, labelled with fluorescent streptavidin and analyzed by flow cytometry. The plot shows signal intensity measured for a subset of beads coupled with anti-Rel A (RELA). The strongest signal was observed in fraction 8. Beads with anti-Rel A were used to capture the antibody target from 10 ug or 1 ug of protein from fraction 8. The beads were subjected to on-bead trypsin digestion, and the peptides were sequenced by MS. The bar graph in the lower left hand panel shows MS signal intensity for indicated proteins that contained isotope-labelled amino acids. When 1 ug of protein was used as source, RELA was the only protein detected. When 10 ug was used, there was also a signal from HSPA2, but the signal from RELA was more than 10 times stronger. The bar graph in the lower right hand panel shows MS signal for proteins without stable isotopes. These represent contamination. Many of these have far higher signal intensity than RelA, and several are proteins that are found in Jurkat cells. Without SILAC labeling it would therefore be difficult to exclude that they represent cross-reactivity of the RELA antibody.

### EXAMPLES

### General Materials and Methods

### Covalent Coupling of Protein G and Fluorescent Dyes to Particles to Form Colour-Coded Particles

Polymer particles (6 or 8 μm, PMMA, amine-functionalised, www.Bangslabs.com) were reacted with sulfo-SPDP (Sigma) (3 mg per gram of particles) at 10% solids in PBS 1 mM EDTA 1% Tween 20 (PBT) for 30 minutes at 22° C. under constant rotation. The particles were pelleted by centrifugation at 500 g for 5 minutes, washed once in PBT, and reduced with 5 mM TCEP (Sigma) for 20 minutes at 37° C. Particles were pelleted, washed once in 100 mM MES pH 5 (MES-5) and resuspended at 10% solids in MES-5. Protein G (Fitzgerald Industries) was dissolved at 5 mg/ml in PBS, reacted with 100 ug/ml Sulfo-SMCC (30 minutes, 22° C.) and transferred to MES-5 using G-50 spin columns. Two milligrams of protein G-SMCC was added per gram of particles under constant vortexing. After 30 minutes of rotation at 22° C., particles were resuspended in 100 mM MES pH 6 containing 1 mM EDTA 1% Tween 20 with 1 mM TCEP (MES-6-TCEP) and stored at 4° C. until labeling with fluorescent dyes. Particles were stable for several weeks in this buffer. Fluorescent labeling was performed by incubating equal aliquots of particles at 1% solids with a serially diluted fluorescent maleimide for 30 minutes at 22° C. Differently labeled aliquots were washed twice in MES-6-TCEP and split in new aliquots, each of which were reacted with different concentrations of the next dye. The sequence used here was Alexa 488, Alexa 647, Pacific blue (all in MES-6) and Pacific Orange (PBT). The starting concentrations were 50 ng/ml for Alexa 488 and Alexa 647, 25 ng/ml for Pacific Blue, and 500 ng/ml for Pacific Orange. The dilutions were between two and three-fold. This method enables populations of particles to be prepared, each with a different colour code that can be distinguished from each other for example by an appropriate flow cytometer.

### Binding of Antibodies to Color-Coded Particles

Before coupling of antibodies, particles were suspended in PBS casein block buffer (www.piercenet.com) for 24 hours at 4° C. Polyclonal antibodies (2 μg for 10 μl of 10% bead suspension) were added to particles suspended in casein-PBS block buffer. The particles were rotated for 30 minutes at 22° C. Polyclonals from rabbit and goat can be coupled directly to particles with protein G. For binding of mouse monoclonal antibodies, particles were first reacted with subclass-specific goat-anti-mouse IgG Fc (Jackson Immunoresearch), then with the mAbs. After three washes in PBT, a small aliquot of all particles was added to a single vial and labeled with phycoerythrin (PE) conjugated anti-mouse, anti-rabbit and anti-goat IgG to assess antibody binding. The particles were resuspended in PBT with 50% trehalose and 40 μg/ml non-immune gamma globulins from goat and mouse to prevent crossover of specific antibodies between particles. Particles with different antibodies were mixed and stored frozen in aliquots at −70° C. Control experiments showed that freezing did not affect performance of the arrays (not shown). Approximately 5% of the particle populations were coupled to polyclonal non-immune immunoglobulins mouse and goat IgG and used as reference for background.

### Cells

Human leukocytes were obtained from buffy coats from healthy blood donors. CD4 T cells were isolated using a RosetteSep kit (STEMCELL technologies Inc.). The U2OS and RT4 cell lines were obtained from ATCC. The cell lines HeLa (ovarian carcinoma) U2OS and RT4 were cultured in RPMI with 20 mM HEPES and 5% fetal bovine serum.

### Cell Lysis and Labeling of Proteins

For separation by gel electrophoresis, cells may be lysed in a solution containing 140 mM NaCl, 30 mM HEPES pH 7.4, 0.3% Sodium Dodecyl Sulphate (SDS) and 1 Mm TCEP. Lysed cells were immediately heated to 90° C. for 10 min. Total cell lysates prepared for separation of proteins under native conditions, are typically prepared by lysing of cells in a solution containing 140 mM NaCl, 30 Mm HEPES pH 7.4, 1% dodecyl maltoside, and commercially available cocktails of inhibitors for proteases and phosphatases. Subcellular fractions may be prepared using commercially available kits from e.g. Thermo Scientific. For covalent labeling of proteins, cell lysates are supplemented with amine-reactive biotin (e.g 500 μg/ml biotin-PEO-4-NHS) or thiol-reactive biotin (e.g. biotin-PEG2, maleimide) and the samples are incubated for 20 minutes at 22° C. Free label was removed through the use of centrifugation filter units.

### Gel Electrophoresis

Biotinylated cellular proteins were supplemented with Sodium Dodecyl Sulfate (SDS) and heated. The denatured proteins were next subjected to gel electrophoresis using a GELFREE® 8100 instrument (Expedeon Ltd, UK) to separate the proteins into liquid fractions according to size using conditions recommended by the manufacturer. During a typical separation, twelve fractions from up to eight samples were harvested, and transferred to a 96 well microplate. A liquid handling robot (CyBio® SELMA) was used for precise transfer of liquid fraction aliquots from the master plate to two replicate plates.

The difference between a Western Blot and carrying out electrophoresis using the commercially available instrument Gelfree® 8100 is that this instrument yields liquid fractions with size separated proteins. The instrument is used with gel cassettes, and running buffers according to the manufacturer's instructions. Proteins are loaded into cassettes useful for parallel separation of proteins from up to eight samples. During electrophoretic separation, proteins migrate through a gel, and liquid fractions containing proteins with a narrow size range are collected at different time points in separate sample collection chambers. Small proteins migrate fast and are collected first. The manufacturer recommends the use of 10% Tris-Acetate gels for separation of proteins with a mass of 15-100 kDa, 8% gels for resolution between 35-150 kDa and 5% gels for resolution between 75-500 kDa.

Incubation of Labeled Proteins with Antibody Arrays

Mixtures of colour-coded particles with antibodies bound thereto were thawed, pelleted and resuspended in PBS casein block buffer (Pierce®) with 40 μg/ml of mouse and goat gammaglobulins. Ten microliters of the suspension was added to each well of one of the replicate plates (polypropylene 96 well PCR plates, from Axygen® Inc). Biotinylated proteins (25 μl) were added by a liquid handling robot as described above, the wells capped and plates constantly agitated overnight at between 4 and 8° C. Particles were then pelleted by centrifugation washed at least two times in PBT and labeled with 10 μl streptavidin-phycoerythrin (PE) (2 μg/ml in PBS with 2% fetal bovine serum, streptavidin-PE was obtained from Jackson Immunoresearch (www.JiREurope.com)). Labeled particles were washed twice in PBT, resuspended in 200 μl PBT and analysed using a flow cytometer.

### Flow Cytometry

An LSRII flow cytometer was used to collect data. The flow cytometer is used to read the microsphere fluorescent colour-codes and to measure fluorescence from the streptavidin reporter molecule. Pacific Blue and Pacific Orange were excited by a 405 laser using 450 and 530 band pass filters, respectively. Alexa 488 and Phycoerythrin (PE) were excited by a 488 nm laser and light collected through 530BP and 585BP filters, respectively. Alexa 647 was excited by a 633 nm laser and light collected through a 655BP filter.

### Mass Spectrometry

Biotinylated proteins in the second replicate plate were captured onto agarose beads covalently coupled with streptavidin. Following repeated washing steps in salt- and detergent-free media, the particles were suspended in a solution containing the proteolytic trypsin to facilitate digestion of the captured proteins. Peptides were solubilized in 0.1% formic acid and loaded onto a nano-liquid chromatography column interfaced directly into a mass spectrometer (liquid chromatography mass spectrometry).

### Data Analysis

Flow cytometry data were processed through R script analysis (Stuchly et al., 2012, Cytometry Part A 81 (2), 120-129). Raw mass spectrometry data files were processed with MaxQuant in order to identify proteins. These yield two sets of numerical data which can be correlated, where the MS data represents the reference for assessment of antibody specificity. An example of the type of data obtained is shown in FIG. 1, where the dotted lines represent the MS data and the solid lines represent the flow cytometry (antibody binding) data. The proportion of the signal that overlaps with that measured by MS is considered as the specificity index. Determination of specificity index, core index, wide index, signal index and absolute signal intensity was carried out as discussed in FIG. 2 using computer algorithms.

Stable Isotope Labeling with Amino Acids in Culture (SILAC)
Isotopically labelled amino acids were purchased from Cambridge Isotope Laboratories, Inc. (USA): L-Lysine (13C6, 15N2)—cat. no. CNLM-291-H-PK; L-Lysine (1306)—cat. no. CLM-2247-H-PK; L-Arginine (D7, 15N4)—cat. no. DNLM-7543-PK; L-Arginine (1306)—cat. no. CLM-2265-H-PK. Jurkat and A431 cells were labelled with heavy amino acids (Lysine 13C6, 15N2; Arg 15N4, D7). RT4 and HeLa cells were labelled with medium amino acids (Lysine 13C6; Arg 13C6). U2-OS and MCF7 were labelled with light amino acids. First, the cells were adapted to dialyzed FBS. All cell lines were grown in RPMI 1640 (without lysine, arginine and glycine) supplemented with 10% dialyzed FBS (Sigma, cat. no. F0392-100 ML), penicillin/streptomycin, 1.1494253 mM light L-arginine, 0.2739726 mM light L-Lysine hydrochloride and 2.0547945 mM light L-glutamine. The cells were passaged at least 5 times to assess the effect of dialyzed FBS on growth and morphology. During this stage the cells were maintained in standard T25 flasks. After adaptation, the cell lines were grown in RPMI 1640 medium (no lysine, arginine, glycine) supplemented with 10% dialyzed FBS, penicillin/streptomycin and either heavy, medium or light amino acids. The cells were grown for at least 5 population doublings to ensure maximal incorporation of the labels.

### Example 1—Antibody Specificity Analysis Using Parallel Mass Spectrometry and Antibody Array

### Materials and Methods

Antibody specificity analysis was carried out in accordance with FIG. 1. The methods for carrying out the fractionation and Western Microsphere Affinity Proteomics (WMAP) analysis are discussed above and are detailed in International patent publication WO 2009/080370.

Cells from three different cell types (RT4 cells, U2OS cells and HeLa cells), or alternatively from primary CD4 T cells that are either unstimulated, stimulated with the mitogen concanavalin A for 24 hours or stimulated with concanavalin A for 48 hours, were lysed, and soluble proteins in cell lysates were denatured and were labelled with biotin as described above. The proteins were then further denatured and separated by gel electrophoresis using a GELFREE® 8100 instrument as described above. A liquid handling robot was used for precise transfer of liquid fraction aliquots from the master plate to two replicate plates.

The wells of one of these two replicate plates was supplemented with bead-based antibody arrays as described above and analysed using flow cytometry.

The other plate was processed for analysis of peptides by mass spectrometry as described above.

The approach described above yields two sets of numerical data. Data was analysed as described above.

### Results and Discussion

As shown in FIG. 2, the use of MS in parallel with WMAP is able to distinguish between antibodies that bind specifically (e.g. with high specificity) to Akt1 (FIG. 2A) from antibodies that do not (or bind with lower specificity) (FIG. 2B). The most specific antibodies show good overlap between the WMAP antibody array data (solid lines) and the MS data (dotted lines). This method is also able to provide information on antibody sensitivity, for example the antibodies shown in FIGS. 2C and 2D are less sensitive than the antibody of FIG. 2A, based on the maximum (or absolute) MFI.

The ability of the method to distinguish specific antibodies from non-specific antibodies is again shown with respect to anti-RBL2 antibodies (FIGS. 3A and 3B) and with respect to anti-beta actin antibodies (FIGS. 3C to 3E). Since the MS data represent the gold standard, one can safely conclude that the antibodies in charts B and D are specific (good overlap of WMAP and MS data) while those in A and C are not (little overlap of WMAP and MS data), i.e. are cross-reactive or non-specific antibodies. Since flow cytometry has a high dynamic range for fluorescence detection, one can also conclude that the antibody in D is more sensitive than the one in E, based on the maximum (or absolute) MFI.

Through the use of heat maps as shown in FIG. 4, the parallel MS and WMAP analysis can be carried out with respect to a large number of antibodies, and so antibody screening can straightforwardly be carried out. The level of precision seen in the heat maps is highly unexpected for a relatively crude fractionation of a total cell lysate.

### Example 2—Elution of Proteins from Anti-CD3E and Anti-CD247 Antibodies

### Materials and Methods

CD4+ T cells were lysed and labelled as described above for native proteins. Separation was carried out with respect to four subcellular locations (i.e. subcellular fractionation), namely (1) cytosol, (2) organelles, (3) nucleus and cytoskeleton and (4) membrane locations, using established methods, and with respect to size using size exclusion chromatography. The fractions were then separated and analysed with antibody arrays and flow cytometry as described above.

The flow cytometry data was processed through R script analysis in order to determine the fraction with the highest levels of membrane-associated targets for anti-CD3e and anti-CD247 antibodies (shown by the longer arrows in FIG. 5A). An aliquot of that fraction was taken from the master plate and captured with an anti-CD3e antibody or with an anti-CD247 antibody attached to particles. After two washes in ice-cold PBT, the proteins bound to the antibodies were eluted with a 30 minute incubation in 1% Tween® 20 in PBS at 22° C. under constant agitation. The eluent was transferred to further antibody array (where antibodies were attached to colour-coded particles) as described above and analysed using flow cytometry.

A further elution was carried out in order elute proteins still bound to the antibodies in a solution of 0.1 SDS at 95° C. The eluent was transferred to further antibody array as described above and analysed using flow cytometry.

### Results and Discussion

The results are shown in FIG. 5. While the arrays contain 576 antibodies to a wide range of proteins, anti-CD3e and anti-CD247 antibodies pull down components of the T cell receptor complex (CD3e, CD247, Zap70, Trat1 and LCK). In both cases, native mild elution allows detection with multiple different antibodies to CD3e. Some of these antibodies do not detect protein after denatured elution (heat+SDS), and they are therefore likely to bind to conformation-dependent epitopes that are lost during denaturing conditions. The results show that two antibodies to different components of a complex pull down similar proteins. This allows direct assessment of the specificity of individual antibodies.

This example shows not only that surprisingly mild elution conditions can be used in combination with the WMAP analysis but also that such mild elution conditions advantageously allow for the analysis of conformation-dependent epitopes and the identification of antibodies that bind to such epitopes.

### Example 3—Array Based Antibody Validation

### Materials and Methods

### Cell Lines and Culture Conditions:

The human Urinary Bladder Papilloma cell line RT4 (cat. no. 300326) and the Human Osteosarcoma cell line U2-OS (cat. no. 300364) were purchased from CLS Cell Lines Service (Germany). The acute T-cell leukemia cell line Jurkat (clone E6-1, cat. no. ATCC TIB-152), the epidermoid carcinoma epithelial cell line A-431 (cat. no. ATCC CRL-1555), the mammary gland adenocarcinoma cell line MCF7 (cat. no. ATCC HTB-22) were purchased from ATCC. The cervical adenocarcinoma cell line HeLa was a kind gift from M.S. Rødland (Oslo University Hospital, Oslo, Norway). The cell lines used in the study were authenticated by STR analysis via an external service provider (Identicell, Aarhus, Denmark). HeLa, RT4, A431, U2-OS, MCF7 and Jurkat cells were grown in RPMI 1640 medium supplemented with 10% FBS and penicillin/streptomycin. The cells were cultivated in a humidified atmosphere with 5% CO2 at 37° C. The cells were maintained in standard T75 flasks and expanded in T175 flasks prior to harvest.

Stable Isotope Labeling with Amino Acids in Culture (SILAC):

Isotopically labelled amino acids were purchased from Cambridge Isotope Laboratories, Inc. (USA): L-Lysine (13C6, 15N2)—cat. no. CNLM-291-H-PK; L-Lysine (13C6)—cat. no. CLM-2247-H-PK; L-Arginine (D7, 15N4)—cat. no. DNLM-7543-PK; L-Arginine (13C6)—cat. no. CLM-2265-H-PK. Jurkat and A431 cells were labelled with heavy amino acids (Lysine 13C6, 15N2; Arg 15N4, D7). RT4 and HeLa cells were labelled with medium amino acids (Lysine 13C6; Arg 13C6). U2-OS and MCF7 were labelled with light amino acids. First, the cells were adapted to dialyzed FBS. All cell lines were grown in RPMI 1640 (without lysine, arginine and glycine) supplemented with 10% dialyzed FBS (Sigma, cat. no. F0392-100 ML), penicillin/streptomycin, 1.1494253 mM light L-arginine, 0.2739726 mM light L-Lysine hydrochloride and 2.0547945 mM light L-glutamine. The cells were passaged at least 5 times to assess the effect of dialyzed FBS on growth and morphology. During this stage the cells were maintained in standard T25 flasks. After adaptation, the cell lines were grown in RPMI 1640 medium (no lysine, arginine, glycine) supplemented with 10% dialyzed FBS, penicillin/streptomycin and either heavy, medium or light amino acids. The cells were grown for at least 5 population doublings to ensure maximal incorporation of the labels.

### Cell Lysis:

Adherent cells (A431, HeLa, MCF7, U2-OS, RT4) were harvested by trypsinization, followed by two washes in PBS (Sigma, cat. no. D8537). Suspension cells (Jurkat) were washed twice in PBS before lysis. The pellets were then re-suspended in SDS lysis buffer (15 mM NaCl, 30 mM HEPES pH 7.4, 1 mM EDTA, 2 mM MgCl2, 0.3% SDS) supplemented with protease inhibitor cocktail (Sigma, cat. no. P8340-5 ML), 1 mM TCEP, 1 mM PMSF, 1 mM NaF, 1 mM Na3VO4 and incubated for 10 min at 95° C. Buffer volume used was equal to 15 cell pellet volumes. The lysates were cooled on ice to room temperature and 250 units of benzonase (Semba Biosciences, cat. no. R1006E) was added. The samples were incubated for 30 min at 37° C., centrifuged at 14000 g for 5 min, aliquoted and stored at −70° C. Protein concentration was measured using DirectDetect assay free cards using the Direct Detect instrument (MerckMillipore)

### Biotinylation of Sample Proteins:

Protein (300 μg) from each cell type was supplemented with sulfo-NHS-LC-Biotin and Biotin-PEG2-maleimide (both at 0.5 mg/ml, www.proteochem.com). The samples were incubated 30 min on ice. Free biotin and salts were removed by buffer exchange using 10 kDa Amicon filters (MerckMillipore, cat. no. UFC501096). The sample was added to the filter and centrifuged at 14000×g for 10 min, and the flow through was discarded. Deionized water (450 μl) was added on top of the filter and centrifugation was repeated. The procedure was repeated four times. After the last step, 50 μl of water was added to the filter, which was then inverted and placed in a clean collection tube. The filters were centrifuged at 2000×g for 2 min. Protein concentration was determined using the DirectDetect instrument (MerckMillipore).

### Preparative Gel Electrophoresis by Gelfree 8100:

A Gelfree 8100 instrument (Expedeon, UK) was used to obtain liquid fractions with size-separated proteins using installed programs for gels with three different separation ranges: Tris-Acetate 5% (80-300 kDa), TA 8% (35-90 kDa), 10% (15-70 kDa). For each separation, a total of 150 μg protein was supplemented with SDS-sample buffer for Gelfree separation (Expedeon UK). Fractions (150 μl) were harvested at 12 time points as recommended by the manufacturer and transferred to a 96 well plate. The fractions were stored at −70° C. until use.

### Solid-Phase-Aided Sample Preparation (Solid-PhASP) of Peptides for Mass Spectrometry (MS):

50 μl of each fraction from the Gelfree separation was transferred to a 96 well PCR plate pre-filled with 100 μl PBS (Axygen cat no 732-0662). Five microliters of a 50% streptavidin sepharose slurry was added (http://www.gelifesciences.com/). Prior to use, the streptavidin beads were treated with the 50μγ/ml of Bissulfosuccinimidyl suberate (BS3) for 15 min at 22° C. crosslink the streptavidin and thereby minimize release of streptavidin-derived peptides during on-bead trypsin digestion. Microwell plates with sample proteins and streptavidin beads were sealed with caps and rotated for 30 min at 22° C. to immobilize biotinylated proteins. The sepharose beads were next washed twice in PBS with 1% DDM to remove detergents, twice with deionized water and resuspended in 100 μl ammonium carbonate buffer. At this point beads with separated proteins from three SILAC-labelled cell types were mixed to allow multiplexed MS. Trypsin (1 μg) was added to each well, and the plate was incubated with constant shaking overnight at 37° C. The streptavidin beads were pelleted by centrifugation and the supernatant containing peptides was transferred to a Sep-Pak tC18 μElution filter plate (Waters, cat. no. 186002318). The resin was pre-activated using 100 μl acetonitrile (Sigma), followed by equilibration with 200 μl of 0.1% formic acid in water. Peptides were passed through the filter plate using a vacuum manifold. The resin was then washed twice with 200 μl of 0.1% formic acid in water. The peptides were eluted in two subsequent rounds, each time using 80 μl 80% acetonitrile with 0.1% formic acid in water. The samples were dried using a Concentrator Plus vacuum concentrator (Eppendorf) and the volume was adjusted to 12 μl using 0.1% formic acid in water. The samples were stored at −20° C. until use.

### Mass Spectrometry:

Peptides were analyzed on QExactive plus Orbitrap mass spectrometer coupled to Easy-nLC1000 liquid chromatographer (both ThermoFisher Scientific). LC was equipped with a 50 cm PepMap RSLCC18 column with a diameter of 75 μm (ThermoFisher Scientific, cat. no. ES803). Water with 0.1% formic acid was used as solvent A and acetonitrile with 0.1% formic acid was used as solvent B. The gradient was as follows: 2% B to 7% B in 5 min; 7% B to 30% B in 55 min; 30% B to 90% B in 2 min; 90% B for 20 min. Solvent flow was set to 300 nl/min and column temperature was kept at 60° C. The mass spectrometer was operated in the data-dependent mode to automatically switch between MS and MS/MS acquisition. Survey full scan MS spectra (from m/z 400 to 1,200) were acquired in the Orbitrap with resolution R=70,000 at m/z 200 (after accumulation to a target of 3,000,000 ions in the quadruple). The method used allowed sequential isolation of the most intense multiply-charged ions, up to ten, depending on signal intensity, for fragmentation on the HCD cell using high-energy collision dissociation at a target value of 100,000 charges or maximum acquisition time of 100 ms. MS/MS scans were collected at 17,500 resolution at the Orbitrap cell. Target ions already selected for MS/MS were dynamically excluded for 30 seconds. General mass spectrometry conditions were: electrospray voltage 2.1 kV; no sheath and auxiliary gas flow, heated capillary temperature of 250° C., normalized HCD collision energy 25%. Ion selection threshold was set to 5e4 counts. Isolation width of 3.0 Da was used.

### Analysis of MS Data:

MS raw files were submitted to MaxQuant software version 1.5.2.8 for protein identification. Parameters were set as follows: no fixed modification; protein N-acetylation and methionine oxidation as variable modifications. When applicable, the following SILAC labels were selected: Lys8; Arg11; Lys6; Arg6. First search error window of 20 ppm and mains search error of 6 ppm. Trypsin without proline restriction enzyme option was used, with two allowed miscleavages. Minimal unique peptides were set to 1, and FDR allowed was 0.01 (1%) for peptide and protein identification. The reviewed Uniprot human database was used (retrieved June 2015). Generation of reversed sequences was selected to assign FDR rates.

### Microsphere-Based Antibody Arrays.

Microspheres with up to 500 fluorescent bar codes are commercially available from Luminex corporation. The procedure for production of the in-house arrays used here has been described in detail previously (Wu et al., Molecular and Cellular Proteomics: MCP 8: 245-257, 2009; Slaastad et al., Proteomics 11, 4578-4582, 2011). Briefly, amine functionalized polymethyl-metha-acrylate (PMMA) microspheres (Bangs Laboratories, IN, USA) first reacted with the hetero-bifunctional crosslinker succinimidyl 3-(2-pyridyldithio)propionate (SPDP, 50 μg/ml, Sigma) and reduced with 5 mM TCEP (Sigma) to obtain thiol-functionalized beads. The thiol groups were first used as binding sites for maleimide-derivatized Protein G (ProSpec-Tany TechnoGene Ltd, IL). Remaining thiols were used to bind serially diluted solutions of malemide-derivatives of fluorescent dyes: Alexa-750 (three levels), Alexa-488 (six levels), Alexa-647 (six levels), Pacific Orange (four levels) and Pacific Blue (four levels). Antibodies from rabbit and goat were coupled directly to protein-G beads. For binding of mouse antibodies, the beads were first coupled with goat antibodies to mouse IgG subclasses (Jackson lmmunoresearch). Bar-coded microspheres were kept separate in 384 well plates until completion of the antibody coupling step. The beads were next mixed suspended in PBS Casein Block buffer (Thermo Fisher) and stored at −70° C. until use.

### Antibody Array Analysis.

Aliquots (15 μl) of the fractions obtained by GelFree separation (see above) were added to a microwell plate pre-filled with 150 μl PBT. The samples were next supplemented with 10 μl of a solution containing bead-based antibody arrays suspended in PBS casein block buffer supplemented with immunoglobulins (20 μg/ml) from human, mouse and goat IgG. The plate was sealed with plastic film and rotated overnight at 4-8° C. The plate was next centrifuged at 1000×g to pellet the beads. The supernatant containing unbound protein was harvested and stored frozen. The beads were next washed twice in PBT and labelled with R-Phycoerythrin-conjugated streptavidin (10 μg/ml in PBS with 0.1% bovine serum albumin, Jackson Immunoresearch). Following two washes with PBT, the beads were resuspended in PBS with 0.1% bovine serum albumin and analyzed by flow cytometry.

### Flow Cytometry.

Microsphere-based antibody arrays were analyzed using an Attune flow cytometer (Thermo) equipped with a 96 plate sample loader and four lasers: 405 nm (Pacific Blue, Pacific Orange), 488 nm (Alexa-488), 567 nm (R-Phycoerythrin) and 633 nm (Alexa-647, Cy7). The emission filters were standard for the instrument, except for the use of a 520 nm band bass filter for detection of Pacific Orange.

### Analysis of Flow Cytometric Data.

Flow cytometry data were processed using a freely available R-application dedicated for analysis of MAP data (Stuchly et al., 2012, supra). The application identifies microsphere subsets on basis of their color codes and exports values for median R-Phycoerythrin fluorescence for each subset.

### Statistics:

The MS and flow cytometry procedures described above yield two sets of numerical data which can be correlated. All correlations reported are Pearson correlations for linear data. To assess the frequency of random correlations in MAP-MS and transcriptomics datasets, the proteins/mRNA identifiers were first sorted according to predicted mass and then in alphabetical order. We next assessed correlations between data in neighboring rows. Correlations between series of six values corresponding to relative abundance of proteins or mRNA were assessed for MS and transcriptomics data. For MAP and MS data we also assessed the overall correlation between all data points in fractions 3-12 in all samples. The results in FIG. 9B show that the frequencies of random correlations of 0.9 are around 5%, which corresponds to statistical significance (p<0.05). The rationale for choosing a lower cut-off for validation is that the average correlation between results in the two MS datasets was 0.6, and fewer than 40% of the correlations were higher than 0.9 (data not shown). The same was true for correlations between the two transcriptomics datasets (data not shown). We re-analyzed data from biological replicates in the MaxQB database and obtained similar results (Geiger et al., Molecular and Cellular Proteomics: MCP 11, M111 014050, 2012). Thus, the precision that can be obtained with orthogonal data is limited by the reproducibility of the methods used to generate reference data. However, a significance of 0.05-0.15 for discrimination between proteins with the same mass is clearly better than the current industry standard, which is a band near or at a predicted position and no sample named as positive and negative control.

### Results and Discussion

The method described in this Example is analogous to a multiplexed Western Blot (WB) with MS data as a direct reference to assess specificity (FIG. 1). The first steps are the same as for standard WB (materials and methods). Thus, proteins from six human cell lines were heated in the presence of sodium dodecyl sulphate (SDS) and separated by polyacrylamide gel electrophoresis (PAGE). However, to facilitate multiplexed analysis with antibody arrays, we labelled the sample proteins with biotin and used the Gelfree® 8100 instrument (Expedeon, UK) for preparative PAGE. The instrument yielded 12 liquid fractions with size-separated, biotinylated proteins from each sample (FIG. 1). An aliquot of each fraction was analysed with microsphere-based antibody arrays and flow cytometry (microsphere affinity proteomics, MAP, Wu et al., 2009, supra). A second aliquot was processed with a new semi-automated method (Solid-PhASP) to obtain peptides for MS (FIG. 1 and FIG. 9A). Analysis by MAP resolved antibody targets as peaks of reactivity across the fractions, and PAGE-MS data for the intended targets served as reference to identify peaks that correspond to specific binding (FIG. 1, numerical data not shown). 2412 antibodies were used.

Text files with data from two PAGE-MAP/MS experiments (data not shown) were used as input in computerized antibody validation (CAVA, supplementary software, supplementary protocol). The algorithm focusses on fractions 3-12, which contain the best resolved proteins. The first steps in the validation process are assessment of signal to noise ratio (signal index) and peak position (or core index) (FIGS. 6A and 6B, FIG. 8). The threshold for signal to noise (signal index) was set to a four-fold difference between the strongest and the median MAP signal measured across all samples. CAVA next determines if the tallest MAP peak overlaps with the MS peak for the intended target in the same sample. A deviation of one fraction is accepted for this core index (or peak position) (FIG. 8).

The result of the first two steps was visualized as heatmaps formatted as “digital WBs” (FIGS. 6A and 6B). Thus, the largest protein appears on top, and the remainder are organized in descending order according to predicted mass to mimic their positions on a standard WB. Since protein mass increases along the y-axis as well as with fraction number (x-axis), the expected pattern is a continuum of “bands” from the lower left to the upper right in each map. The MS data in the “digital WBs” showed the expected pattern (FIGS. 6A and 6B). The same was true for targets of antibodies that passed thresholds for sensitivity and peak position (FIG. 6A). By contrast, the reactivity pattern of antibodies that failed to meet these criteria was dominated by background signal (FIG. 6B).

Thus, through the use of heatmaps as shown in FIGS. 6A and 6B, one can visualize the results of a computer algorithm used to process results from parallel analysis of fractionated proteins by MAP and MS to identify specific antibodies. The maps in FIG. 6A shows antibody reactivity patterns (left half) that closely resemble the MS data for the corresponding targets (right half). The antibodies were identified on the basis of computerized assessment of the signal index (SI) as having an SI of four or more. The algorithm also determined that the maximal antibody signal was measured in the same fraction as the maximal MS signal or in one of the immediate neighboring fractions. The antibodies in FIG. 6B failed to meet these criteria, and the heatmap shows a clear difference between their reactivity patterns and the MS data.

The heatmaps shown in FIG. 7 serve to further illustrate how a computer algorithm can be used to process data from parallel analysis of fractionated proteins with antibody arrays and MS. In these heatmaps, the 12 data points from analysis of fractionated samples are compressed to a single value corresponding to the wide index (sum of signal measured in five fractions centered around the maximum). The wide index serves as proxy for protein abundance. With this analysis, the signature of the protein is the relative abundance in different cell lines (J Jurkat, U U2OS, H HeLa, A A431, R RT4, M MCF7). The computer algorithm identified 302 antibodies with reactivity patterns that had correlations of 0.9 or better with MS data. This is observed as similarity between the heatmaps for MAP and MS data in the upper heatmap. The heatmaps to the right show that a similar pattern was observed for differential mRNA expression. The mRNA data were retrieved from two published datasets and therefore serve as an independent reference (Uhlen et al Science 2016, Klijn C. et al Nat Biotechnol 33, 306-312 (2015)). The lower heatmap shows results obtained with antibodies that failed to meet critera for correlation between MAP and MS data. This is observed as a difference between the heatmaps shown for antibody reactivity and MS and mRNA data.

A key feature of the present invention is that the analysis of relative protein abundance in a series of fractions yields a chromatogram that serves as a signature for the protein of interest. Antibody validation is based on correlation of chromatograms obtained when the fractions are analyzed with antibody arrays and MS, respectively. We provide an example to illustrate how one can use MS data to determine the level of correlation required to obtain statistical significance.

The heatmap in FIG. 9A and the line chart in FIG. 9B serve to illustrate how results from shotgun MS analysis can be used to assess the significance of correlations. The heatmap in FIG. 9 visualizes the entire MS dataset obtained by measuring fractions from six cell lines separated by three gels with different separation range (5%, 8% or 10% acrylamide). The proteins were processed and analyzed by shotgun MS analysis as described in FIG. 1. The proteins in the dataset were sorted according to the type of gel used for separation and then in descending order according to predicted mass. Since protein mass also increases with fraction number (x-axis), the expected pattern is a continuum of “bands/pixels” along the diagonal from bottom left to top right in each map. To assess random correlations, the data in each row were correlated to those in the row below. The line chart in FIG. 9B shows the frequency/significance (y-axis) of random correlations indicated on the x-axis, and the horizonal line indicates a frequency of 0.05, which is often used as a threshold for significance in statistics. The two lines correspond to results obtained in two separate experiments. Thus, one can readily observe that a correlation of 0.8-0.9 is statistically significant.

The dot plots in FIG. 10 serve to illustrate the added value of analyzing fractionated samples as compared to measuring protein abundance. The left dot plot show overall correlations in experiment 1 plotted against those in experiment 2 (i.e. correlation all datapoints obtained by paired analysis of 12 fractions by MAP and MS, respectively). The squared R value was 0.7 which indicates that highly similar correlations were observed in the two experiments. The dot plot to the right shows corresponding results for measurements of the wide index (i.e. sum of five fractions centered around the maximum as proxy for relative protein abundance) The squared R value was 0.25. The results show that correlations for dataseries consisting of all data points are more reproducible (i.e. higher correlation between the two experiments) than what is achieved by measuring protein abundance. This result is surprising and underscores the added value of analyzing fractionated samples.

### Example 4—Mass Spectrometry Analysis of Monomeric Proteins Captured from Enriched Page Fractions

### Materials and Methods

Stable Isotope Labeling with Amino Acids in Culture (SILAC):

Human T cell acute leukemia cells (Jurkat) were adapted to culture in medium with dialyzed fetal bovine serum (FBS) by culture in RPMI 1640 (without lysine, arginine and glycine) supplemented with 10% dialyzed FBS (Sigma, cat. no. F0392-100 ML), penicillin/streptomycin, 1.1494253 mM light L-arginine, 0.2739726 mM light L-Lysine hydrochloride and 2.0547945 mM light L-glutamine. The cells were passaged at least 5 times to assess the effect of dialyzed FBS on growth and morphology. After adaptation, the cell lines were grown in RPMI 1640 medium (no lysine, arginine, glycine) supplemented with 10% dialyzed FBS, penicillin/streptomycin and heavy isotope acids (Lysine 13C6, 15N2; Arg 15N4, D7). The cells were grown for at least 5 population doublings to ensure maximal incorporation of the labels.

The methods for preparation of cell lysates, labeling of proteins with biotin, separation by Gelfree 8100 and analysis by MAP and MS are described above.

### Immunoprecipitation and Mass Spectrometry:

Indicated amounts of biotinylated proteins from Gelfree® 8100 fractions was diluted in 1 ml PBS with with 0.1% casein (Thermo Fisher, cat no. 37528). Polymer beads coupled covalently with Protein A/G (Prospec, IL) and then with indicated antibodies were added (1 ul 10% solids). The mixture was incubated overnight at 4-8° C. with constant shaking. The beads were pelleted by centrifugation and washed twice in PBS with 0.1% dodecyl maltoside. The beads were next resuspended in 100 μl ammonium carbonate buffer, and 100 ng trypsin (Promega) was added. After 15 min incubation at 21° C., the beads were pelleted and the supernatant was harvested. Peptides were processed for mass spectrometry as described above.

### Results and Discussion:

The line chart in FIG. 11 shows signal intensity (y-axis) for beta-actin measured by mass spectrometry (dashed line) and antibody array analysis (solid line, anti-beta-actin antibody GTX629630, GeneTex, USA), plotted against fraction number (Gelfree® 8100 fractionation, 10% gel). The maximum signal was observed in fraction 8, and the trace for the target of the antibody closely resembles the MS signal for beta actin. The antibody is therefore readily identified as a good candidate for more expensive validation by immunoprecipitation and mass spectrometry (IP-MS).

One microliter of fraction (8) with an estimated content of as little as 100 ng protein was used as source for immunoprecipitation with anti-beta actin antibody. The immune-precipitate was processed for MS analysis as described above. The bar graph in the middle shows MS signal intensity for indicated proteins with SILAC labeling (log scale), while the graph to the right shows signal for proteins without SILAC label.

The results show that only five proteins in the immunoprecipitate contained the SILAC label, and more than 90% of the total MS signal for SILAC-labelled proteins corresponded to the antibody target (beta-actin, ACTB). A large number of additional proteins were observed (right bar chart). However, these did not contain the SILAC label and therefore represent sample contamination. The signals from contaminating proteins were up to ten-fold stronger than that observed with SILAC-labelled beta-actin. While some of the contaminating proteins represent keratins that are known to be common contaminants, many are broadly expressed cellular proteins, and the list also contains non-keratin proteins. Collectively, the results obtained by paired antibody array and MS analysis and the downstream analysis by IP-MS provide definitive evidence that the antibody to beta-actin is more than 90% specific for the intended target.

The solid line in the line chart in FIG. 12 shows signal intensity for anti-RELA (y-axis, log scale) plotted against Gelfree fraction number. The dashed line shows MS signal for RELA. The trace obtained with the antibody closely resembles the MS signal for the intended target. The antibody is therefore clearly a good candidate for definitive validation by IP-MS. The bar chart in the middle shows MS signal for SILAC-labelled proteins. Rel A was detected in immunoprecipitates from 1 ul and 10 ul Gelfree fraction, corresponding to an estimated 10 ug and 1 ug of protein, and the protein, and intended antibody target constituted more than 90% of the total MS signal for SILAC-labelled proteins. The bar chart to the right shows presence of a large number of proteins with higher MS signal intensity than that measured for RELA. However, these proteins did not contain the SILAC label and therefore represent contamination. Collectively, the results obtained by paired antibody array and MS analysis and the downstream analysis by IP-MS provide definitive evidence that the antibody to RELA is more than 90% specific for the intended target.

Established protocols for IP-MS describe the use of 0.5-5 mg of sample protein (Marcon, E. et al., Nat Methods, 12, 725-731 (2015); Malovannaya A. et al, Cell, 145, 787-799 (2011). Here, we used as little as 1 ug to detect RELA and 100 ng for detection of beta-actin. Thus, the sensitivity of method described in the present invention is three orders of magnitude higher. Moreover, immunoprecipitates obtained using established protocols contain an average of at least 200 proteins as compared to five proteins or less with the method described here (Marcon, E. et al., Nat Methods, 12, 725-731 (2015). The most comprehensive study to date concluded that the precision of specificity assessment in IP-MS is limited to showing that the intended target is among the top-three most abundant proteins in the immunoprecipitate (Marcon, E. et al., Nat Methods, 12, 725-731 (2015). A second large study concluded that “our analysis provides indication, but NOT a conclusive proof for identities of secondary (cross-reacting) antigens.” Malovannaya A. et al, Cell, 145, 787-799 (2011), supplementary Table 1). The results obtained with the method described in the present invention are therefore surprising and clearly more definitive.

We conclude that paired analysis of fractionated proteins with antibody arrays and MS is helpful to select antibodies that are likely to be specific and therefore worth the investment of more expensive and definitive downstream analysis by IP-MS. It is also clear that this method will be useful to identify the targets of antibodies that cross-react. In paired array and MS analysis of fractions, one would identify an antibody reactivity peak that does not overlap with the MS signal. The antibody can then be used to immunoprecipitate the target from the enriched fraction for identification by IP-MS. Finally, some antibodies may show a reactivity peak when shotgun MS does not show a signal for the intended target. A negative MS signal is not definitive evidence for lack of protein expression. IP-MS is more sensitive than shotgun MS. One can therefore identify targets of antibodies to low abundance proteins that are not detected by shotgun MS.

1. A method of analysing a mixture of polypeptides comprising the steps of:
(i) separating the polypeptides in the mixture into a plurality of fractions;
(ii) contacting a first aliquot of two or more of the fractions with a plurality of different binding agents attached to one or more solid supports and detecting the binding of the polypeptides to the binding agents in each fraction;
(iii) assessing the amino acid composition of the polypeptides in a second aliquot of said fractions by mass spectrometry; and
(iv) correlating the binding results detected in step (ii) and the mass spectrometry results from step (iii) to assess the specificity of the binding agents for a polypeptide of interest.

2. The method of claim 1 further comprising the steps of:
(v) determining one or more fractions which are enriched for a particular polypeptide of interest;
(vi) contacting the one or more fractions with a binding agent to said polypeptide of interest attached to one or more solid supports;
(vii) disrupting the binding agents of step (vi) from the associated polypeptides; and
(viii) contacting the released polypeptides with a plurality of binding agents attached to one or more solid supports and detecting the binding of the polypeptides to the binding agents.

3. The method of claim 1 further comprising the steps of:
(v) determining one or more fractions which are enriched for a particular polypeptide of interest;
(vi) contacting the one or more fractions with a binding agent to the polypeptide of interest attached to one or more solid supports;
(vii) disrupting the binding agents of step (vi) from the associated polypeptides;
(viii) contacting the released polypeptides with a soluble binding agent that binds specifically to a first epitope on the polypeptide of interest; and
(ix) contacting the polypeptides bound to said soluble binding agent with a plurality of binding agents attached to one or more solid supports and detecting the binding of the binding agents attached to the one or more solid supports to the polypeptides of interest.

4. The method of claim 1 further comprising the steps of:
(v) determining one or more fractions which are enriched for a particular polypeptide of interest;
(vi) contacting the one or more fractions with a binding agent to the polypeptide of interest attached to one or more solid supports;
(vii) disrupting the binding agents of step (vi) from the associated polypeptides; and
(viii) assessing the amino acid composition of the released polypeptides by mass spectrometry (MS).

5. The method of claim 4, wherein the disruption step (vii) is carried out by treating the solid support with a proteolytic enzyme to generate peptides that can be analysed by MS.

6. The method of claim 4, wherein the MS analysis is multiplexed using addressable bar codes, preferably where the addressable bar code is a stable isotope or is a physical parameter specific for proteins in a certain fraction.

7. The method of claim 1, wherein the separation step (i) is comprised of the following steps:
(i.a) separation of polypeptides in the mixture into a plurality of fractions;
(i.b) contacting a first aliquot of two or more of the fractions with a plurality of different binding agents attached to one or more solid supports and detecting the binding of the polypeptides to the binding agents in each fraction;
(i.c) determining one or more fractions which are enriched for a particular polypeptide of interest;
(i.d) separating the enriched fractions into a plurality of fractions.

8. The method of claim 7 where the steps in claim 7 are repeated one or more times.

9. The method of claim 2 wherein the binding agents of step (vii) are disrupted from the associated polypeptides using successive solutions with increasing stringency.

10. The method of claim 2 wherein the disruption of step (vii) is carried out using a nonionic surfactant, preferably a polysorbate-type non-ionic surfactant, more preferably polysorbate 20.

11. The method of claim 10 wherein a further or second disruption is carried out at step (vii) using an anionic surfactant, preferably an organosulphate surfactant, more preferably sodium dodecyl sulphate.

12. The method of claim 1 further comprising carrying out steps (i) to (iv) in respect of one or more further mixtures of polypeptides, preferably one or more further cell types.

13. The method of claim 1 wherein step (i) comprises separating the polypeptides on the basis of one or more physical parameters and/or subcellular locations and/or mixtures of polypeptides.

14. The method of claim 13 wherein the one or more physical parameters are selected from the list consisting of differential mass, acidity, basicity, charge, hydrophobicity and binding to different affinity ligands.

15. The method of claim 1 wherein step (i) is carried out using one or more techniques selected from the list consisting of gel electrophoresis, size exclusion chromatography, liquid chromatography, dialysis, filtration, ion exchange separation and iso-electric focusing.

16. (canceled)

17. The method of claim 1 wherein the binding agent of step (ii) is selected from the list consisting of antibodies or antigen-binding fragments thereof, aptamers or other nucleic acid based binding agents, affibodies, polypeptides, peptides, oligonucleotides, T-cell receptors, MHC molecules and mixtures thereof.

18. The method of claim 2 wherein the binding agent of any one of steps (vi) or (viii) is selected from the list consisting of antibodies or antigen-binding fragments thereof, aptamers or other nucleic acid based binding agents, affibodies, polypeptides, peptides, oligonucleotides, T-cell receptors, MHC molecules and mixtures thereof.

19. The method of claim 1 wherein the step (i) comprises separating the polypeptides in the mixture into at least four fractions, preferably at least twelve fractions, more preferably at least twenty four fractions, more preferably at least forty eight fractions, more preferably at least ninety six fractions, more preferably at least 200 fractions.

20. The method of claim 1 wherein the binding agents attached to one or more solid supports are attached in an array on the surface of one or more planar substrates and/or a planar substrate comprising three-dimensional surface structures.

21. The method of claim 1 wherein the binding agents are attached to a plurality of particles, each particle having attached thereon multiple copies of the same binding agent.

22. The method of claim 21 wherein a first set of particles having attached thereon multiple copies of the same binding agent have a different detectable feature from a further set of particles having multiple copies of a binding agent that are different to those attached to the first set of particles.

23. The method of claim 22 wherein the detectable feature is based on fluorescence, isotopes, preferably radioactive isotopes or non-radioactive (stable) isotopes, luminescence, size or acoustic properties.

24. The method of claim 23 wherein the detectable feature is in the form of at least one type of dye molecule attached to the particle, preferably at least three types of dye molecules attached to the particle.

25-27. (canceled)

28. The method of claim 1 further comprising attaching at least one label to the mixture of polypeptides or the one or more further mixtures of polypeptides.

29. The method of claim 28 wherein the step of attaching the label or labels to the mixture of polypeptides or the one or more further mixtures of polypeptides is carried out prior to step (i) or after step (i).

30. The method of claim 28 wherein a different label is attached to the mixture of polypeptides or the one or more further mixtures of polypeptides of each fraction.

31. The method of claim 28 wherein the label is attached to the polypeptides via a peptide, a polypeptide, an oligonucleotide, or an enzyme substrate.

32. The method of claim 28 wherein the or each label is selected from the list consisting of a hapten, a fluorescent dye, a luminescent dye, a radioactive isotope, a non-radioactive isotope and a mixture thereof.

33. The method of claim 32 wherein the hapten is biotin or digoxigenin.

34. The method of claim 1 wherein step (iv) is carried out by determining the correlation between the binding results of step (ii) in a chosen set of fractions and the MS results of step (iii) in the same fractions; or wherein step (iv) is carried out by measuring the overlap between the binding results of step (ii) and the MS results of step (iii).

35. The method of claim 1, wherein the binding results of step (ii) in a chosen set of fractions and the MS results of step (iii) in the same fractions are in the form of sets of numerical data which are then correlated in step (iv).

36. The method of claim 1, wherein a correlation which is statistically significant with a probability of p<0.20, p<0.15, p<0.10 or p<0.05 is indicative of a binding agent that is specific for the polypeptide of interest.

37. The method of claim 1 wherein step (iv) comprises processing either the binding results of step (ii) and/or the MS results of step (iii) in order to make direct comparisons between the binding results and the MS results.

38. The method of claim 37 wherein the processing comprises (a) upscaling or downscaling the binding results of step (ii) so that they can be compared against the MS results of step (iii); (b) upscaling or downscaling the MS results of step (iii) so that they can be compared against the binding results of step (ii); or (c) upscaling or downscaling both the binding results of step (ii) and the MS results of step (iii) so that the results can be compared against one another.

39. The method of claim 38 wherein the upscaling and/or downscaling is carried out so that the maximum binding signal value with respect to either a series of fractions or all fractions analysed is the same as, or corresponds to, the maximum relative abundance with respect to either a series of fractions or all fractions analysed as determined by MS.

40. The method of claim 1, wherein step (iv) comprises the steps of:
a) determining the relative abundance of the polypeptide of interest within each fraction from the mass spectrometry results from step (iii);
b) plotting the binding signal intensity for a polypeptide binding to a specific binding agent detected in step (ii) against each fraction;
c) overlaying the relative abundance data determined in step a) with the binding results of step b); and
d) determining the level of overlap between the mass spectrometry results and the binding results;
or wherein step (iv) comprises the steps of:
a) determining the relative binding signal intensity for a polypeptide binding to a specific binding agent detected in step (ii) within each fraction;
b) plotting the abundance of the polypeptide of interest within each fraction from the mass spectrometry results from step (iii) against each fraction;
c) overlaying the relative binding signal intensity data determined in step a) with the abundance results of step b); and
d) determining the level of overlap between the mass spectrometry results and the binding results.

41. The method of claim 34 wherein a correlation or level of overlap of more than 80%, preferably 85%, more preferably 90%, is indicative of a binding agent that is specific for the polypeptide of interest.

42. The method of claim 34 wherein step (i) forms one or more series of continuous fractions and wherein step (iv) further comprises calculating a wide index, wherein the wide index is calculated by
a) determining the MS centre by determining the fraction with the highest signal intensity or abundance of the polypeptide of interest obtained from the MS data in relation to a series of fractions or in relation to all the fractions;
b) calculating the sum of the binding signal intensity from the binding agent array analysis in step (ii) measured in the fraction corresponding to the MS centre and the two immediate neighbouring fractions on each side of the MS centre divided by the sum of the binding signal intensity measured in either a series of fractions or all fractions.

43. The method of claim 42, wherein a wide index of more than 0.70, preferably 0.80, more preferably 0.90 is indicative of a binding agent that is specific for the polypeptide of interest.

44. The method of claim 34 wherein step (i) forms one or more series of continuous fractions and wherein step (iv) further comprises calculating a core index, wherein the core index is calculated by:
a) determining the MS centre by determining the fraction with the highest signal intensity or abundance of the polypeptide of interest obtained from the MS data in relation to a series of fractions or in relation to all the fractions;
b) calculating the sum of the binding signal intensity from the binding agent array analysis in step (ii) measured in the fraction corresponding to the MS centre and the two immediate neighbouring fractions divided by the sum of the binding signal intensity measured in either a series of fractions or all fractions.

45. The method of claim 44, wherein a core index of more than 0.70, preferably 0.80, more preferably 0.90 is indicative of a binding agent that is specific for the polypeptide of interest.

46. The method of claim 34 wherein step (iv) further comprises calculating a signal index, wherein the signal index is calculated by dividing the maximal binding signal intensity from the binding agent array analysis in step (ii), taken from either a series of fraction or all analysed fractions, by the median binding signal intensity.

47. The method of claim 46 wherein a signal index of more than 3, preferably 4, more preferably 5, is indicative of a binding agent that has an adequate level of sensitivity.

48. The method of claim 34 wherein step (iv) further comprises determining the absolute signal intensity, wherein the absolute signal intensity is the maximal binding signal intensity from the binding agent array analysis measured in step (ii) for a particular binding agent.

49. The method of claim 48 wherein an absolute signal intensity of more than 1500, preferably 2500, more preferably 3500, is indicative of a binding agent that has an adequate level of sensitivity.

50. The method of claim 1 wherein the correlation is either carried out or determined using a computer algorithm.

51. The method of claim 1 wherein in step (iii) the amino acid sequences of the polypeptides is determined.

52. The method of claim 1 wherein the mass spectrometry carried out in step (iii) is liquid chromatography mass spectrometry.

53-56. (canceled)

57. The method of claim 1, further comprising the step of stable isotope metabolic labelling of cells prior to step (i).

58-59. (canceled)
